(19) United States
(12) Patent Application Publication
(10) Pub. No.: US 2024/0252545 A1
(43) Pub. Date: Aug. 1, 2024

(54) Methods for Making Stem Cell-Derived Enteric Neural Crest Cells and Their Use in Enteric Neuropathy Treatment

(71) Applicant: Children's Hospital Los Angeles, Los Angeles, CA (US)

(72) Inventors: Tracy Grikscheit, Los Angeles, CA (US); Laura-Marie Nucho, Los Angeles, CA (US)

(21) Appl. No.: 18/559,119

(22) PCT Filed: May 9, 2022
(86) PCT No.: PCT/US22/28385

§ 371 Date: Nov. 6, 2023

Related U.S. Application Data
(60) Provisional application No. 63/185,961, filed on May 7, 2021.

(51) Int. Cl. A61K 35/30; A61P 25/02; C12N 5/0797

(52) U.S. Cl. CPC A61K 35/30; A61P 25/02; C12N 5/0623; C12N 2500/38; C12N 2501/115; C12N 2501/415; C12N 2506/45

(57) ABSTRACT

Provided herein are compositions and methods to treat enteric neuropathies/or slow gut motility comprising intra-muscularly administering enteric neural crest cells (ENCCs) to a intestine of a subject in need thereof, wherein the ENCCs are differentiated from stem cells in vitro.

### BACKGROUND OF THE INVENTION

Enteric nervous system (ENS) development and function are governed by a broad array of regulatory molecules that are only partially known. Disruption of signaling pathways from congenital or acquired defects results in varying degrees of pathology (Lake and Heuckeroth, 2013). Among congenital enteric neuropathies, Hirschsprung's disease (HD) is the most common and is reported to arise from impaired migration of enteric neural crest cells (ENCCs) within the gut during fetal development, leading to functional obstruction from an inability to relax intestinal smooth muscle (Furness, 2012). Surgical resection of the distal aganglionic segment of intestine in HD decreases mortality but does not result in complete resolution of symptoms (Laughlin et al., 2012: Sulkowski et al., 2014).

Accordingly, there is an important unmet need for effective therapies for treating HD and other enteric neuropathies. The present disclosure meets these needs and offers other related advantages.

### SUMMARY OF THE INVENTION

According to one aspect of the present disclosure, there is provided a method for treating an enteric neuropathy comprising administering enteric neural crest cells (ENCCs) to a muscular wall of the intestine in a subject in need thereof, wherein the ENCCs are differentiated from pluripotent stem cells in vitro. In some embodiments, the stem cells are embryonic stem (ES) cells or induced pluripotent stem cells (iPSCs). In a more particular embodiment, the iPSCs comprise the cell line referred to as LiPSC-GR1.1.

In some embodiments, the enteric neuropathy treated in accordance with the present disclosure is Hirschsprung's Disease (HD). In some embodiments, the enteric neuropathy is a total intestinal aganglionosis form of HD or a long segment form of HD. In still other embodiments, the enteric neuropathy treated according to the present disclosure is selected from aganglionosis of the rectosigmoid colon (short segment disease), aganglionosis of the sigmoid colon (long segment disease), congenital aganglionosis in Hirschsprung disease (HD), autoimmune-mediated loss of neuronal subtypes in esophageal achalasia and Chagas disease, degenerative neuropathies in chronic intestinal pseudo-obstruction or gastroparesis, enteric neuropathy caused by nerve agents (e.g., Gulf War syndrome) and/or diabetic gastroparesis.

The cells can be administered to the subject using any suitable technique. In some embodiments, the cells are administered by injection to a muscular wall of the intestine. In some embodiments, the cells are administered to two or more different locations of the intestine. In more particular embodiments, the cells are administered by bilateral injections about every 1 to 3 centimeters in the muscular wall of the intestine. In other more particular embodiments, the cells are administered about every 2 centimeters in the muscular wall of the intestine.

The number of cells administered to a subject in need will vary depending on the particular situation. In some embodiments, each dose of cells administered comprises about 1×10⁵ to 1×10⁹ cells. In a more particular embodiment, each dose of cells administered comprises about 1×10⁷ cells.

In some embodiments, the cells are cryopreserved and thawed prior to administration to the subject.

In some embodiments, the cells are suspended in a pharmaceutically acceptable carrier prior to administration to the subject.

In some embodiments, the methods of the disclosure further comprise administering one or more additional agents to the subject, such as those selected from the group consisting of a preservative, a cytokine, a pH buffering agent and a migration promoting agent. In a more particular embodiment, the methods of the disclosure further comprise administering one or more migration promoting agent to the subject selected from the group consisting of Glial cell-derived neurotrophic factor (GDNF), Nerve growth factor (NGF), Brain Derived Neurotrophic Factor (BDNF), Pepstatin A or a combination thereof.

In some embodiments, the subject to whom the cells are administered has been clinically immunosuppressed prior to administration of the cells.

In some embodiments, the cells that are administered to the subject are allogeneic to the subject. In some embodiments, the cells are genetically modified cells. In some embodiments, the cells are genetically modified to overexpress and/or under express a gene or gene product of interest. In some embodiments, the cells have been genetically modified to evade immune system surveillance. In some embodiments, the cells are universal cells.

According to another aspect of the present disclosure, there are provided compositions comprising ENCCs differentiated from stem cells in vitro (e.g., from LiPSC-GR1.1 cells) and a cryopreservative,

According to another aspect of the present disclosure, there are provided neurospheres comprising ENCCs differentiated from stem cells in vitro (e.g., from LiPSC-GR1.1 cells). In certain embodiments, the neurospheres comprise a mixture of cells. In some embodiments, the mixture of cells are positive for expression of HNK-1, positive for expression p75, negative for expression Oct4 and negative for expression Nanog, wherein the expression of HNK-1, p75, Oct4 and/or Nanog is from one cell or each marker is expressed individually or in combination on different cells. In some embodiments, a cell is considered negative that it is negative for a certain threshold of expression, meaning that that about 1-100 cells of 10,000 can be positive but such a low level is still considered negative in this context.

In some embodiments, neurospheres are provided that comprise a mixture of cells, wherein the mixture of cells comprises
a cell with at least one marker selected from the group consisting of PRSS23, AL359091.1, ID3, NPPC, HES5 or a combination thereof:
a cell with at least one marker selected from the group consisting of H4C3, PCLAF, RRM2, TYMS, CLSPN, ZWINT, TUBAIB, MAD2LI, PCNA, IER2, GINS2 or a combination thereof:
a cell with at least one marker selected from the group consisting of UNG, MCM3, DUT, MCM5, MCM7, NASP, CCND1 or a combination thereof:
a cell with at least one marker selected from the group consisting of UBE2C, ARL6IP1, TOP2A, CCNB1, CENPF, PTTG1, KPNA2, PLK1, TUBB4B, CDC20 or a combination thereof:
a cell with at least one marker selected from the group consisting of PTMS, BIRC5, CCNB2, HSP90B1, HMGB2 or a combination thereof:
a cell with at least one marker selected from the group consisting of IGFBP2, H3-3B, P4HA1, ETFB, WSB1, LDHA, H1-0, DLK1, BNIP3, HSPB1 or a combination thereof:
a cell with at least one marker selected from the group consisting of MGLL, LINC01198, PLP1, MPZ, SCRG1, IGFBP5, FGFBP3, RARRES2, APOE, RHOB or a combination thereof:
a cell with at least one marker selected from the group consisting of TUBB, MZT1, BUB3, CKS2, CKS1B, LMNB1 or a combination thereof:
a cell with at least one marker selected from the group consisting of DLL3, GADD45G, IGFBPL1, HES6, RGS16, NHLH1, TAGLN3, CRABP1, NEUROG1, JAG1 or a combination thereof:
a cell with at least one marker selected from the group consisting of NEFM, NEFL, STMN2, ONECUT2, DAAM1, MAP1B, TUBA1A, MLLT11 or a combination thereof:
a cell with at least one marker selected from the group consisting of PAX6, SFRP2, NR2F1, CLU, MYO10, MIAT, LHX5-AS1, TPBG, ID4, TCF7L2 or a combination thereof: and/or
a cell with at least one marker selected from the group consisting of TMSB4X, SST, PPP1R17, S100A10, RGS10 or a combination thereof,

wherein the markers are expressed individually on a cell or in combinations on a cell. According to another aspect of the present disclosure, there are provided methods for producing ENCCs and/or neurospheres comprising ENCCs, comprising the steps of:
a) expanding stem cells in suspension:
b) contacting the stem cells of a) with at least one inhibitor of SMAD, at least one activator of the WNT pathway, FGF2 and retinoic acid and culturing said stem cells for a period of time and under conditions sufficient to differentiate said stem cells to enteric neural crest cells (ENCCs) neurospheres: and
c) isolating said enteric neural crest cells (ENCCs) neurosphere of b).

In some embodiments of this aspect of the invention, the stem cells are iPSCs, such as the cell line LiPSC-GR1.1, or embryonic stem cells.

In some embodiments, the inhibitor of SMAD is LDN193189 and/or SB431542.

In some embodiments, the activator of the WNT pathway is CHIR99021.

In still further embodiments of this aspect of the disclosure, the stem cells are
(a) cultured in medium with at least one inhibitor of SMAD and FGF2 for a period of time:
(b) the cells of a) are then cultured in a medium with at least one inhibitor of SMAD, FGF2 and at least one activator of the WNT pathway for a period of time:
(c) the cells of b) are then cultured in a medium with at least one inhibitor of SMAD, FGF2, at least one activator of the WNT pathway and retinoic acid for a period of time:
(d) the cells of c) are then cultured in a medium with at least one inhibitor of SMAD, at least one activator of the WNT pathway and retinoic acid for a period of time: and
(e) the cells of d) are then cultured in a medium with FGF2 and at least one activator of the WNT pathway for a period of time.

In other embodiments, cells or neurospheres produced according to these methods of the disclosure are subsequently cryopreserved.

According to still another aspect of the present disclosure, there are provided ENCCs and neurospheres produced according to the methods described herein.

According to still another aspect of the present disclosure, there are provided pharmaceutical compositions comprising ENCCs and/or neurospheres produced according to the methods described herein in combination with a pharmaceutically acceptable carrier.

According to yet another aspect of the present disclosure, there are provided methods for treating an enteric neuropathy comprising injecting the ENCC and/or neurospheres produced according to the methods described herein to the intestine in a subject in need thereof.

### BRIEF DESCRIPTION OF THE DRAWINGS

FIG. 1. Schematic outlining the ENCC differentiation process.

FIG. 2. IF analysis of pluripotent cells and ENCC derived from LiPSC-GR1.1. Pluripotent LiPSC-GR1.1 cells and ENCC cells were analyzed (CHLA and COH). A-C) H&E staining, D-F) pluripotent marker Oct4 (green) staining, G-I) pluripotent marker Nanog (green) staining, J-L) neural marker Tuj1 (green) M-O) glial marker S100b (red), P-R) intestinal epithelial market Ecad (green), S-U) proliferation marker PCNA (red) staining, and V-X) human marker lamin (red). Nuclei labeled with DAPI (blue).

FIG. 3. Long-term in vitro culture of ENCC derived from LiPSC-GR1.1 in GDNF-containing media results in further differentiation into enteric glia and diverse classes of neurons. A) neural marker Tuj1 (red), B) marker RET (red), C) marker GABA (green), D) glial marker s1000 (red), E) marker EDNRB (red), F) serotongeric neuron marker 5-HT (red), G) marker TrkC (green), H) marker ChAT (green) and neural marker Tuj1 (red), I) Calrentinin (red) and neural marker Tuj1 (green), J) nNos (red) and neural marker Tuj1 (green), K) PHOX2B (red) and neural marker Tuj 1 (green). Nuclei are labeled with DAPI (blue).

FIG. 4. Flow cytometry analysis detecting p75 and HNK-1 on Day 15. For flow cytometry analysis. ENCC neurospheres are dissociated into single cells, and evaluated for A) unstained cells 99.5% double negative, B) single stained enteric neural crest marker p75 (CD271) on the y-axis (98.3%), C) single stained enteric neural crest marker HNK-1 (CD57) on the x-axis (70.9%), and D) dual stained for p75 and HNK1 (64.6%).

FIG. 5. Flow analysis on fresh ENCC on day 15 of differentiation. A) unstained ASCENT 98.9% double negative, B) Isotype control with ENCC cells 99.8% double negative, and C) LiPSC-GR1.1 ENCC dual stained for CD57 and CD271 is 95.7%.

FIG. 6. qPCR data of pluripotent LiPSC-GR1.1 and Day 15 ENCC. A) Oct4 and Nanog pluripotency markers, and B) enteric neural expression markers EDNRB and RET from three unique lots of adherent ENCC, C) qPCR analysis of adherent (dark blue) and suspension culture (light blue) show similar expression levels of key genetic markers in the directed differentiation. The asterisk indicates significance relative to day 0) for the two conditions (+2 SEM,>95% confidence).

FIG. 7. QPCR analysis of day 11 and day 15 fresh ENCC. Day 11 and day 15 cells were normalized to GAPDH and compared to Day 0 (undifferentiated) LiPSC-GR1.1 iPSC cells. A) GAPDH housekeeping gene, B-C) Pluripotency genes Oct4 and Nanog, D) Sox9 intestinal stem cell marker, E-G) neural plate boarder markers Pax3. Pax7, and ZIC1, H-J) neural crest cell specifier genes Sox10, APA2, and SNAIL2, K-N) vagal neural crest cell genes HOXA2, HOXB3, HOXB5, and HOXB7, O) PHOX2B enteric neural crest cell progenitor gene, P-T) Neural enteric neural crest cell genes p75, EDNRB, END3, RET, and GDNF.

FIG. 8. In vitro migration studies. A) Reproducibly sized wounds for all cell lines at 0 hours and after 72 hours of cell migration. Wounds in all cell lines had closed by 72 hours. B) Graph displaying rate of cell migration over time by analyzing the diameter of the wound every 24 hours. C) Wound closure in pixels over 72 hours shows how much of the wound closes every 24 hours for H9, LiPSC-GR1.1, and ESI-017 by measuring the circumference of the wound every 24 hours. The closure and migration rate of ENCC derived from LiPSC-GR1.1 is not significantly different from the embryonic lines.

FIG. 9. ENCC-HIO-TESI from PHOX2B-/- HIO. A-D) images of whole explant at 8 weeks, E-F) brightfield image of pluripotent LiPSC-GR1.1 and day 15 ENCC, G-H) H&E stains of explants and cells, M-R) pluripotency marker Oct4 (green) and human mitochondira marker (red), S-X) neural marker Tuj1 (green) and human marker lamin (red), Y-D′) gial marker s 100b (green and human marker lamin (red), E′-J′) proliferactive cell marker PCNA (green) and human marker lamin (red).

FIG. 10. ENCC-HIO-TESI generated from PHOX2B-/- HIO and LiPSC-GR1.1 ENCC or PHOX2B-/- ENCC (control). A-E) Images of native human post-natal ileum and 12 week explants, F-J) H&E staining of native human post-natal ileum and 12 week explants, K-O) pluripotent marker Oct4 (red) and proliferation marker PCNA (green) staining, P-T) neural marker Tuj1 (green) and human marker lamin (red), U-Y) neural marker Tuj1 (green) and glial marker S100b (red), Z-DD) neural marker Tuj1 (green) and calculim binding protein Calbindin (red), EE-II) neural marker Tuj1 (green) and ChAT-positive excitatory neurons (red), JJ-NN) neural marker Tuj1 (green) and NOS-positive inhibitory neurons (red).

FIG. 11. IF detection of ENCC derived from LiPSC-GR1.1 in swine intestine 4 weeks after injection with immunosuppression. A) Cecum from pig #6267 stained for Tuj1 (green), human lamin (red), and DAPI (blue) 4 weeks post-injection. The arrows are identifying ENCC cells co-stained for Tuj1 and lamin. Nuclei are labeled with DAPI (blue). Scale bar is 50 mm. B) Enlarged image of ENCC cell expressing human lamin, C) Enlarged image of ENCC cell expressing Tuj1.

FIG. 12. Single cell analysis of ENCC derived from either LiPSC-GR1.1 (WT) or disease state cell line (Phox2B mutation) after differentiation protocol.

FIG. 13. ENS components derived from ASCENT form a neural network within the intestinal wall of HIO-TESI, a model of human aganglionic intestine. A) Light-sheet microscopy demonstrates interconnected neural networks (Tuj1) within the muscular intestinal wall (SMA) in 12-week co-implanted ASCENT-HIO-TESI with SMA and Tuj1 detected by immunofluorescence. B) HIO-TESI with or without ASCENT develops intrinsic pacemaker cells similar to native intestine, known as the interstitial cells of Cajal (ICCs, c-KIT+), which are responsible for autonomic rhythmicity and are found in the submucosal and inter/intramuscular layers¹. C-KIT-positive ICCs in ASCENT-HIO-TESI closely associate with neurons in the myenteric plexus, similar to human fetal intestine. In control HIO-TESI without ASCENT, however, C-KIT-positive ICCs are randomly distributed within the myenteric layers.

FIG. 14. A) Light-sheet microscopy of HIO-TESI and ASCENT-HIO-TESI demonstrates the absence of TUJ1-positive neurons (red) within the muscularis propria (SMA, green) without co-implantation of ASCENT. TUJ1-positive neural networks (red) integrate within the smooth muscle in ASCENT-HIO-TESI (SMA, green). Scale bars, 3 mm (n=5). B) Confocal microscopy identified enteroendocrine cells (CHGA, green) within HIO-TESI and ASCENT-HIO-TESI. A TUJ1-positive neuronal axon forms synaptic connections with a CHGA-positive enteroendocrine cell. Scale bars, 10 mm (n=5). 3D rendering identifies the co-stained connection from the epithelium to the ENS formed from ASCENT.

FIG. 15. Cross-sections of ASCENT HIO-TESI and 17-week old human fetal intestine were imaged and the area of positive staining as a percent of the total tissue area per section for Tuj1, SMA, GFAP and s100β was calculated. ASCENT-HIO-TESI demonstrated a similar area of Tuj1 (4.52%±4.67 v. 6.19%±2.45), SMA (16.52%±10.42 v. 15.01%±2.35), s100β (3.68%±1.57 v. 4.33%±4.25) and a lesser number of GFAP (2.30%±1.07 v. 5.31%±3.79) compared to human fetal ileum (p<0.05).

FIG. 16. Ku-80 staining (human marker, brown) identifies neural structures traversing the intestinal wall muscle layers in pig 6577 connecting to the pig's native Meissner's plexus.

FIG. 17. Double positive TUJ1 and LAMIN cells in pig 6267 after injection of six doses of E6 ASCENT, harvested at 4 weeks.

FIG. 18. An example of an ASCENT-HIO-TESI sample after treatment with methylene blue during video analysis with Tracker. The Tracker program is a video analysis and modeling tool built on the Open Source Physics (OSP) Java framework.

FIG. 19. Data points collected by Tracker were exported to and graphed in Microsoft Excel to quantify the amplitude of contractions from 3-month HIO-TESI with and without ASCENT.

FIG. 20. Amplitude distribution for four conditions calculated from XY coordinate and Single Vector Data. The black line represents the median, the box represents the interquartile range, and the whiskers represent the maximum and minimum values of the data set.

FIG. 21. Final ASCENT product captured after differentiation at COH with scale bar=200 um.

FIG. 22. Children with LSA or TA will have multiple biopsies of the intestine prior to dividing the intestine where there are abundant components of the ENS above the division (shown as blue ganglia in this figure) and an absence below the division (shown as minus signs).

FIG. 23. Positioning of the stoma and mucus fistula (MF) on the child's abdomen after leveling biopsies are completed.

FIG. 24. Endoscopic surveillance for evidence of obstruction, mass formation, biopsy, and assessment of motility will be performed through the MF at six months and one year.

FIG. 25. Illustrative staggered dose escalation protocol.

FIG. 26. Illustrative injection administration strategy.

FIG. 27. Illustration of how the intestine is reconnected to natively ganglionated intestine (blue), when ASCENT has adequately repopulated the previously aganglionic segment with new components of the ENS (red).

FIG. 28. Vagal-specific ganglia and a wide array of neuronal subtypes are present in ASCENT-HIO-TESI immunofluorescent staining of human fetal ileum and ASCENT-HIO-TESI. A) TUJ1-positive submucosal and myenteric ganglia express vagal and enteric neural crest cell markers PHOX2B and TRKC/RET/EDNRB: submucosa (SM), circular muscle (CM), longitudinal muscle (LM). Scale bars, 100 mm (n=6). B) Various subclasses of enteric neurons were identified, including excitatory (CHAT/TUJ1), inhibitory (nNOS/TUJ1), and sensory neurons (5-HT/TUJ1). Calbindin- and calretinin-positive neurons were also restored in ASCENT-HIO-TESI; nuclei are labeled with DAPI (blue). Scale bars, 100 mm (n=11).

### DETAILED DESCRIPTION OF THE INVENTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### General Methods

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor

Press: Oligonucleotide Synthesis (P. Herdewijn, ed., 2004); Animal Cell Culture (R. I. Freshney), ed., 1987): Methods in Enzymology (Academic Press, Inc.): Handbook of Experimental Immunology (D. M. Weir & C. C. Blackwell, eds.): Gene Transfer Vectors for Mammalian Cells (J. M. Miller & M. P. Calos, eds., 1987): Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991): Short Protocols in Molecular Biology (Wiley and Sons, 1999): Manual of Clinical Laboratory Immunology (B. Detrick, N. R. Rose, and J. D. Folds eds., 2006); Immunochemical Protocols (J. Pound, ed., 2003): Lab Manual in Biochemistry: Immunology and Biotechnology (A. Nigam and A. Ayyagari, eds. 2007): Immunology Methods Manual: The Comprehensive Sourcebook of Techniques (Ivan Lefkovits, ed., 1996): Using Antibodies: A Laboratory Manual (E. Harlow and D. Lane, eds., 1988); and others.

Before the present methods and compositions are described, it is to be understood that this invention is not limited to a particular method or composition described, as such may, of course, vary. The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions:

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Any methods and materials similar or equivalent to those described herein can be used in the practice or testing of the present invention. Specific and preferred values listed below for radicals, substituents, and ranges are for illustration only: they do not exclude other defined values or other values within defined ranges for the radicals and substituents.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “isolated” refers to a factor(s), cell or cells which are not associated with one or more factors, cells or one or more cellular components that are associated with the factor(s), cell or cells in vivo.

“Cells” include cells from, or the “subject” is, a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, primate (e.g., monkey, ape, gorilla, chimpanzee, or orangutan), rat, sheep, goat, cow and bird.

The term “pluripotent cells” refers to cells that can self-renew and proliferate while remaining in an undifferentiated state and that can, under the proper conditions, be induced to differentiate into any of the three germ layers: endoderm (e.g., the stomach lining, gastrointestinal tract, lungs, etc.). mesoderm (e.g., muscle, bone, blood, urogenital tissue, etc.) or ectoderm (e.g., epidermal tissues and nervous system tissues). The term “pluripotent cells,” as used herein, encompasses embryonic stem cells and other types of stem cells, including fetal, amniotic, or somatic stem cells. The term “pluripotent stem cells.” as used herein, also encompasses “induced pluripotent stem cells”, “iPSs” or “iPSCs” (e.g., such as LiPSC-GR1.1), a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of reprogramming proteins or by the exogenous application of certain proteins. Methods of generating and characterizing iPS cells are well known in the art and include those described herein and found in Application Nos. US20090047263, US20090068742, US20090191159, US20090227032, US20090246875, and US20090304646, and Zhou et al., Stem Cells 27 (11): 2667-74 (2009): Huangfu et al., Nature Biotechnol. 26 (7): 795 (2008): Woltjen et al., Nature 458 (7239): 766-770 (2009): Zhou et al.,Cell Stem Cell 8:381-384 (2009) and Omole and Fakoya, PeerJ. 2018:6:e4370 (2018), Table 2: each of which is incorporated by reference in its entirety.

As used herein. “reprogramming factors” refers to one or more, e.g., a cocktail, of biologically active factors that act on a cell to alter transcription, thereby reprogramming a cell to a different differentiation state. For example, the reprogramming factors may reprogram the cell to a state of pluripotency. Alternatively, the reprogramming factors may reprogram the cell to adopt the characteristics of a different type of somatic cell. In methods where reprogramming factors are provided to cells, i.e., the cells are contacted with reprogramming factors, these reprogramming factors may be provided to the cells individually or as a single composition, that is, as a premixed composition, of reprogramming factors. The factors may be provided at the same molar ratio or at different molar ratios. The factors may be provided once or multiple times in the course of culturing the cells of the subject invention.

The phrase “stem cells” includes, but is not limited to, embryonic stem cells (including human embryonic stem cells (hESC)), somatic stem cells (e.g., human), induced pluripotent stem cells (iPSC (e.g., human: such as LiPSC-GR1.1)), and the like. The stem cells can be allogeneic, xenogeneic or autogenic.

“Progenitor cells” are cells produced during differentiation of a stem cell that have some, but not all, of the characteristics of their terminally differentiated progeny. Defined progenitor cells may be committed to a lineage, but not to a specific or terminally differentiated cell type.

“Self-renewal” refers to the ability to produce replicate daughter stem cells having differentiation potential that is identical to those from which they arose. A similar term used in this context is “proliferation.”

“Expansion” refers to the propagation of a cell or cells without differentiation.

“Engraft” or “engraftment” refers to the process of cellular contact and incorporation into an existing tissue of interest in vivo.

“Cytokines” refer to cellular factors that induce or enhance cellular movement, such as homing of stem cells, progenitor cells or differentiated cells. Cytokines may also stimulate such cells to divide.

“Differentiation factors” refer to cellular factors, preferably growth factors or factors that induce lineage commitment.

As used herein, “treat,” “treating” or “treatment” includes treating, preventing, ameliorating, or inhibiting an injury or disease related condition and/or a symptom of an injury or disease related condition.

An “effective amount” generally means an amount which provides the desired local or systemic effect. For example, an effective dose is an amount sufficient to affect a beneficial or desired clinical result. Said dose could be administered in one or more administrations and could include any preselected number of cells. The precise determination of what would be considered an effective dose may be based on factors individual to each subject, including their size, age, injury and/or disease or being treated and amount of time since the injury occurred or the disease began. One skilled in the art, specifically a physician, would be able to determine the number of cells that would constitute an effective dose.

“Co-administer” can include simultaneous and/or sequential administration of two or more agents.

As described herein, “enteric neuropathy” refers to a degenerative neuromuscular condition of the digestive system. In essence the gut stops functioning, due to degradation of the nerves and/or muscles. The condition generally affects all parts of the digestive tract. “Enteric neuropathy” is also called “intestinal pseudo-obstruction.”

Unless specified, “enteric neural crest cells”, also referred to as “ENCCs” refer to ENCCs derived from stem cells in vitro, such as from iPSCs.

As used herein, “ASCENT” refers to stem cell-derived ENCCs, such as those produced according to the present disclosure, for use in the treatment of enteric neuropathies.

The terms “comprises,” “comprising,” and the like can have the meaning ascribed to them in U.S. Patent Law and can mean “includes,” “including” and the like. As used herein, “including” or “includes” or the like means including, without limitation.

As used herein, the terms “identical” or percent “identity” in the context of two or more nucleotide sequences or amino acid sequences, refers to having the same or having a specified percentage of amino acid residues or nucleotides that are the same, when compared and aligned for maximum correspondence, as measured using one of the sequence comparison algorithms described herein, e.g., the Smith-Waterman algorithm, or by visual inspection.

As used herein, the term “sequence identity” refers to the degree of identity between nucleotides or amino acids in two or more aligned sequences, when aligned using a sequence alignment program. The term “% homology” is used interchangeably herein with the term “% identity” herein and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Sequence identity may be determined by aligning sequences using any of a number of publicly available alignment algorithm tools, e.g., the local homology algorithm of Smith & Waterman, Adv. Appl. Math. 2:482 (1981), the global homology alignment algorithm of Needleman & Wunsch, J Mol. Biol. 48: 443 (1970), the search for similarity method of Pearson & Lipman, Proc. Nat'l. Acad. Sci. USA 85: 2444 (1988), computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J Mol. Biol. 215: 403-410 (1990), with software that is publicly available through the National Center for Biotechnology Information (www.ncbi.nlm.nih.gov/), or by visual inspection (see generally, Ausubel et al., infra). In some embodiments, any time the term sequence identity is used herein, it refers to such identity as determined using global alignment.

As used herein, the terms “native”, or “wild type” when used in the context of a polynucleotide or polypeptide herein, refers to a polynucleotide or polypeptide sequence that is found in nature: i.e., that is present in the genome of a wild type virus or cell.

As used herein, the terms “variant”, when used in the context of a polynucleotide or polypeptide herein (such as a variant growth factor or the like used in the methods of the present disclosure), refers to a mutant of a native polynucleotide or polypeptide having less than 100% sequence identity with the native sequence or any other native sequence. Such variants may comprise one or more substitutions, deletions, or insertions in the corresponding native gene or gene product sequence. In some cases, a variant will have at least about 70%, 75%, 80%, 85%, 90%, 95% or 99% identity with its native sequence. The term “variant” also includes fragments of the native gene or gene product, and mutants thereof, e.g., fragments comprising one or more substitutions, deletions, or insertions in the corresponding native gene or gene product fragment. In some embodiments, the variant retains a functional activity of the native gene product, e.g., ligand binding, receptor binding, protein signaling, etc., as known in the art.

As used herein, the term “fragment,” when referring to a recombinant protein or polypeptide (e.g., to a growth factor such as FGF2) of the invention, refers to a polypeptide having an amino acid sequence which is the same as part of, but not all of, the amino acid sequence of the corresponding full-length protein or polypeptide, which retains at least one of the functions or activities of the corresponding full-length protein or polypeptide. The fragment preferably includes at least 20, 30, 40, 50, 60, 70, 80, 90, 100, 150, 200, 250, 300, 350, 400, 450 or 500 or more contiguous amino acid residues of the full-length protein or polypeptide. In some embodiments, the fragment preferably includes at least 50%, 60%, 70%, 80% or 90% or more contiguous amino acid residues of the full-length protein or polypeptide.

As used herein, the terms “biological activity” and “biologically active” refer to the activity attributed to a particular gene product, e.g., RNA or protein, in a cell line in culture or in vivo. For example, the “biological activity” of an RNAi molecule refers to the ability of the molecule to inhibit the production of a polypeptide from a target polynucleotide sequence.

As used herein, the terms “native” or “wild type” when used in the context of a cell herein, refer to a cell that comprises a genome found in nature, i.e., a genome that has not been engineered to comprise a modification. Thus, for example, a somatic cell that has been harvested from an individual would be a wild type cell. A pluripotent stem cell that has been reprogrammed from that somatic cell would also be a wild type cell. In contrast, a somatic cell or a pluripotent stem cell that has been genetically modified would be a “non-naturally occurring” cell.

As used herein, the term “introducing” refers to contacting a cell, tissue, or subject with a vector for the purposes of delivering a DNA, RNA, or protein to the cell or cells. Such administering or introducing may take place in vivo, in vitro or ex vivo. A vector for expression of a gene product may be introduced into a cell by transfection, which typically means insertion of heterologous DNA, RNA or protein into a cell by physical means (e.g., calcium phosphate transfection, electroporation, microinjection or lipofection), or transduction, which typically refers to introduction by way of a virus or a bacteriophage.

As used herein, the terms “transformation,” “transfection,” “transduction,” or “infection” refer to the delivery of a heterologous DNA, RNA or protein to the interior of a cell, e.g., a mammalian cell, an insect cell, a bacterial cell, etc. by a vector. A vector used to “transform,” “transfect,” “transduce,” or “infect” a cell may be a plasmid, minicircle DNA, synthetic RNA, RNP, lipid nanoparticle, extracellular vesicle, exosome, or other vehicles. Typically, a cell is referred to as “transduced”, “infected,” “transfected” or “transformed” dependent on the means used for administration, introduction or insertion of heterologous DNA, RNA, or protein (i.e., the vector) into the cell. The terms “transfected” and “transformed” are used interchangeably herein to refer to the introduction of heterologous DNA, RNA or protein by non-viral methods, e.g., electroporation, calcium chloride transfection, lipofection, etc. The terms “transduced” and “infected” are used interchangeably herein to refer to introduction of the heterologous DNA or RNA to the cell in the context of a viral particle.

The term “host cell”, as used herein refers to a cell which will be or has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original, transduced, infected, transfected or transformed cell and progeny thereof.

As used herein, a “therapeutic” composition refers to a composition that, when administered, confers a beneficial effect on a subject. Thus, for example, a therapeutic cell composition refers to a cell composition that, when grafted into an individual, confers a beneficial effect on the individual in which it is present, or on a mammal in which the cell composition is grafted. Similarly, for example, a therapeutic gene refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic.

The terms “treatment”, “treating” and the like are used herein to generally refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal and includes: (a) preventing the disease from occurring in a subject which may be predisposed to the disease but has not yet been diagnosed as having it: (b) inhibiting the disease, i.e., slowing or arresting its development: or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

As used herein, “genetic modification” refers to a site of genomic DNA that has been genetically edited or manipulated using any molecular biological method, e.g., methods described herein, e.g., by delivering to a site of genomic DNA an endonuclease and at least one guide RNA (gRNA). Examples of genetic modifications include insertions, deletions, mutations, duplications, inversions, and translocations, and combinations thereof. In some embodiments, a genetic modification is a deletion. In some embodiments, a genetic modification is an insertion. In other embodiments, a genetic modification is an insertion-deletion mutation (or indel), such that the reading frame of the target gene is shifted leading to an altered gene product or no gene product.

As used herein, the term “deletion”, which may be used interchangeably with the terms “genetic deletion” or “knock-out” or “KO”, generally refers to a genetic modification wherein a site or region of genomic DNA is removed by any molecular biology method, e.g., methods described herein, e.g., by delivering to a site of genomic DNA an endonuclease and at least one gRNA. Any number of nucleotides can be deleted. In some embodiments, a deletion involves the removal of at least one, at least two, at least three, at least four, at least five, at least ten, at least fifteen, at least twenty, or at least 25 nucleotides. In some embodiments, a deletion involves the removal of 10-50, 25-75, 50-100, 50-200, or more than 100 nucleotides. In some embodiments, a deletion involves the removal of an entire target gene. In some embodiments, a deletion involves the removal of part of a target gene, e.g., all or part of a promoter and/or coding sequence of a target gene. In some embodiments, a deletion involves the removal of a transcriptional regulator, e.g., a promoter region, of a target gene. In some embodiments, a deletion involves the removal of all or part of a coding region such that the product normally expressed by the coding region is no longer expressed. is expressed as a truncated form, or expressed at a reduced level. In some embodiments, a deletion leads to a decrease in expression of a gene relative to an unmodified cell.

As used herein, the terms “insertion” or “integration”, when used in the context of genomic modification and which may be used interchangeably with the terms “genetic insertion” or “knock-in” or “KI”, generally refers to a genetic modification wherein a polynucleotide is introduced or added into a site or region of genomic DNA by any molecular biological method, e.g., methods described herein, e.g., by delivering to a site of genomic DNA an endonuclease and at least one gRNA. In some embodiments, an insertion may occur within or near a site of genomic DNA that has been the site of a prior genetic modification, e.g., a deletion or insertion-deletion mutation. In some embodiments, an insertion occurs at a site of genomic DNA that partially overlaps, completely overlaps, or is contained within a site of a prior genetic modification, e.g., a deletion or insertion-deletion mutation. In some embodiments, an insertion occurs at a safe harbor locus. An insertion may add a genetic function to a host cell, for example, an increase in levels of an RNA or protein. As will be appreciated by those in the art, this can be accomplished in several ways, including adding one or more additional copies of the gene to the host cell or altering a regulatory component of the endogenous gene to increase expression of the protein that is made.

As used herein, the term “endonuclease” generally refers to an enzyme that cleaves phosphodiester bonds within a polynucleotide. In some embodiments, an endonuclease specifically cleaves phosphodiester bonds within a DNA polynucleotide. In some embodiments, an endonuclease is a zinc finger nuclease (ZFN), transcription activator like effector nuclease (TALEN), homing endonuclease (HE), meganuclease, MegaTAL, or a CRISPR-associated endonuclease. In some embodiments, an endonuclease is an RNA-guided endonuclease. In certain embodiments, the RNA-guided endonuclease is a CRISPR nuclease, e.g., a Type II CRISPR Cas9 endonuclease or a Type V CRISPR Cpfl endonuclease. In some embodiments, an endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or combinations thereof. In some embodiments, an endonuclease may introduce one or more single-stranded breaks (SSBs) and/or one or more double-stranded breaks (DSBs).

As used herein, the term “vector” refers to a composition capable of transporting a nucleic acid, i.e., DNA or RNA, or protein into a cell. One type of vector is a “plasmid”, which refers to a circular double-stranded DNA loop into which additional nucleic acid segments can be ligated. Another type of vector is a bacterial artificial chromosome, or “BAC”. Another type of vector is a viral vector, wherein nucleic acid segments can be ligated into the viral genome. Another type of vector is a non-viral vector, e.g., a lipid nanoparticle or an exosome. Another type of vector is a synthetic RNA. Certain vectors are capable of autonomous replication in a host cell into which they are introduced (e.g., bacterial vectors having a bacterial origin of replication and episomal mammalian vectors). Other vectors (e.g., non-episomal mammalian vectors, e.g., lentivirus) are integrated into the genome of a host cell upon introduction into the host cell, and thereby are replicated along with the host genome. Other vectors, while not capable of autonomous replication, are capable of being maintained extrachromosomally in a host cell in which they are introduced (e.g., minicircles, the genome of AAV vectors). Other vectors (e.g., non-episomal mammalian vectors, the genome of lentivirus vectors) are integrated into the genome of a host cell upon introduction into the host cell, and thereby are replicated along with the host genome. Vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia).

As used herein, the term “heterologous” refers to a composition that is non-native to the rest of the entity to which it is being compared. For example, a polynucleotide introduced by genetic engineering techniques into a plasmid or vector derived from a different species, e.g., a viral genome, is a heterologous polynucleotide. As another example, a promoter operatively linked to a coding sequence with which it is not naturally found linked is a heterologous promoter. As a third example, a gene product, e.g., RNA, protein, not normally encoded by a cell in which it is being expressed is a heterologous gene product. As a fourth example, an expression cassette that is not naturally found in a cell is a heterologous expression cassette.

As used herein, the term “expression cassette” refers to a combination of control elements, e.g., promoter, enhancer(s), Kozak consensus sequence, etc. and a gene or genes to which they are operably linked for expression. An “expression vector” refers to a vector, e.g., plasmid, minicircle DNA, bacterial chromosome (BAC), RNA, virus, and the like, that delivers an expression cassette into a cell.

As used herein, the term “expression” refers to the transcription and/or translation of a coding sequence, e.g., an endogenous gene, a heterologous gene, in a cell.

As used herein, the terms “gene” or “coding sequence” refer to a polynucleotide sequence that encodes a gene product and encompasses both naturally occurring polynucleotide sequences and cDNA. A gene may or may not include regions preceding and following the coding region, e.g., 5′ untranslated (5′ UTR) or “leader” sequences and 3′ UTR or “trailer” sequences, or intervening sequences (introns) between individual coding segments (exons).

As used herein, the term “gene product” refers to the expression product of a polynucleotide sequence such as a polypeptide, peptide, protein or RNA including, for example, a messenger RNA (mRNA), a ribozyme, short interfering RNA (siRNA), microRNA (miRNA), small hairpin RNA (shRNA), guide RNA (gRNA), or circular RNA (circRNA). The terms “polypeptide.” “peptide.” and “protein” are used interchangeably herein to refer to polymers of amino acids of any length. The terms also encompass an amino acid polymer that has been modified: for example, disulfide bond formation, glycosylation, lipidation, phosphory lation, or conjugation with a labeling component.

As used herein, the terms “operatively linked”, “operably linked”, or “in operable linkage” refers to a juxtaposition of genetic elements on a single polynucleotide, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the term “promoter” refers to a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species-specific. Promoters may be “constitutive,” meaning continually active, or “inducible,” meaning the promoter can be activated or deactivated by the presence or absence of biotic or abiotic factors.

As used herein, the term “enhancer” refers to a cis-acting regulatory element that stimulates, i.e., promotes or enhances, transcription of an adjacent genes. By a “silencer” it is meant a cis-acting regulatory element that inhibits, i.e., reduces or suppresses, transcription of an adjacent gene, e.g., by actively interfering with general transcription factor assembly or by inhibiting other regulatory elements, e.g., enhancers, associated with the gene. Enhancers can function (i.e., can be associated with a coding sequence) in either orientation, over distances of up to several kilobase pairs (kb) from the coding sequence and from a position downstream of a transcribed region. Enhancer sequences influence promoter-dependent gene expression and may be located in the 5′ or 3′ regions of the native gene. Enhancer sequences may or may not be contiguous with the promoter sequence. Likewise, enhancer sequences may or may not be immediately adjacent to the gene sequence. For example, an enhancer sequence may be several thousand base pairs from the promoter and/or gene sequence.

The terms “individual,” “subject,” “host,” and “patient,” are used interchangeably herein to refer to any mammalian subject for whom diagnosis, treatment, or therapy is desired, particularly humans.

### DETAILED DESCRIPTION OF THE INVENTION

The present invention relates generally to enteric neural crest cells (ENCCs) derived from stem cells and their use the treatment of enteric neuropathies. As described herein, stem-cell derived ENCCs, e.g., iPSC-derived ENCCs, when administered to a subject in need thereof, can advantageously migrate within the intestine, establish motor or sensory diverse neuronal populations and neuron functions, and effectively improve intestinal motility and/or intestinal epithelial cell health.

### Stem Cell-Derived ENCCs

Therefore, according to one aspect of the present disclosure, there are provided methods for making stem cell-derived ENCCs. The methods generally culturing the stem cells under conditions effective for promoting the differentiation of the stem cells into ENCCs. The stem cells used in such methods can be essentially any suitable stem cell known and available in the art that is capable of being differentiated into ENCCs as describe herein. In some embodiments, the stem cells are human stem cells, and can include, but are not limited to, human embryonic stem cells, somatic stem cells, universal cell lines, and iPSCs.

Embryonic stem cells generally refer to pluripotent stem cells derived from the inner cell mass of a blastocyst, an early-stage preimplantation embryo. Human embryos reach the blastocyst stage 4-5 days post fertilization, at which time they consist of 50-150 cells.

Somatic stem cells generally refer to adult stem cells which are undifferentiated cells, found throughout the body after development that multiply by cell division to replenish dying cells and regenerate damaged tissues.

Induced pluripotent stem cells (also known as iPS cells or iPSCs) generally refer to a type of pluripotent stem cell that can be generated directly from adult cells using any of a variety of known techniques. In some embodiments, iPSCs are typically derived by introducing products of specific sets of pluripotency-associated genes, or “reprogramming factors”, into a given cell type. The original set of reprogramming factors identified as effective for generating iPSCs are the transcription factors Oct4 (Pou5f1), Sox2, cMyc, and Klf4. While this combination is most conventional in producing iPSCs, each of the factors can be functionally replaced by related transcription factors, miRNAs, small molecules, or even non-related genes such as lineage specifiers (Nanog, LIN28, Glis1).

In some embodiments, the iPSC cells used according to the present disclosure comprise the line LiPSC-GR1.1

Isolation, growth and preparation of stem cells can be achieved by methods known and available in the art.

Once a suitable stem cell has been selected, the stem cells are cultured under conditions effective for causing their differentiation into ENCCs. In some embodiments, the stem cells are cultured at the same or different times in the presence of a combination of a SMAD inhibitor, FGF2 or a variant thereof, a Wnt activator and/or retinoic acid.

In some embodiments, the stem cells are cultured in the presence of at least one SMAD inhibitor. In more particular embodiments, the SMAD inhibitor is selected from the group consisting of LDN193189, SB431542, IN 1130, A01, Galunisertib (LY2157299), LY2109761. SB525334, SB505124, GW788388 and LY364947, or a combination thereof, used at a suitable concentration

In some embodiments, the SMAD inhibitor is used at a concentration of between about 10 nM-100 uM, or about 1 uM-50 uM, or about 5 uM-20 uM, or about 10 uM, but may also be used at concentration or range of up to about two orders of magnitude down or two-three orders of magnitude up of a concentration or range described here.

In some embodiments, the stem cells are cultured in the presence of FGF2 or a variant or mimic thereof, at a suitable concentration. In more particular embodiments, FGF2 is used at a concentration of between about 0.05 uM-2 mM, or about 1 uM-1.5 mM, or about 0.58 mM, or at a concentration or range of up to about two orders of magnitude down or two-three orders of magnitude up of a concentration or range described above.

In some embodiments, the stem cells are cultured in the presence of at least one activator of WNT. In more particular embodiments, the WNT activator is selected from the group consisting of CHIR99021, IM-12, AZD2858, CP21R7 (CP21), Isoxazole 9 (ISX-9), Wnt agonist 1, a Wnt protein, Wnt surrogates, LiCl, Ant1.4Br/Ant 1.4Cl, SB-216763, BIO(6-bromoindirubin-3′-oxime), SM04554 (Dalosirvat) and/or LY2090314. In more particular embodiments, the activators of WNT can be used in a concentration of about 1 uM-10 uM, or about 2 uM-8 uM, or about 2 uM-5 uM, or about 3 uM, or at a concentration or range of about two orders of magnitude down or two to three orders of magnitude up of a concentration or range described here.

In some embodiments, the stem cells are cultured in the presence of retinoic acid. In more particular embodiments, the retinoic acid is used in a concentration of about 1 uM-20 uM, or about 5 uM-15 uM, or about 8 uM-12 uM, or about 10 uM, or at a concentration or range of up to two orders of magnitude down or two-three orders of magnitude up of a concentration or range described here.

In some embodiments, the method for producing ENCCs comprises expanding stem cells in suspension. In related embodiments, the stem cells are expanded in suspension in the presence of some or all of the above-described factors for a period of time sufficient to differentiated the stem cells to ENCCs.

In some embodiments, the stem cells are a) cultured in medium with at least one inhibitor of SMAD and FGF2 for a period of time: b) the cells of a) are then cultured in a medium with at least one inhibitor of SMAD, FGF2 and at least one activator of the WNT pathway for a period of time: c) the cells of b) are then cultured in a medium with at least one inhibitor of SMAD, FGF2, at least one activator of the WNT pathway and retinoic acid for a period of time: d) the cells of c) are then cultured in a medium with at least one inhibitor of SMAD, at least one activator of the WNT pathway and retinoic acid for a period of time: and

e) the cells of d) are then cultured in a medium with FGF2 and at least one activator of the WNT pathway for a period of time. In some embodiments, the cells in a) through e) and cultured in neural differentiation media and supplements for a period of time, including but not limited to Neuralbasal media, N2 supplement, and B27 supplement.

In some embodiments, the cells remain in culture for the duration of the differentiation process, without the need for cell sorting.

In some embodiments, the cells are cultured in suspension for the duration of the differentiation process.

In other embodiments, the process is adapted for large scale manufacturing, preferably in suspension.

In some embodiments, the ENCCs produced according to the disclosure are in the form of cellular aggregates referred to as neurospheres.

In some embodiments, the ENCCs produced according to the disclosure are a substantially homogenous population of cells. In other embodiments, the ENCCs are a heterogenous mixture of unique cell populations as described herein.

In some embodiments, after differentiation, the cells are cryopreserved. In more particular embodiments, the cryopreservation is carried out in cryopreservation medias with or without about 10uM Y-27632. In some embodiments, the cryopreservation media may contain additional cryoprotectants such as serum albumin and/or sugars such as Trehalose.

In some embodiments, the stem cells used for producing the ENCCs are stem cells that have been genetically engineered in a desired fashion using techniques well known and available to the person of ordinary skill in the art.

For example, in some embodiments, the stem cells are engineered to overexpress a gene or protein of interest so as to produce a desired or beneficial effect or outcome. In some embodiments, the stem cells are engineered to create a deficiency in a gene or protein of interest so as to produce a desired or beneficial effect or outcome.

In more particular embodiments, the gene that is modified to be over or under expressed in the stem cells is a gene associated with one or more of the following: cell proliferation, cell cycle regulation, cell migration, cell adhesion and/or cell differentiation into neurons/glia/enteroendocrine cells.

In other more particular embodiments, the gene that is modified to be over or under expressed in the stem cells is a GDNF, an integrin, FAK, ITGB4, RET and/or EDNRB.

In still other embodiments, the gene that is modified to be over or under expressed in the stem cells is selected from those listed below.

Functional Genes of ENS

| | | | | | | | |
|---|---|---|---|---|---|---|---|
| Calca | Calcb | Calcrl | Ramp1 | Ramp2 | Ramp3 | Ccl | Cckar |
| Cckbr | Dbh | Adra1a | Adra2a | Adrb1 | Adrb3 | Gal | Gcgr |
| Glp1r | Glp2r | Il7 | Il4ra | Il13ra1 | Nmu | Nmur1 | Nmur2 |
| Nog | Piezo1 | Piezo2 | Sst | Sstr1 | Sstr2 | Sct | Sctr |
| SOX10 | FoxD3 | Shh | PAX3 | B2 subset | RET | GDNF | Phox2B |
| P75 | HAND2 | Phox2a | MASH1 | EDNRB | TrkC | EDN3 | GFRalpha1 |
| NRG1 | ERBB2 | ERBB3 | POU3F2 | | | | |

Cytokines, Chemokines, and their Receptors

| | | | | |
|---|---|---|---|---|
| Cxcl12 | Il10rb | Il11ra1 | Il17rd | Il4ra |
| Il6st | Il7 | Tgfbr3 | Tnfrsf21 | |

### Enteric Neuropathies

The ENCCs described herein, with or without further processing, culturing and/or other desired manipulations, can be used for treating human enteric neuropathies. Normal gastrointestinal (GI) function relies on the enteric nervous system (ENS), a complex network of neurons and glia in the gut wall that regulate motor, sensory, absorptive, secretory, and multiple other aspects of GI homeostasis. Comprised of cells that arise from the embryonic neural crest, the ENS is organized in two major ganglionated plexuses, myenteric and submucosal, and functions independently of central nervous system input. Many diseases of the GI tract have a neuropathic cause and lead to significant morbidity due to abnormalities in motor and/or sensory GI function. Some conditions are developmental in origin, caused by abnormal formation of the ENS. Others are acquired in later life due to infection, immune-mediated inflammation, or neuronal degeneration.

Enteric neuropathies include, but are not limited to, congenital aganglionosis in Hirschsprung disease (HD), autoimmune-mediated loss of neuronal subtypes in esophageal achalasia and Chagas disease, and degenerative neuropathies in some cases of chronic intestinal pseudo-obstruction and gastroparesis.

Hirschsprung Disease (HD) is a congenital neuropathy that presents in childhood and is caused by the failure of the complete migration of the enteric nervous system (ENS) into the distal intestine during in utero development, resulting in aganglionic and nonfunctional intestine of varying lengths. HD occurs from distal toward proximal intestine, meaning that if the ENS fails to migrate past the small intestine, the entire large intestine will lack an ENS and thus lack coordinated propulsion. Prevalence of this disease is approximately 1 in 5000 neonates in the United States (3) and international rates are similar (4). Diagnosis typically occurs early after birth, particularly if longer intestinal segments are involved because the obstruction is obvious, although some diagnoses in patients with shorter involved segments are delayed. Patients present with intestinal obstruction and sometimes life-threatening intestinal infection, termed Hirschsprung Associated Enterocolitis (HAEC). In approximately 80% of patients, aganglionosis is limited to the rectosigmoid colon, also known as short segment disease. 15-20% of those affected demonstrate aganglionosis of the sigmoid colon, known as long segment disease, and in about 5-10% of patients, the entire intestine is affected (5).

Other enteric neuropathies include, but are not limited to, soldiers with battlefield exposure and other gut motility issues. One embodiment provides for the treatment of enteric neuropathy caused by nerve agents (e.g., Gulf War syndrome) (Hernandez et al. (2019) FASEB J. 33(5):6168-6184); Zhou et al. Clin J Pain. 2018 Oct: 34(10): 944-949). Gulf War syndrome or Gulf War illness is a chronic and multi-symptomatic disorder affecting returning military veterans of the 1990-1991 Persian Gulf War (“Gulf War Veterans' Illnesses: Illnesses Associated with Gulf War Service”. United States Department of Veterans Affairs. nd. Archived from the original on 12 February 2012. Retrieved 9 May 2012: Iversen A, et al. 2007). “Gulf War Illness: lessons from medically unexplained symptoms”. Clin Psychol Rev. 27 (7): 842-854; Gronseth GS (May 2005). “Gulf war syndrome: a toxic exposure? A systematic review”. Neurol Clin. 23 (2): 523-540.) A wide range of acute and chronic symptoms have been linked to it, including fatigue, muscle pain, cognitive problems, insomnia, (Gulf War Veterans' Medically Unexplained Illnesses“. Veterans Health. Public Health. U.S. Department of Veterans Affairs) rashes and diarrhea (Gulf War Syndrome”. University of Virginia. Archived from the original on 14 July 2004.). Approximately 250,000 (Stencel, C (9) April 2010). “Gulf War service linked to post-traumatic stress disorder, multisymptom illness, other health problems, but causes are unclear”. National Academy of Sciences. Archived from the original on 14 March 2012. Retrieved 9 May 2012) of the 697,000 U.S. veterans who served in the 1991 Gulf War are afflicted with enduring chronic multi-symptom illness, a condition with serious consequences (Research Advisory Committee on Gulf War Veterans' Illnesses (1 November 2008). “Gulf War Illness and the Health of Gulf War Veterans: Scientific Findings and Recommendations” (PDF). U.S. Department of Veterans Affairs. Archived (PDF) from the original on 9 November 2013. Retrieved 9 May 2012).

### Pharmaceutical Compositions

In some embodiments, ENCC cells described herein may be incorporated into a pharmaceutical composition for administration into a patient, such as a human patient suffering from an enteric neuropathy.

Pharmaceutical compositions can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013): incorporated herein by reference), and in a desired form, e.g., in the form of aqueous solutions.

The cells described herein can be administered in any physiologically compatible carrier, such as a buffered saline solution. Pharmaceutically acceptable carriers and diluents include saline, aqueous buffer solutions, solvents and/or dispersion media. The use of such carriers and diluents is well known in the art. Other examples include liquid media, for example, Dulbeccos modified eagle's medium (DMEM), sterile saline, sterile phosphate buffered saline, Leibovitz's medium (L15, Invitrogen, Carlsbad, Calif.), dextrose in sterile water, and any other physiologically acceptable liquid. Dispersions may also be prepared in glycerol, liquid polyethylene glycols, and mixtures thereof and in oils. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. In many cases, it will be preferable to include isotonic agents, for example, sugars or sodium chloride. The solution is preferably sterile and fluid to the extent that easy syringe ability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosol, and the like. Solutions of the invention can be prepared by using a pharmaceutically acceptable carrier or diluent and, as required, other ingredients enumerated above, followed by filtered sterilization, and then incorporating the hypoimmunogenic cells as described herein.

A solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, intraperitoneal, intraventricular, subretinal, and intravitreal administration. In this connection, sterile aqueous media that can be employed will be known to those of skill in the art in light of the present disclosure. The person responsible for administration will, in any event, determine the appropriate dose for the individual subject. Moreover, for human administration, preparations may meet sterility, pyrogenicity, general safety, and purity standards as required by FDA Office of Biologics standards.

Pharmaceutical compositions comprising cells in a semi-solid or solid carrier are typically formulated for surgical implantation at the site of transplantation or at the affected site of a disease or condition in the subject. It will be appreciated that liquid compositions also may be administered by surgical procedures. In particular embodiments, semi-solid or solid pharmaceutical compositions may comprise semi-permeable gels, matrices, cellular scaffolds and the like, which may be non-biodegradable or biodegradable. For example, in certain embodiments, it may be desirable or appropriate to sequester the hypoimmunogenic cells from their surroundings yet enable the cells to secrete and deliver biological molecules (e.g., a therapeutic agent) to surrounding cells.

In other embodiments, different varieties of degradable gels and networks are utilized for the pharmaceutical compositions of the invention. For example, degradable materials include biocompatible polymers, such as poly(lactic acid), poly(lactic acid-co-glycolic acid), methylcellulose, hyaluronic acid, collagen, and the like.

In another embodiment. one or more hydrogels are used for the pharmaceutical compositions. The one or more hydrogels may include collagen, atelocollagen, fibrin constructs, hydrophilic vinyl and acrylic polymers, polysaccharides such as calcium alginate, and poly (ethylene oxide). Further, the hydrogel may be formed of poly(2-hydroxyethyl methacry late), poly(acrylic acid), self-assembling peptides (e.g., RAD16), poly(methacrylic acid), poly(N-vinyl-2-pyrrolidinone), polyvinyl alcohol) and their copolymers with each other and with hydrophobic monomers such as methyl methacrylate, vinyl acetate, and the like. Also preferred are hydrophilic polyurethanes containing large poly(ethylene oxide) blocks. Other preferred materials include hydrogels comprising interpenetrating networks of polymers, which may be formed by addition or by condensation polymerization, the components of which may comprise hydrophilic and hydrophobic monomers such as those just enumerated. In situ-forming degradable networks are also suitable for use in the invention (see, e.g., Anseth, K S et al. J. Controlled Release. 2002: 78: 199-209; Wang, D. et al., Biomaterials. 2003: 24:3969-3980; U.S. Patent Publication 2002/0022676). These in situ forming materials are formulated as fluids suitable for injection; then may be induced to form a hydrogel by a variety of means such as change in temperature, pH, and exposure to light in situ or in vivo. In one embodiment. the construct contains fibrin glue containing gels. In another embodiment, the construct contains atelocollagen containing gels.

A polymer used to form a matrix may be in the form of a hydrogel. In general, hydrogels are cross-linked polymeric materials that can absorb more than 20% of their weight in water while maintaining a distinct three-dimensional structure. This definition includes dry cross-linked polymers that will swell in aqueous environments, as well as water-swollen materials. A host of hydrophilic polymers can be cross-linked to produce hydrogels, whether the polymer is of biological origin, semi-synthetic or wholly synthetic. The hydrogel may be produced from a synthetic polymeric material. Such synthetic polymers can be tailored to a range of properties and predictable lot-to-lot uniformity and represent a reliable source of material that generally is free from concerns of immunogenicity. The matrices may include hydrogels formed from self-assembling peptides, such as those discussed in U.S. Pat. Nos. 5,670,483 and 5,955,343, U.S. Patent Application No. 2002/0160471, and PCT Application No. WO 02/062969.

Properties that make hydrogels valuable in drug delivery applications include the equilibrium swelling degree, sorption kinetics, solute permeability, and their in vivo performance characteristics. Permeability to compounds depends, in part, upon the swelling degree or water content and the rate of biodegradation. Since the mechanical strength of a gel may decline in proportion to the swelling degree, it is also well within the contemplation of the present invention that the hydrogel can be attached to a substrate so that the composite system enhances mechanical strength. In some embodiments, the hydrogel can be impregnated within a porous substrate, so as to gain the mechanical strength of the substrate, along with the useful delivery properties of the hydrogel.

In other embodiments, the pharmaceutical composition comprises a biocompatible matrix made of natural, modified natural or synthetic biodegradable polymers, including homopolymers, copolymers and block polymers, as well as combinations thereof.

Examples of suitable biodegradable polymers or polymer classes include any biodegradable polymers discussed within this disclosure, including but not limited to, fibrin, collagen types I, II, III, IV and V, elastin, gelatin, vitronectin, fibronectin, laminin, thrombin, poly (aminoacid), oxidized cellulose, tropoelastin, silk, ribonucleic acids, deoxyribonucleic acids: proteins, polynucleotides, gum arabic, reconstituted basement membrane matrices, starches, dextrans, alginates, hyaluron, chitin, chitosan, agarose, polysaccharides, hyaluronic acid, poly(lactic acid), poly(glycolic acid), polyethylene glycol, decellularized tissue, self-assembling peptides, polypeptides, glycosaminoglycans, their derivatives and mixtures thereof. Suitable polymers also include poly(lactide) (PLA) which can be formed of L(+) and D(−) polymers, polyhydroxy butyrate, polyurethanes, polyphoshazenes, poly (ethylene glycol)-poly (lactide-co-glycolide) co-polymer, degradable polycyanoacrylates and degradable polyurethanes. For both glycolic acid and lactic acid, an intermediate cyclic dimer is may be prepared and purified prior to polymerization. These intermediate dimers are called glycolide and lactide, respectively.

Other useful biodegradable polymers or polymer classes include, without limitation, aliphatic polyesters, poly (alkylene oxalates), tyrosine derived polycarbonates, polyiminocarbonates, polyorthoesters, polyoxaesters, polyamidoesters, polyoxaesters containing amine groups, poly (propylene fumarate), polyfumarates, polydioxanones, polycarbonates, polyoxalates, poly (alpha-hydroxyacids), poly(esters), polyurethane, poly (ester urethane), poly (ether urethane), polyanhydrides, polyacetates, polycaprolactones, poly (orthoesters), polyamino acids, polyamides and blends and copolymers thereof. Additional useful biodegradable polymers include, without limitation stereopolymers of L- and D-lactic acid, copolymers of bis(para-carboxy phenoxy)propane and sebacic acid, sebacic acid copolymers, copolymers of caprolactone, poly(lactic acid)/poly(glycolic acidypolyethyleneglycol copolymers, copolymers of polyurethane and poly (lactic acid), copolymers of alpha-amino acids, copolymers of alpha-amino acids and caproic acid, copolymers of alpha-benzyl glutamate and polyethylene glycol, copolymers of succinate and poly (glycols), polyphosphazene, poly (hydroxyalkanoates) and mixtures thereof. Binary and ternary systems also are contemplated.

In general, the material used to form a matrix is desirably configured so that it: (1) has mechanical properties that are suitable for the intended application: (2) remains sufficiently intact until tissue has in-grown and healed: (3) does not invoke an inflammatory or toxic response: (4) is metabolized in the body after fulfilling its purpose: (5) is easily processed into the desired final product to be formed: (6) demonstrates acceptable shelf-life: and (7) is easily sterilized.

In another embodiment, the population of cells can be administered by use of a scaffold. The composition, shape, and porosity of the scaffold may be any described above.

Typically, these three-dimensional biomaterials contain the living cells attached to the scaffold, dispersed within the scaffold or incorporated in an extracellular matrix entrapped in the scaffold. Once implanted into the target region of the body, these implants become integrated with the host tissue, wherein the transplanted cells gradually become established.

Non-limiting examples of scaffolds that may be used include textile structures, such as weaves, knits, braids, meshes, non-wovens, and warped knits: porous foams, semi-porous foams, perforated films or sheets, microparticles, decellularized organs or tissues, beads, and spheres and composite structures being a combination of the above structures. Nonwoven mats may, for example, be formed using fibers comprised of a synthetic absorbable copolymer of glycolic and lactic acids (PGA/PLA), sold under the tradename VICRYL sutures (Ethicon, Inc., Somerville, N.J.). Foams, composed of, for example, poly(epsilon-caprolactone)/poly(glycolic acid) (PCL/PGA) copolymer, formed by processes such as freeze-drying, or lyophilized, as discussed in U.S. Pat. No. 6,355,699, also may be utilized.

In another embodiment, the framework is a felt, which can be composed of a multifilament yarn made from a bioabsorbable material. The yarn can be made into a felt using standard textile processing techniques consisting of crimping, cutting, carding and needling. In another embodiment, cells are seeded onto foam scaffolds that may be used as composite structures.

The framework may be molded into a useful shape, such as to fill a tissue void. The framework can therefore be shaped to not only provide a channel for neural growth, but also provide a scaffold for the supporting and surrounding tissues, such as vascular tissue, muscle tissue, and the like. Furthermore, it will be appreciated that the population of cells may be cultured on pre-formed, non-degradable surgical or implantable devices.

Pharmaceutical compositions may include one or more trophic factors, e.g., survival factors, growth factors, and the like, to supplement and/or further differentiate the delivered cells. In some embodiments, the one or more trophic factors is suspended within the carrier. In other embodiments, the one or more trophic factors is associated with a gel, e.g., a biocompatible and/or biodegradable polymer, such as poly(lactic acid), poly(lactic acid-co-glycolic acid), methylcellulose, hyaluronic acid, collagen, and the like, or a scaffold, as disclosed herein or as known in the art.

The skilled artisan can readily determine the number of cells and optional additives, vehicles, and/or carrier in compositions and to be administered in methods of the invention. Typically, any additives (in addition to the active cell(s)) are present in an amount of 0.001 to 50 wt % solution in phosphate buffered saline, and the active ingredient is present in the order of micrograms to milligrams, such as about 0.0001 to about 5 wt %, including about 0.0001 to about 1 wt %, including about 0.0001 to about 0.05 wt % or about 0.001 to about 20 wt %, including about 0.01 to about 10 wt %, and including about 0.05 to about 5 wt %. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse: and, the dosage of the composition(s), concentration of components therein and timing of administering the composition(s), which elicit a suitable response.

Other factors can be included and/or administered prior to, after or concomitantly with cells.

For example, factors that promote cell migration can also be administered prior to, after or concomitantly with cells. Factors promoting cell migration include, but are not limited to, Pepstatin A, NGF, BDNF and/or GDNF.

Factors that decrease apoptosis can also be beneficial in connection with the present disclosure. Factors that decrease apoptosis include, but are not limited, Rho-kinase inhibitor Y-27632 (ROCK), to (3-blockers, angiotensin-converting enzyme inhibitors (ACE inhibitors), AKT, HIF, carvedilol, angiotensin II type 1 receptor antagonists, caspase inhibitors, cariporide, and eniporide.

Exogenous factors (e.g., cytokines, differentiation factors (e.g., cellular factors, such as growth factors or angiogenic factors that induce lineage commitment), angiogenesis factors and anti-apoptosis factors) can also be administered prior to, after or concomitantly with cells. Doses for administration(s) are variable and may include an initial administration followed by subsequent administrations.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. According to the present invention, however, any vehicle, diluent, or additive used would have to be compatible with the cells.

Injectable solutions can be prepared by incorporating the cells utilized in practicing the present invention in the required amount of the appropriate solvent with various amounts of the other ingredients, as desired.

In some embodiments, examples of compositions comprising the cells of the invention include liquid preparations for administration by direct/local injection. Such compositions may be in admixture with a suitable carrier, diluent, or excipient such as sterile water, physiological saline, glucose, dextrose, or the like. The compositions can contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 23rd edition, 2020, incorporated herein by reference, may be consulted to prepare suitable preparations, without undue experimentation.

Solutions, suspensions and gels normally contain a major amount of water (e.g., purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose), may also be present. The compositions can be isotonic, i.e., they can have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes.

A pharmaceutically acceptable preservative or cell stabilizer can be employed to increase the life of the compositions. If preservatives are used, it is well within the purview of the skilled artisan to select compositions that will not affect the viability or efficacy of the cells as described in the present invention.

### Methods of Administration and Treatment

According to another aspect of the present disclosure, there are provided methods for treating an enteric neuropathy comprising administering stem cell-derived ENCCs or a pharmaceutical composition comprising stem cell-derived ENCCs to the intestine of a subject in need thereof, particularly to the muscular wall of the intestine.

The cells and compositions prepared as described herein can be administered to a subject by a variety of methods available to the art. In some embodiments, the cells and compositions are administered by localized injection.

In some embodiments, cells (or pharmaceutical compositions comprising the cells) are administered by localized injection to two or more, three or more, four or more, or five or more locations of the intestine. In other embodiments, cells are administered by bilateral injections about every 1-3 centimeters in the muscular wall of the intestine, such as an aganglionic intestine. In other embodiments, cells are administered about every 2 centimeters in the muscular wall of the intestine, such as an aganglionic intestine.

The quantity of cells to be administered will vary for the subject being treated. In one embodiment, each dose of cells administered comprises about 1×10⁵ to 1×10⁹ cells. In one embodiment, between about 10⁴ to 10⁸, including about 10⁵ to 10⁷, such as about 3×10⁷ stem cells can be administered to a subject, e.g., a human subject. However, the precise determination of what would be considered an effective dose may be based on factors individual to each patient, including their size, age, disease or injury, size damage, amount of time since the damage occurred and factors associated with the mode of delivery.

Suitable regimes for initial administration and further doses or for sequential administrations also are variable, may include an initial administration followed by subsequent administrations.

In some embodiments, the cells are ENCCs that are an allogeneic iPSC-derived enteric neural crest progenitor therapy that can be delivered by injection into the muscular wall of the intestine in patients with, for example, Hirschsprung Disease (HD) or other enteric neuropathies.

In more particular embodiments, the enteric neuropathy is the total intestinal aganglionosis form of HD or long segment forms of HD. In other particular embodiments, the enteric neuropathy is aganglionosis of the rectosigmoid colon (short segment disease), aganglionosis of the sigmoid colon (long segment disease), congenital aganglionosis in Hirschsprung disease (HD), autoimmune-mediated loss of neuronal subtypes in esophageal achalasia and Chagas disease, degenerative neuropathies in chronic intestinal pseudo-obstruction or gastroparesis, enteric neuropathy caused by nerve agents (e.g., Gulf War syndrome) and/or diabetic gastroparesis.

In other embodiments, the dosing form is a thaw and inject formulation, with little to no requirement for further manipulation at the clinical site (e.g., removal of excess supernatant from cells post-thaw). In another embodiment, route of administration will be via a minimally invasive surgical approach if possible, with intestinal injections delivered via endoscope or laparoscope unless open surgery has been or is required, in which case direct injection will occur.

In some embodiments, standardized intestine injection (SII) will deliver cells intramuscularly in divided doses each (e.g., of about 1×10⁷ cells) in bilateral injections every two centimeters for up to two, four, six, eight, ten, twelve, fourteen, sixteen, eighteen, twenty, twenty-two etc. doses in the aganglionic intestine.

In some embodiments, a 21-25-gauge needle or endoscopic equivalent is used for administration of the cells.

In some embodiments, the doses are administered using ENCCs suspended to a concentration in which each injection is 0.25-2 mL.

In some embodiments, injection can occur under direct visualization with the needle held in place for thirty seconds after raising a bleb for cell delivery.

In some embodiments, the subject in need thereof has been immunosuppressed (by methods available to an art worker) prior to administration of the ENCCs derived from stem cells.

In some embodiments, the methods involve dividing the intestine prior to injection and putting up a stoma and mucus fistula.

In some embodiments, the injections are made serially along the intestinal wall.

In some embodiments, the injections are made in the stomach, the aganglionic esophagus, the aganglionic anal sphincter, or another region of the intestine.

The methods described herein, in some embodiments, enable rescuing intestinal function in a subject suffering from an enteric neuropathy. In other embodiments, the method enables rescuing anal sphincter or esophageal function in a subject (as in Chagas disease etc) with injection.

In some embodiments, the ENCCs administered to the subject are in the form of neurospheres (large cellular aggregates).

In some embodiments, the ENCCs administered to the subject comprise a substantially homogeneous population of cells. In other embodiments, the ENCCs comprise a heterogeneous mixture of distinct cell populations.

In some embodiments, the ENCCs or neurospheres thereof undergo additional maturation and/or differentiation in vivo following their administration.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate certain embodiments of the present disclosure and are not intended to limit the scope of the invention in any way. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### EXAMPLE 1

### Evaluation Of Advanced Stem Cell Enteric Neuropathy Therapy (Ascent)

iPSC Starting Material

The LiPSC-GR1. 1 cell line was generated under cGMPs at Lonza's Cell Therapy manufacturing facility from human cord blood. The LiPSC-GRI. 1 cell line was reprogrammed from male CD34 cells with a non-integrating vector under GMP (1,2). CD34 cells were isolated from cord blood by Miltenyi Biotec's CliniMACS system. The CD34 cells were reprogrammed by GMP grade episomal plasmids containing Oct4, Sox2. KLF4, c-Myc, Lin28, pEb-Tg. iPSC clones were selected, expanded and characterized. The human iPSCs manufactured under cGMP conditions fulfilled the main characteristics of pluripotent stem cells and passed standard safety assays, including plasmid clearance, karyotype, STR, sterility, mycoplasma, and endotoxin tests.

### Production of ENCCs

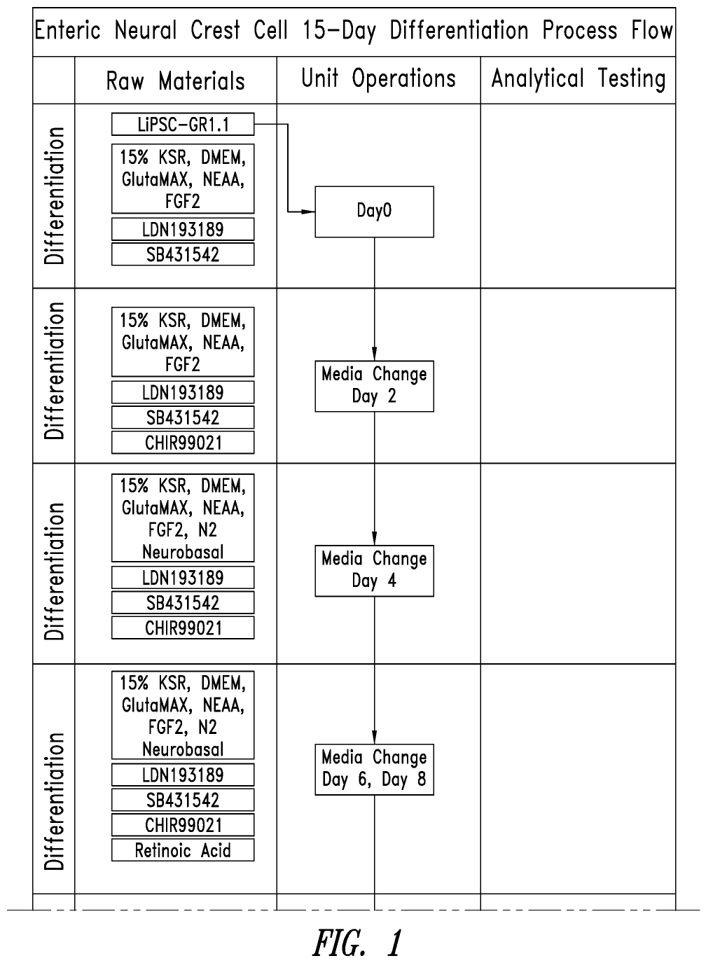

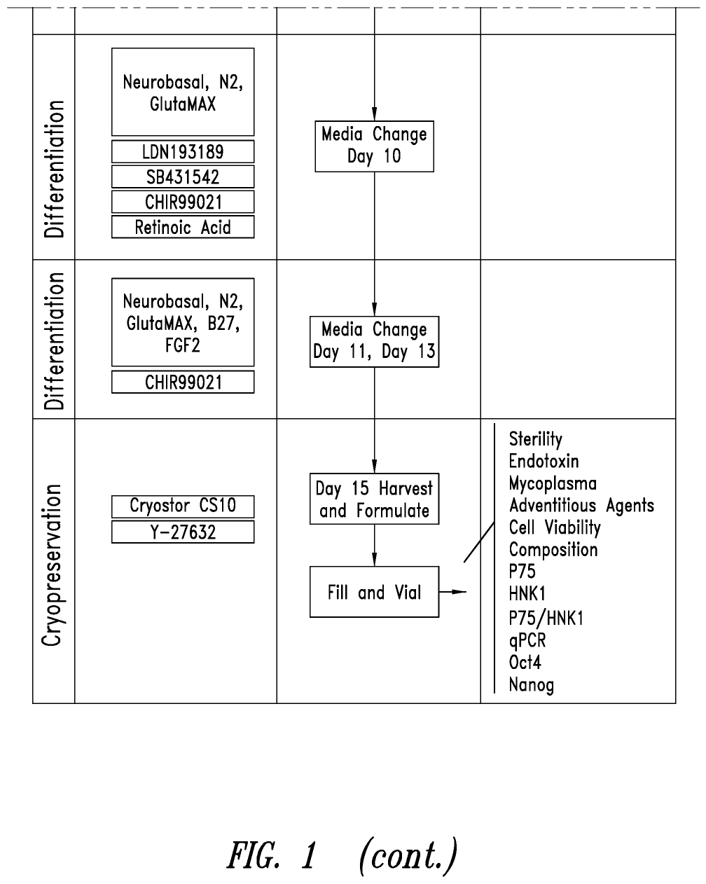

The process of differentiation to ENCCs is a 15-day dual-SMAD-inhibition protocol. Lineage specificity is directed by the small molecules LDN193189 and SB431542 to inhibit the two branches of the SMAD pathway, CHIR99021 to activate the WNT pathway, followed by fibroblast growth factor 2 (FGF2) and retinoic acid to further direct specification and expression of vagal and early enteric nervous system markers (FIG. 1) (8, 9: discussed below).

### Neurosphere Formation

To identify the ideal range for neurosphere formation, ASCENT was cultured in suspension at different speeds both on a rocker and in spinner flasks. In T-flasks on a rocker, it was determined that a range of 8-30 rpm was sufficient to differentiate ASCENT, and that in spinner flasks, a range of 30-70 rpm was able to generate ASCENT. This range in speed for each culture system allows the manufacturing of ASCENT to be a tunable process and builds in flexibility to accommodate different cultures, systems, and equipment.

In adherent cultures, it was determined that the cells can be lifted into suspension between 9 and 12 days of culture and still generate ASCENT successfully.

### Formulation of ASCENT

Final differentiation was initiated in T225s on a rocker set to 11 rpm at 37° C./5% CO₂ in knockout serum replacement (KSR) medium (KO DMEM+15% KSR (ThermoFisher, 10828-028), 1X GlutaMAX Supplement (ThermoFisher, 35050061), 1X Non-Essential Amino Acids (ThermoFisher, 11140-050), LDN193189 (100 nM, Stemgent, Cambridge, MA), and SB431542 (10 uM, Tocris, Ellisville, MI). The media is changed every 48 hours. Day 0 of differentiation is the day the medium is switched from mTeSRI medium to KSR medium containing LDN193189 and SB431542. CHIR099021 (3 μM, Tocris, 4423) is added to the differentiation media on days 2 through day 15. From day 4 through day 10 as described previously (7), the KSR medium was gradually replaced with increasing amounts of Neurobasal medium supplemented with GlutaMAX Supplement, 1X N2 supplement (ThermoFisher, 17502001) (NB/N2 medium). ENCC differentiation involves additional treatment with Retinoic Acid (10 μM, Sigma-Aldrich, R2625-50mg) from day 6 through day 11. The differentiated cells may be sorted for CD49D at day 11. Days of differentiation in text and figures refer to the number of days since the pluripotent stage (day 0).

On days 11 through 15, the ENCCs were cultured in NB/N2 medium supplemented with 1X B27 Supplement (ThermoFisher, 17504044) containing CHIR99021 and FGF2 (10 nM, R&D Systems, 233-FB-001MG/CF). ENCCs were harvested and cryopreserved on day 15 of the differentiation process in Cryostor CS10 and 10 μM Y-27632. The ENCCs were cryopreserved as neurospheres. A fresh sample was also taken for testing.

### Characterization of ENCCs

Characterization of ENCCs was accomplished through extensive in vitro analysis including immunofluorescence staining, flow cytometry, and qPCR. Immunostaining confirmed the expression of neural crest markers and the lack of expression of pluripotent markers. Flow cytometry analysis of p75 and HNK1(6) confirmed enteric neural crest specific markers on Day 15. Gene expression of Oct4 and Nanog on Day 15 ENCC were analyzed by qPCR, as well as additional ENCC markers to ensure the cells were directed to the neural crest pathway.

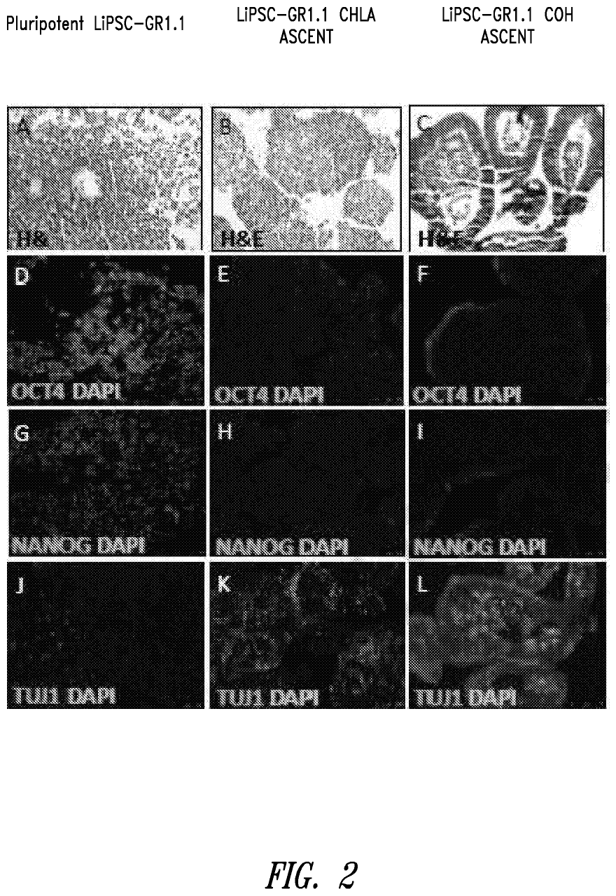

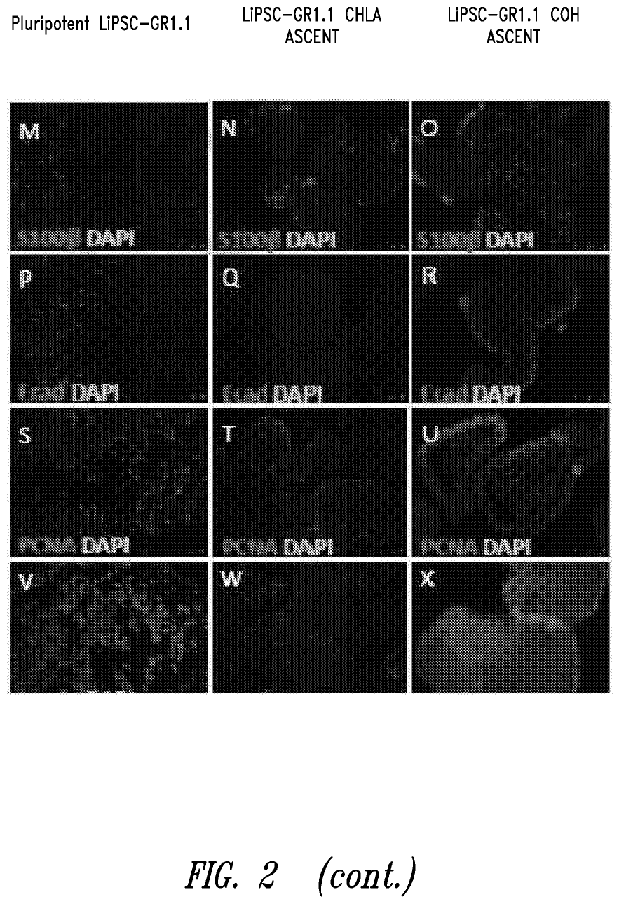

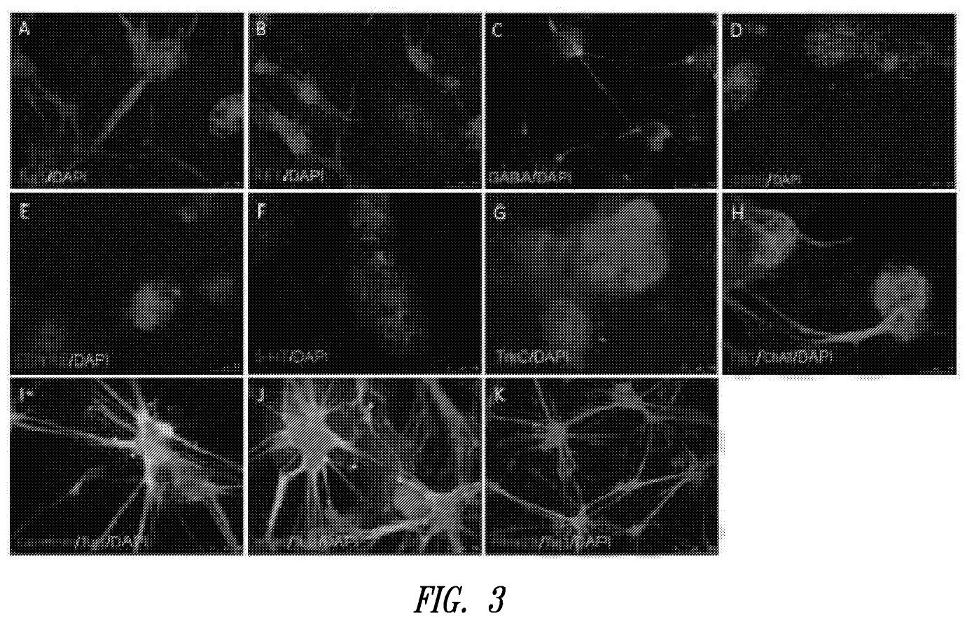

Immunofluorescent (IF) analysis and was accomplished on Day 0 pluripotent LiPSC-GR1.1 cells and ENCCs (ENCCs at D15 of differentiation; two times (CHLA ASCENT column and COH ASCENT column: FIG. 2) to determine the expression of neural marker Tuj1. glial marker S100B, neuroepithelial marker E-cadherin, proliferating cell nuclear antigen (PCNA) and human marker Lamin. In all cases, robust expression of Tuj1 and PCNA expression was identified in ENCCs. Immunofluorescent detection of pluripotent stem cell markers Oct4 and Nanog of the ENCCs compared to LiPSC-GR1.1 in its pluripotent state confirmed that ENCCs have no expression of pluripotent markers after the differentiation process. Similar expression was observed in cryopreserved cells.

To confirm ENCCs can further differentiate into the cellular subtypes of the ENS, ENCCs were differentiated in vitro for an additional 25 days in media containing glial cell-derived neurotrophic factor (GDNF) and ascorbic acid (FIG. 3). Specifically expressed by the gut mesoderm, GDNF is part of the TGF-β super family has been implicated in the proliferation, differentiation, migration, and survival of enteric neural crest cells during ENS development (10, 11). The cell surface of migrating ENCCs express a GDNF receptor complex composed of Ret and co-receptor GDNF family receptor 1a (GFRa1), a binding complex that has been shown to influence differentiation of neural progenitor cells (12). Ascorbic acid has been shown to increase differentiation to dopaminergic and serotonergic cell fates (13).

Immunostaining revealed that ENCCs are of the enteric lineage (TrkC/RET/EDNRB-positive) and differentiated into excitatory neurons (ChAT-positive), inhibitory neurons (GABA-positive), and glia (SOX10/s100β-positive) (FIG. 3). Abundant Tuj1-positive neurons and SOX10/GFAP-positive and SOX10/s 100β-positive glia are identified in the culture system. Tuj1-positive neurons retain their vagal identity by expression of TrkC, RET, and EDNRB. Enteric neurons express excitatory (ChAT) as well as sensory neuron marker serotonin (5HT).

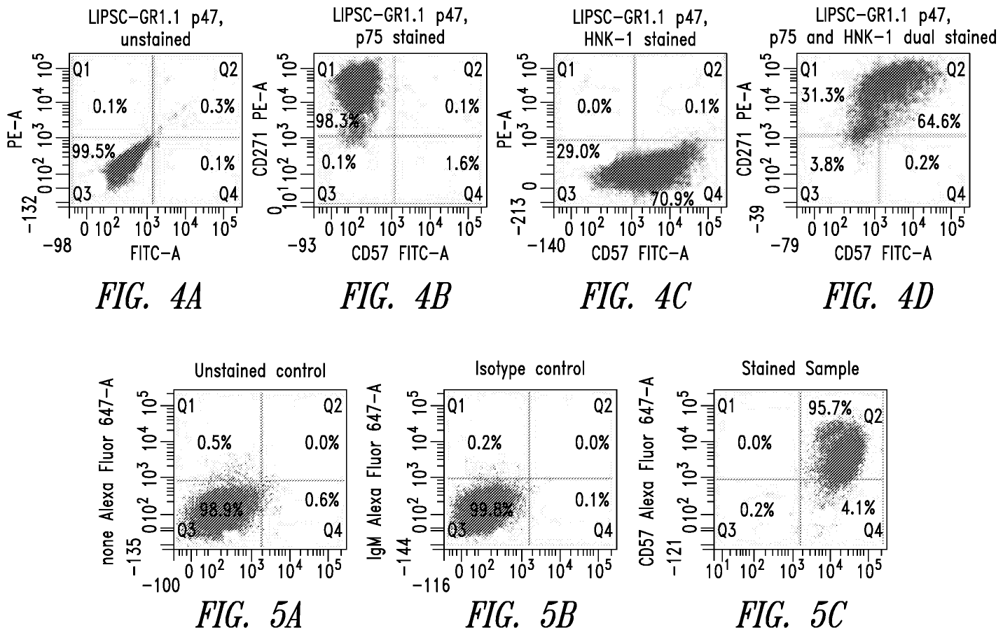

Flow cytometry detects high expression of HNK1⁺ and p75⁺ cells on Day 15 (FIG. 4). HNK1 and p75, also known as CD57 and CD271 respectively, are both established markers of enteric neural crest cells (12). Cells are analyzed for high expression of the two markers individually, as well as co-expression levels.

HNK1 and p75 expression of D15 fresh ENCC was measured by flow cytometry and compared with an unstained control and isotype control. The unstained control determines the inherent autofluorescence levels and sets the gates and voltage (FIG. 5). Isotype controls establish the level of any non-specific background signal caused by primary antibodies (FIG. 5). Both controls show that there is little to no autofluorescence or non-specific background with these antibodies. Flow analysis of ENCC indicates that the differentiation protocol yields greater than 90% p75⁺HNK1⁺ cells (FIG. 5). Similar expression was observed in cryopreserved cells.

Prior to being adapted to suspension culture, ENCC was cultured in adherent cultures. LiPSC-GR1.1 cells were expanded and grown on 6-well plates (Corning, Cat 3471) until approximately 70% confluency. When the desired confluency was achieved, ENCC was differentiated in the same way as described above, with the same media formulations and 48-hour media changes until day 11. At day 11, ENCC was lifted into suspension culture on Ultra-low Attachment plates (Corning, cat 140675) in day 11 media, and left in static suspension culture until day 15, with media changes every 48 hours. The cells were then used for experiments or cryopreserved in CryoStor CS10 with or without 10 μM Y-27632.

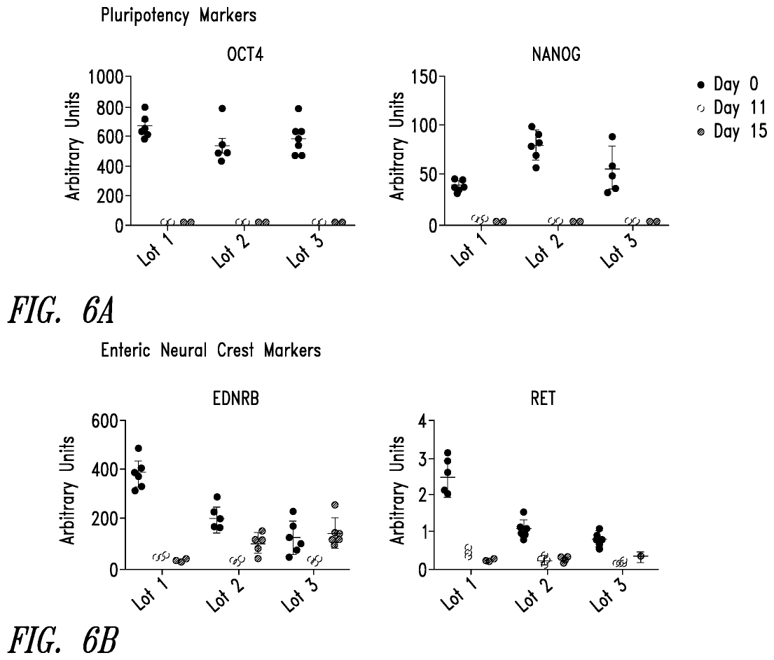

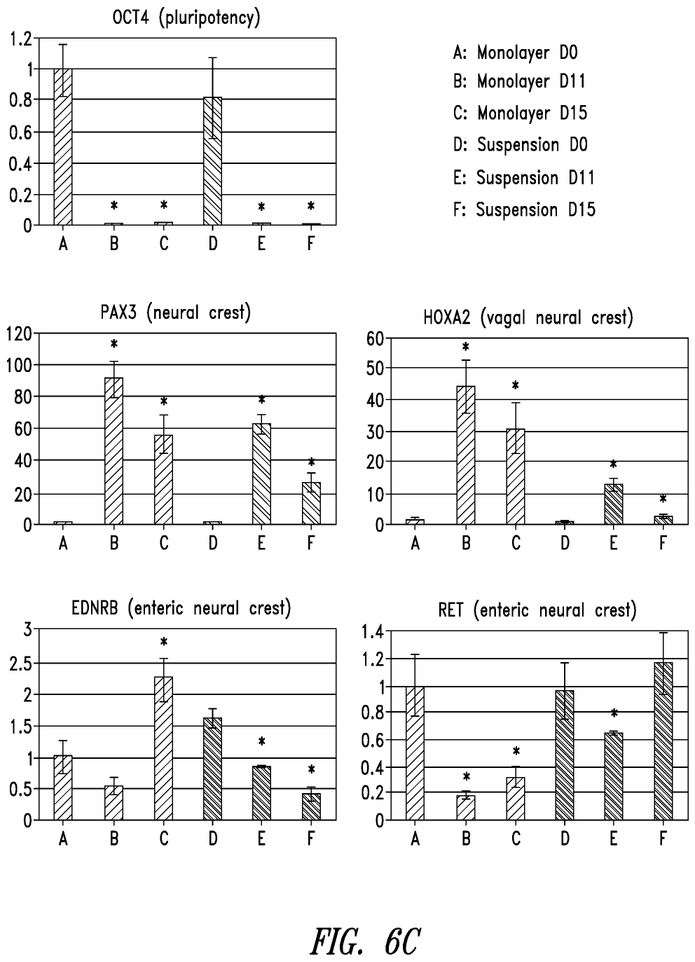

To determine reproducibility and consistency of the ENCC differentiation protocol, adherent ENCC was differentiated in three separate laboratories. ENCC derived from each laboratory were then analyzed by the each of the laboratories to compare the three unique lots by qPCR analysis for reproducibility of the protocol and results. Samples were taken at Day 0, Day 11, and Day 15 of differentiation (FIG. 6A and B). Pluripotency markers demonstrated, as expected, high expression at Day 0, and markedly decreased expression to Day 15. Enteric neural expression of the three lots follow the same pattern, showing high expression in the pluripotent state, decreasing levels to day 11, and increasing again by day 15.

Once reproducibility of adherent ENCC was confirmed, a study was executed to determine if ENCC differentiates in suspension culture compared to adherent culture. Gene analysis by qPCR of comparing ENCC differentiated in adherent and suspension cultures demonstrates the expected gene expression profile (FIG. 6C). Pluripotency marker Oct4 decreases over time, while neural crest marker PAX3, and vagal neural crest stem cell marker HOXA2 increases then decreases as the differentiation progresses. Enteric neural crest cell markers EDNRB and RET increase over the differentiation time.

Gene expression analysis by qPCR was conducted on both the fresh and cryopreserved lots of ENCC harvested on Days 11 and 15 of differentiation and placed in

RNAlater (Life Technologies, catalog number AM7021) for preservation and later processing. The RNA was extracted using the Qiagen RNeasy kit and run using the LightCycler® 480 SYBR Green I Master (Roche, catalog number: 5102413001). All samples were normalized to the GAPDH housekeeping gene then compared to Day 0 pluripotent LiPSC-GR1.1 cells.

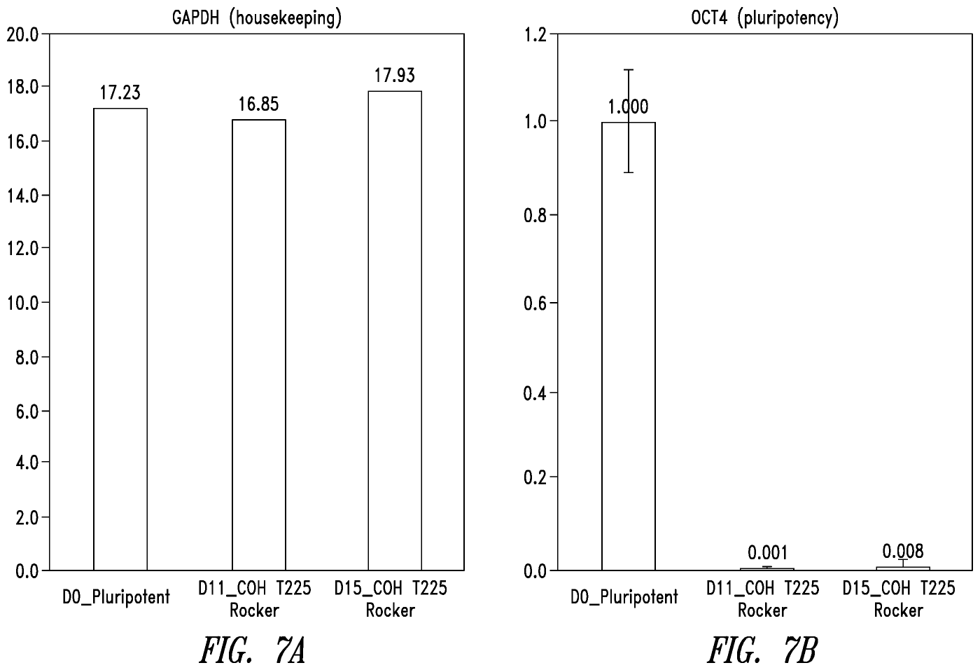

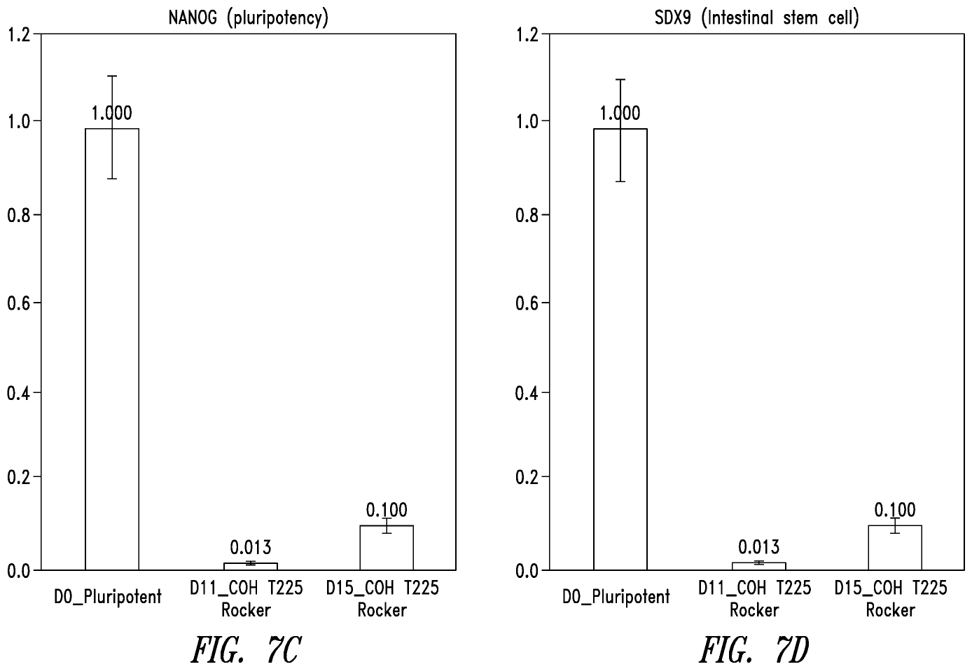

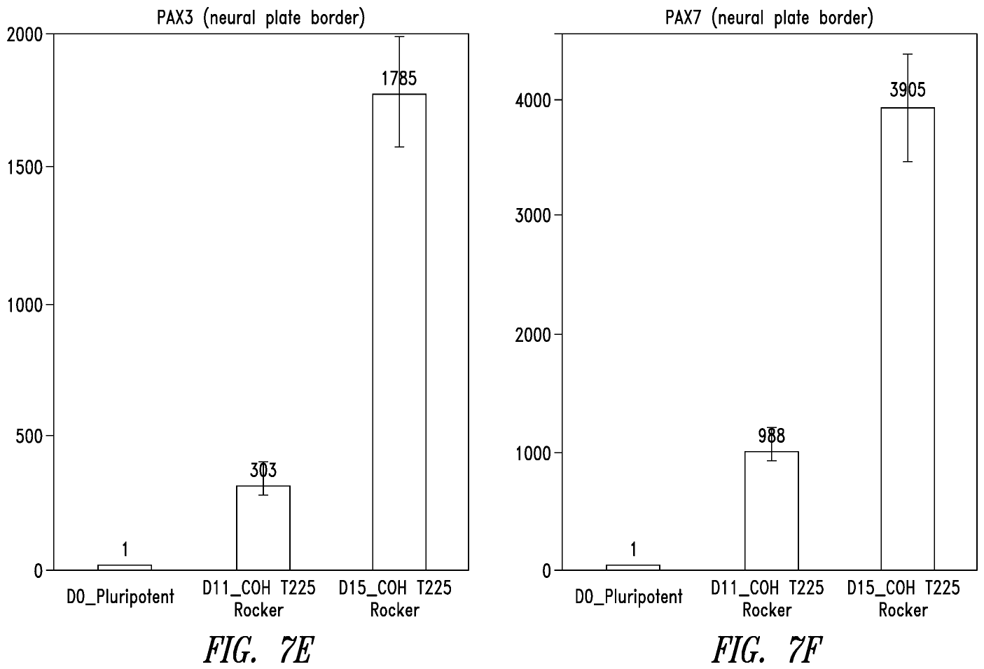

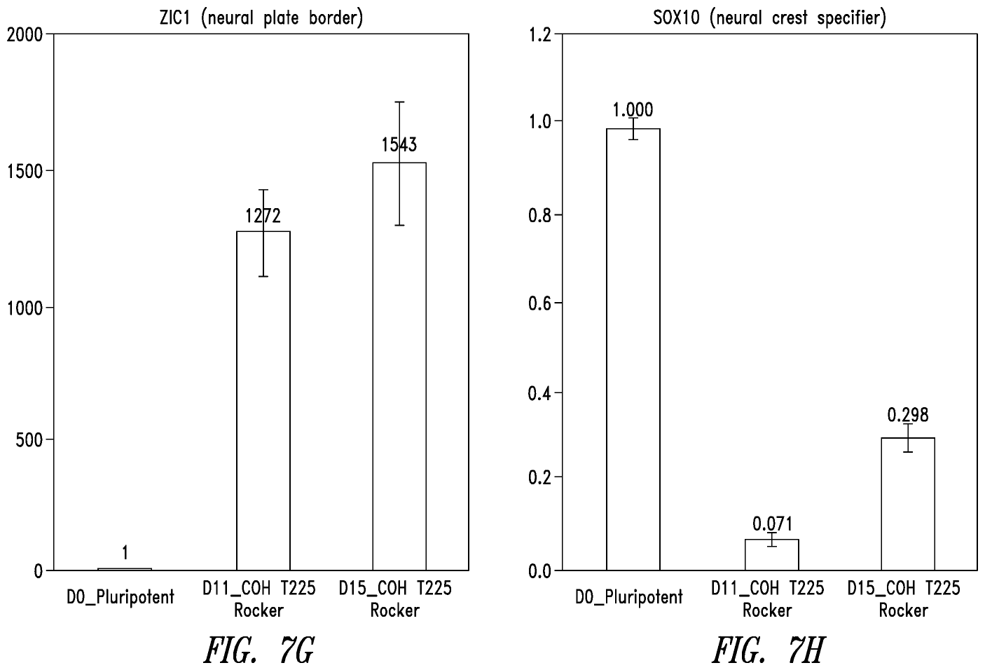

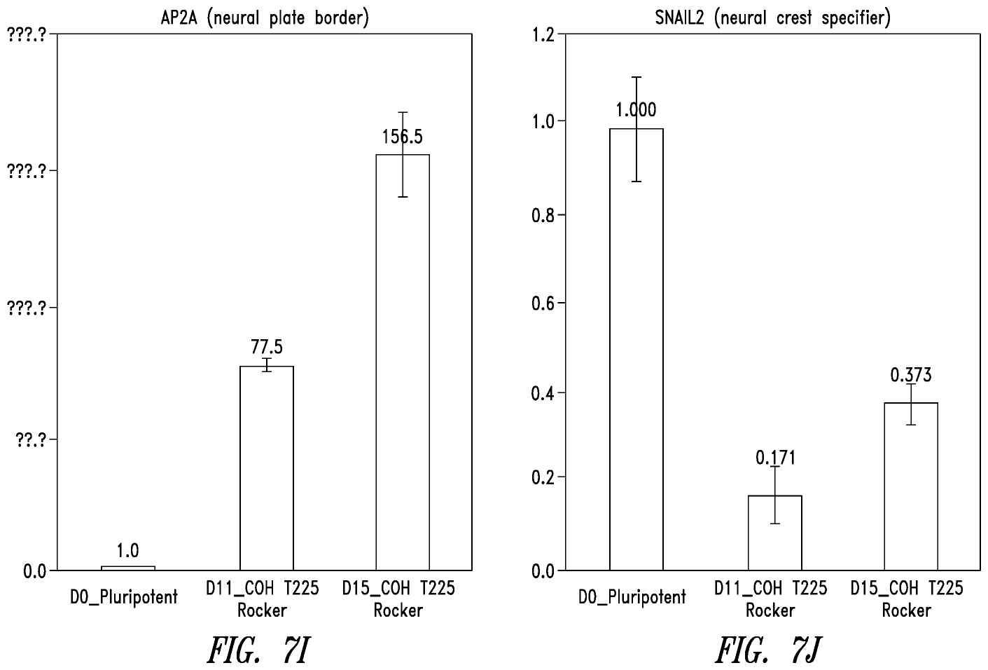

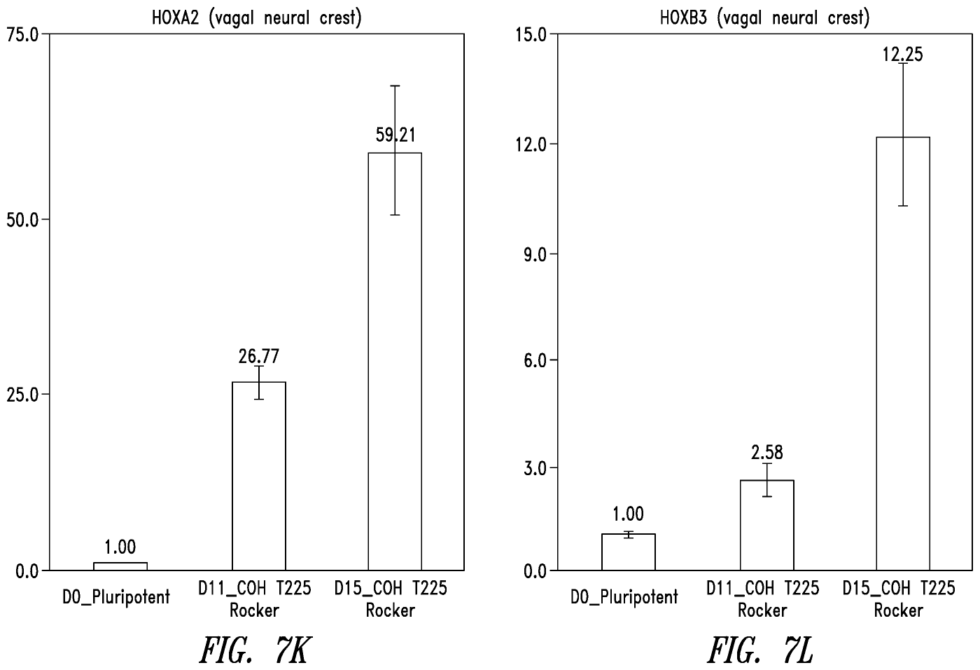

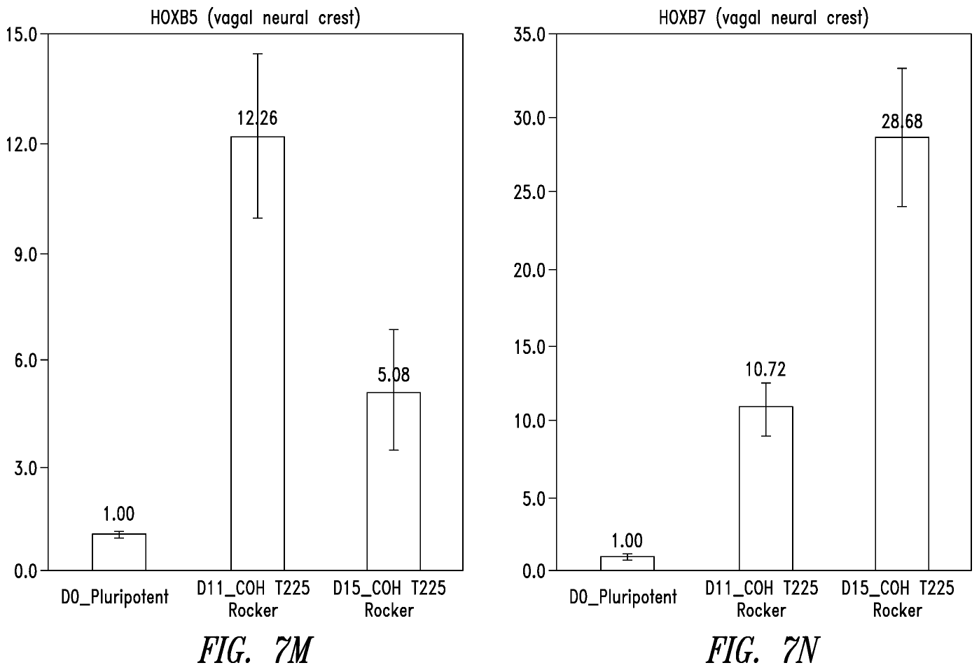

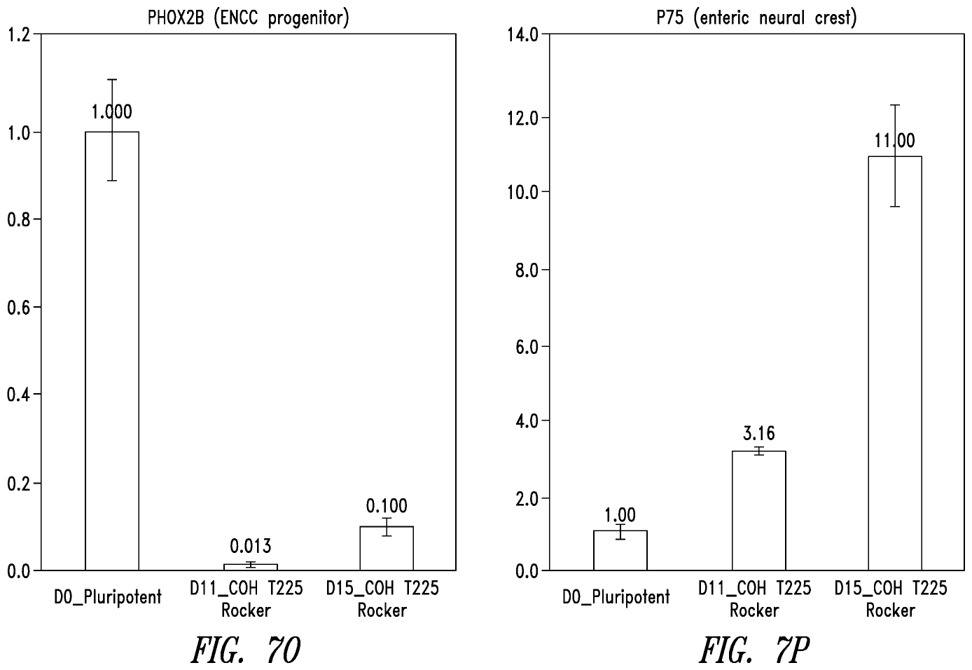

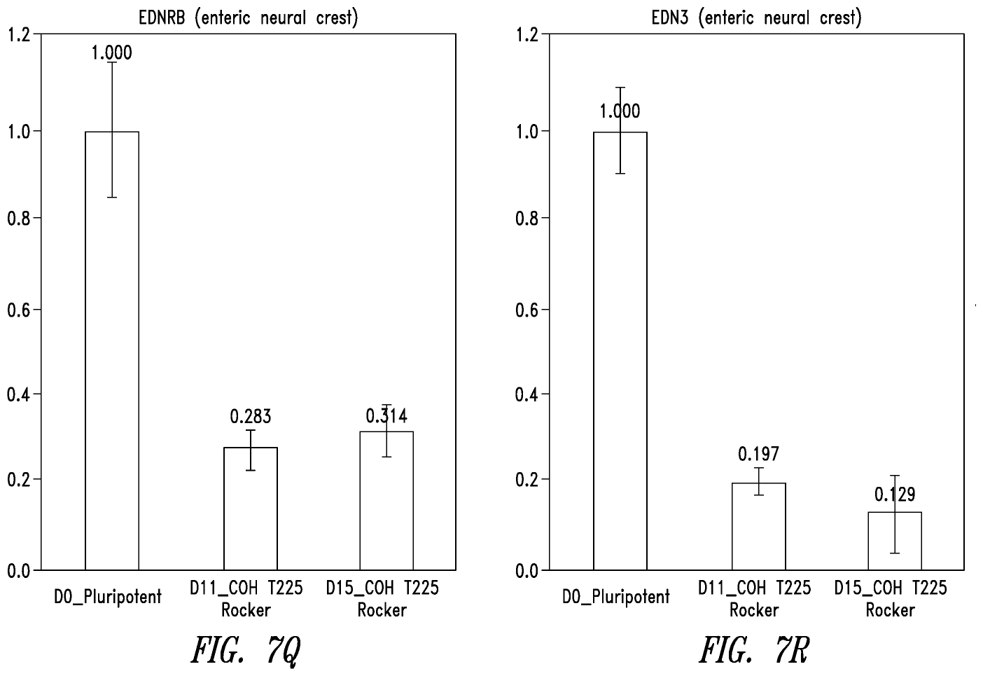

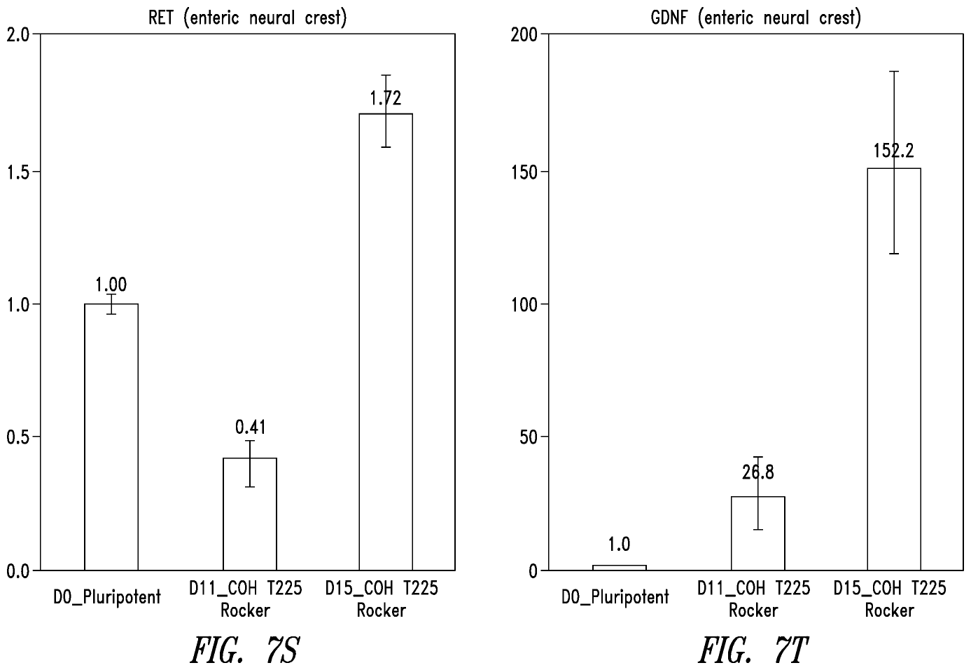

Results of the qPCR analysis of fresh ENCC (FIG. 7) show no expression of pluripotency markers Oct4 and Nanog at Days 11 and 15 for all conditions. Neural crest cell gene expression of Paired box protein Pax-3 (Pax3) and Transcription factor SOX-10 (Sox10) are increased at Day 11 and decreased at Day 15. Vagal neural crest markers Homeobox protein Hox-A2 (HOXA2) is upregulated at day 11 and day 15, while the rest of the HOX genes have less robust expression with p75. Proto-oncogene tyrosine-protein kinase receptor Ret (RET), and GDNF increasing in expression, and Endothelin receptor type B (EDNRB) and its ligand Endothelin-3 (EDN3) decreasing between day 11 and day 15. Similar expression was observed in cryopreserved cells.

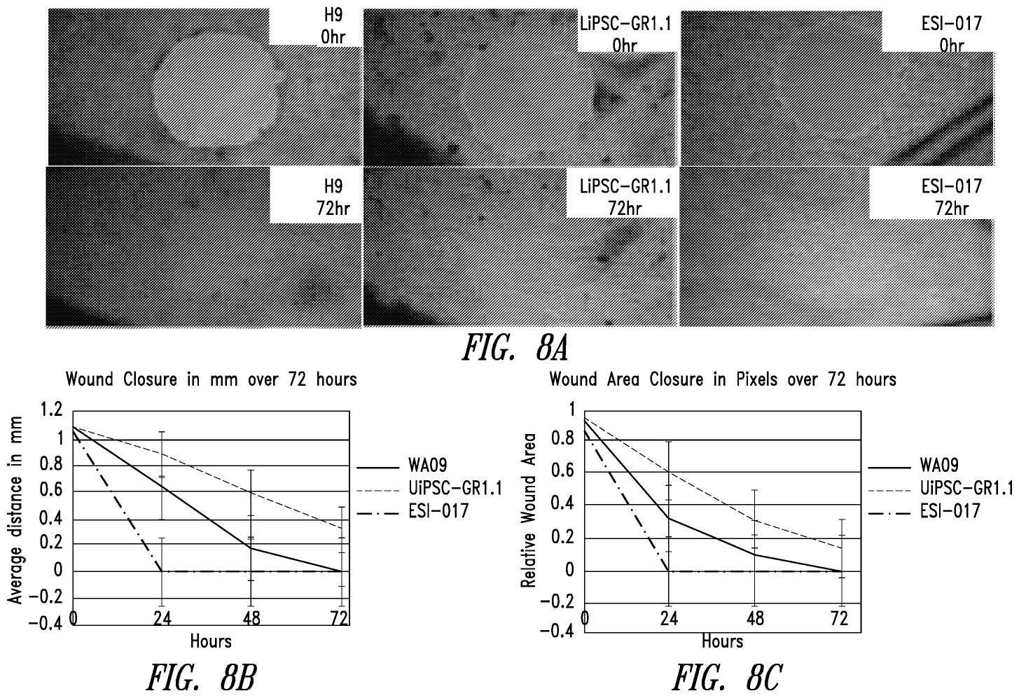

A key aspect of therapy with ENCC is the ability for the cells to migrate from the injection site and through the intestinal wall to repopulate the ENS as the network of cells is absent throughout the intestinal muscle layer in the conditions termed enteric neuropathies. To determine cell migration ability and rate of ENCC in vitro, a modified scratch assay model was performed that employs a drill press to make symmetrical and reproducible wounds and compared it to wildtype human embryonic stem cell lines H9 (WA09) (WiCell) assessed in previous scratch assay studies (8), and ESI-017 (BioTime, Inc). An integrated computerized calculation of the area of the wound measures the rate of migration. Cells were plated in 6-well plates at a density of 7×10⁶ cells per well to achieve confluence during an overnight incubation. Eight wounds per well were created using a drill press and observed until wound closure with measurements recorded every 24 hours. Excellent migration was documented for ENCC derived from iPSC (FIG. 8).

When attempting in vivo studies with ENCC, it became evident that the animal disease models of Hirschsprung disease (16) or other enteric neuropathies were too fragile to be effective and most die within the first month of life, precluding study of migration or cell survival. The further addition of immunosuppression in order to implant human donor cells resulted in no survivors. Investigations were therefore carried out in immunosuppressed murine models (NOD/SCID gamma chain deficient (NSG) and nude rats), immunosuppressed large animals (Yucatan mini pigs) and co-implantation with iPSC-derived intestine (HIO-TESI). HIO-TESI, when not supplemented with ENS precursors results in growth of intestine that contains all the epithelial and mesenchymal attributes of native intestine, but lacks an ENS.

There are a number of Hirschsprung Disease (HD) knockout mice models available, but the most commonly studied mouse models for HD are animals with transgenic alterations of endothelin receptor type B (Ednrb). Not all of the models above have reliable large intestine enteric neuropathies and even Ednrb models can be quite variable, with completely different amounts of intestine affected even in littermates, which may cloud in vivo results in which the effect of therapy is then being studied in a variable background. In other models, determining the effect of transplanted cells is difficult since the native ENS presence can be nearly normal (most commonly in the survivors who can tolerate possible ENS cellular therapies, rendering rescue effects unknown) or, conversely, so severe that the new born mouse cannot survive. The least variable Ednrb model, and one that has some survivors of neonatal surgery in other studies is the B6. 129S7-Ednrb™1Ywa/FrykJ (17) mouse hereafter termned Ednrb -/-, housed at Jackson Laboratory (stock no. 021933). This model is an Ednrb gene knock-out mutant, where exon 3 has been replaced by a neomycin resistance cassette (18). These mice have disrupted neural crest cell development and, like patients with Hirschsprung disease, can have failure of migration of enteric nervous system components in the distal intestine. Although still variable, of the available mouse models this model has the most consistency in generating survivable but meaningful segments of aganglionic intestine, and tissue engineered intestine was generated from these segments (19). However, although there are reports of successful colon surgery in these animals (20), we were unable to generate enough survivors to an age where it would be feasible to deliver ENCC once we added the required immunosuppression with cyclosporine.

As the transgenic mouse model was not reliable, alternative models of aganglionosis were used. An alternate disease model for determining the efficacy of ENCC is to co-implant ENCC with human intestinal organoids (HIO) generated from human pluripotent stem cell lines (21). HIOs are derived from the differentiation of pluripotent stem cells into all of the components of the small intestine, and they always exclude any components of the ENS. Two lines were compared for this model: embryonic stem cell line WA0922 (known as H9) and a PHOX2B knockout induced pluripotent stem cell line (23) (hereafter referred to as PHOX2B-/-) as an additional disease model. These models allow more biological replicates to be generated and analyzed. Briefly, HIO-TESI is generated from the transplantation of a biodegradable scaffold seeded with iPSC that have been differentiated for 35 days into intestinal precursors, into a vascularized space in the mouse. This is a well-established protocol (14, 24). Briefly, hPSCs were treated with defined factors to produce HIOs (21). Independently, hPSCs were differentiated into ENCC. PGA/PLLA scaffolds were seeded with 4-6 HIOs (day 35 of differentiation) and approximately 1×10⁶ cells of ENCC in 30 uL (day 15 of differentiation), implanted into the omentum of NSG mice, and allowed to mature for 3 months prior to explantation. For both cell lines, the HIOs were co-implanted with day 15 fresh ENCC into the omentum of NOD/SCID mice for a minimum of 6 and maximum of 12 weeks. ENCC integrates and migrates through the HIO scaffold and creates functional neurons in appropriate locations.

In order to demonstrate the feasibility and utility of HIO-TESI to understand cellular proliferation and migration of ENCC, several studies were performed, as described below.

H9 hESCs were directed into HIOs and expanded in culture for 28 to 35 days, as described previously (24).

Concurrently, ENCC was generated. There is high survival of ENCC in ENCC-HIO-TESI when the HIO are generated from H9 cells as well as a further genetic variant HIO type, PHOX2B-/-, that correlates to a human enteric neuropathic condition.

It was previously shown that implanted tissue-engineered constructs grow from about 80-85% of all implantations whether derived from adult stem cells or iPSC (24, 25). As expected, approximately 20% of all HIO-TESI implantations did not result in the growth of TESI when implanted. TESI implantation is technically complex and relies on delivering cells differentiated over 35 days on a biodegradable scaffold in a survival surgery with subsequent injections for pain control. In the constructs that did develop, multiple alternative strategies were pursued in order to verify that fresh ENCC differentiates into all of the relevant cell types in the correct histologic locations within the intestine and with limited biodistribution outside of the site of delivery.

Investigations that support these conclusions include identification of robust differentiated cells that resulted from the addition of ENCC to ENCC-HIO-TESI derived from H9 and PHOX2B-/- HIO, and a delayed model of injection which more closely approximates the eventual human therapy.

ENCC-HIO-TESI derived from H9 and PHOX2B-/- HIO and LiPSC-GR1.1 ENCC host differentiated networks of ENS cells that derive from the donor ENCC product.

The experimental protocol was established to generate ENCC-HIO from WA09 (H9) cells (previously described), but these experiments were expanded with a cell line disease model with a mutation in the PHOX2B-/- gene (1) from which HIO was generated, in order to show that ENCC may migrate in aganglionic intestine that forms with the same mutation in which an enteric neuropathy might be expected. Because children who will receive ENCC can have a systemic genetic mutation, it need to be determined that EENC could migrate in analogous conditions. Migration and differentiation were found in all conditions. HIO derived from the PHOX2B gene CRISPR-knockout iPSC line (PHOX2B-/-) (23) were co-implanted with ENCC.

ENCC-HIO-TESI generated from H9 HIO and LiPSC-GR1.1 ENCC demonstrates appropriately located differentiated components of the ENS. After successfully demonstrating neuronal and glial engraftment in ENCC-HIO-TESI, retention of ENCC markers and neuronal subtypes was evaluated. Well-organized PHOX2B-/- and TRKC/RET/EDNRB triple-positive ganglia were identified throughout the submucosal and myenteric plexuses similar to human fetal intestine. The ENS contains numerous unique subtypes of neurons with distinct functions, electrophysiological properties, and neurotransmitter expression. Myenteric neurons include excitatory and inhibitory motor neurons, descending and ascending interneurons, and intrinsic primary sensory neurons.

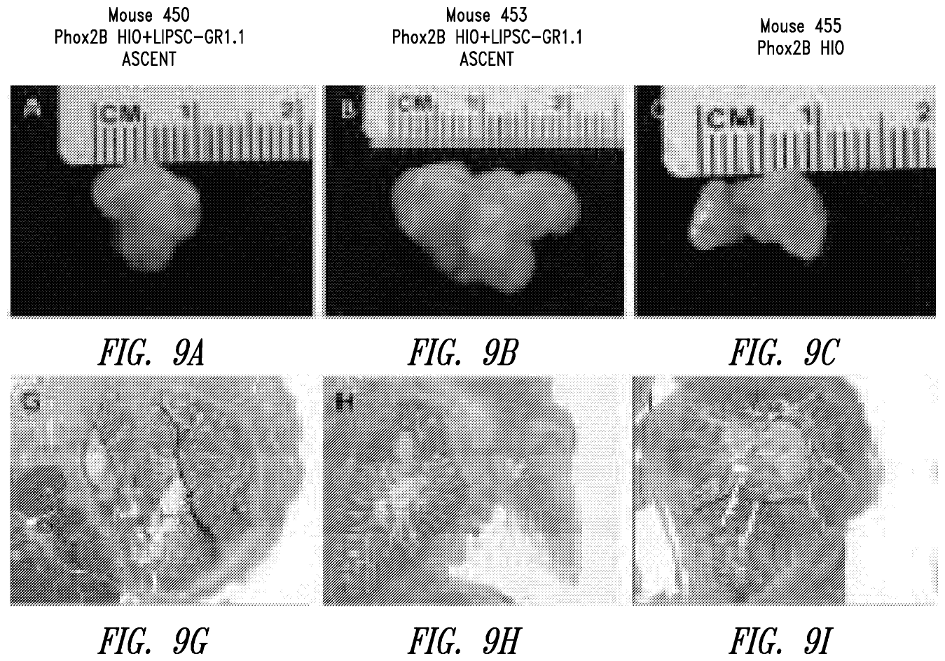

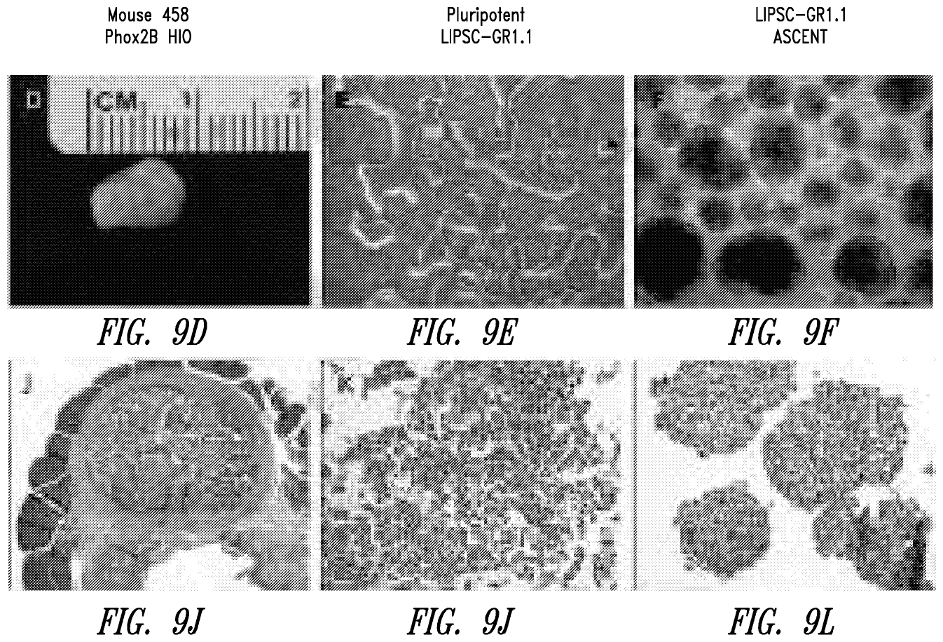

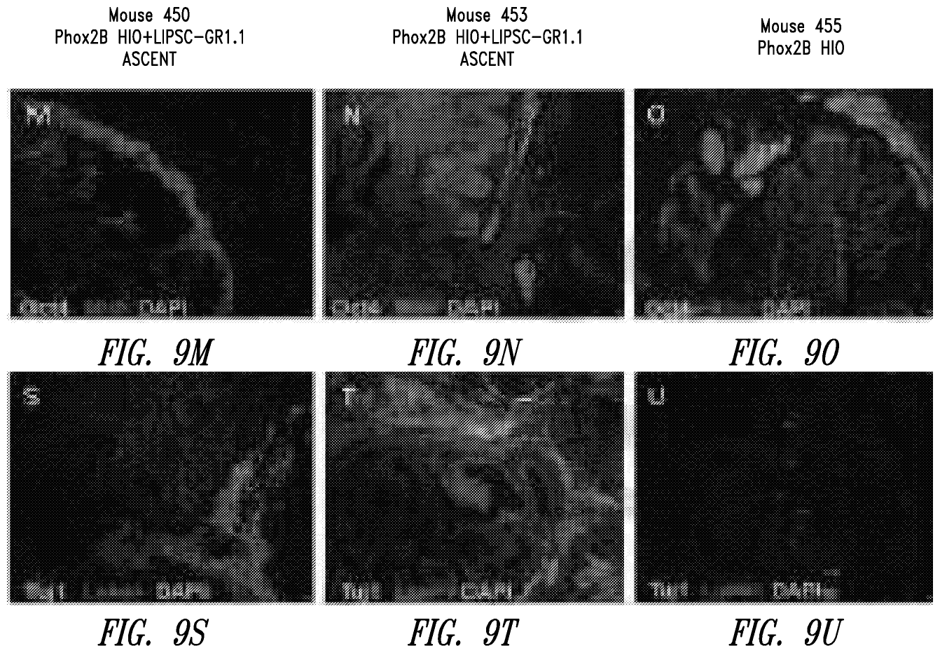

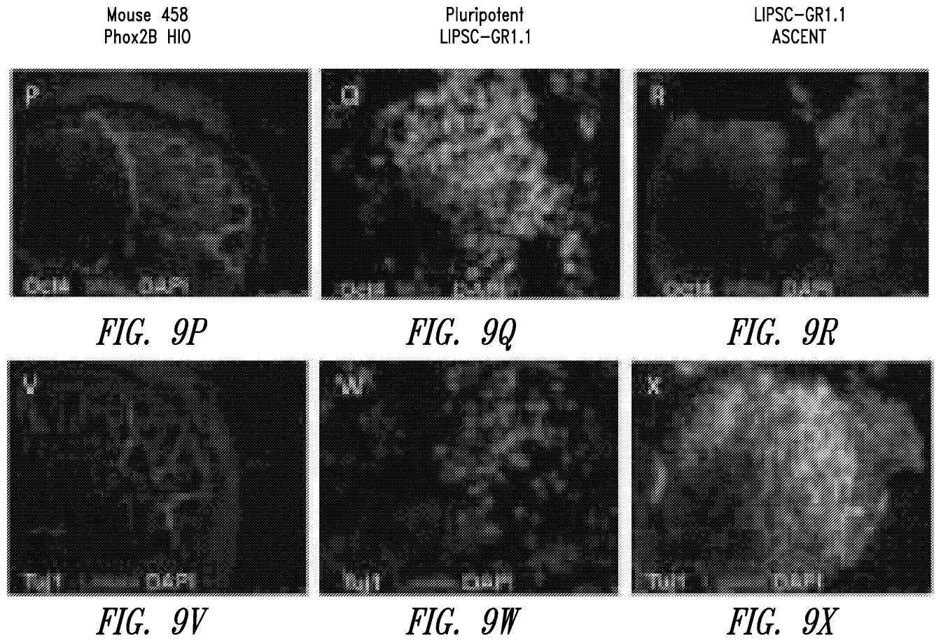

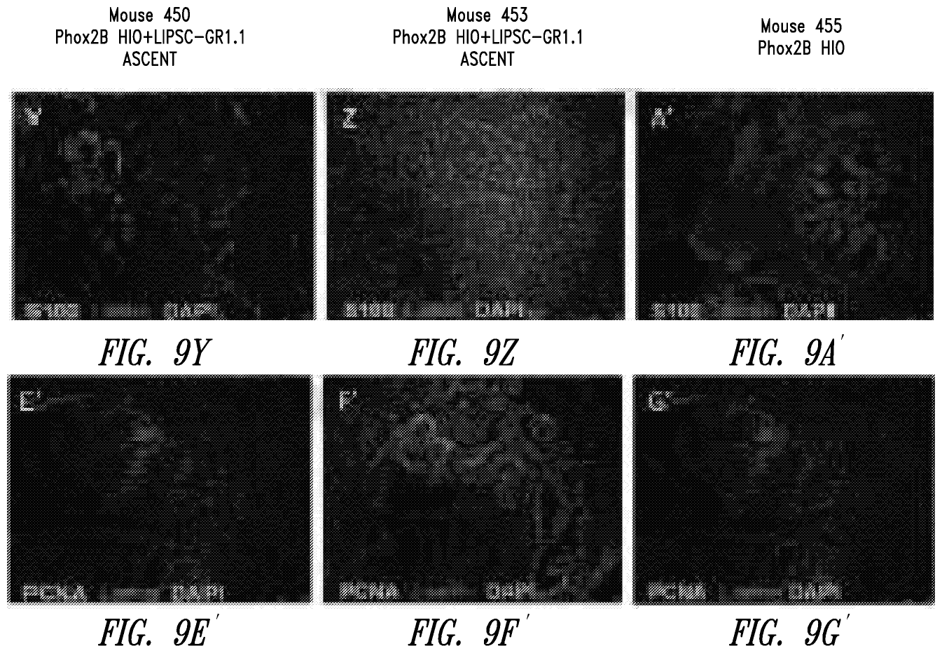

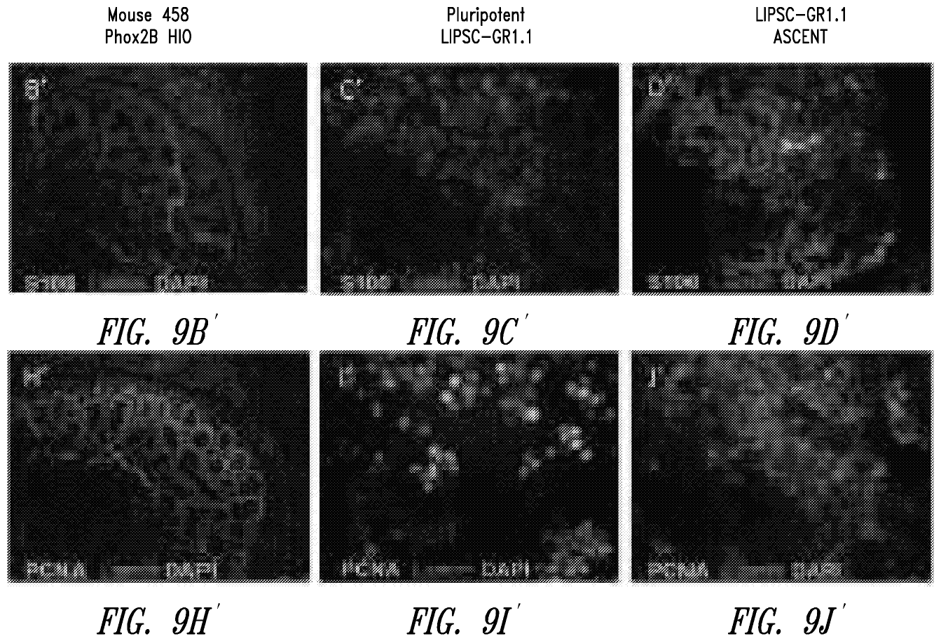

ENCC-HIO-TESI generated from PHOX2B-/- HIO and LiPSC-GR1.1 ENCC or PHOX2B-/- ENCC (control) demonstrate appropriately located differentiated components of the ENS generated from LiPSC-GR1.1 ENCC, but not the PHOX2B-/- mutant version. As described, PHOX2B-/- cells were differentiated into HIO26 and co-seeded with ENCC derived from LiPSC-GR1.1 cells onto a biodegradable polymer scaffold then implanted in NSG. The expectation is that the HIOs will differentiate into intestine lacking neurons, and ENCC will fully differentiate into ganglia and neurons throughout the tissue-engineered intestine, establishing components of the ENS in the aganglionic tissue. At 6-8 weeks, all explants lacked expression of pluripotency marker Oct4, and positively stained for human nuclear envelope marker lamin, neural markers Tuj1, glial marker s100b, and proliferation marker PCNA (FIG. 9). To confirm that the PHOX2B HIOs still differentiated into tissue-engineered intestine as does H9 (14), intestinal markers were analyzed after explantation. There was no difference in the intestinal histology including detection of epithelial cell marker E-cadherin, intestinal goblet cell marker mucin 2. Interstitial cells of Cajals detected with c-kit, enteroendocrine cells detected with Chromogranin A, and Paneth cells identified by lysozyme staining (data not shown).

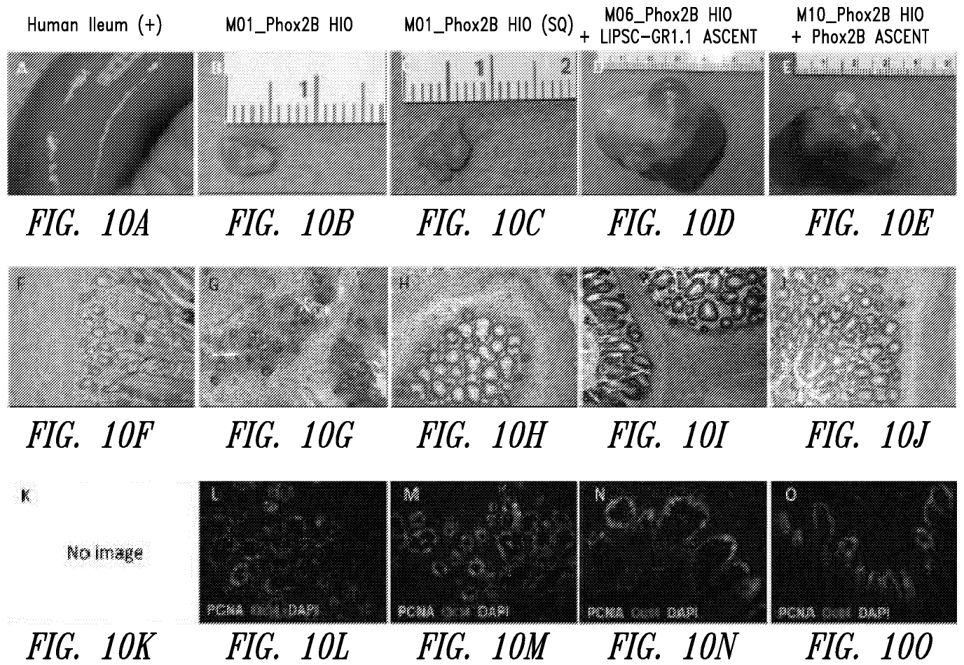

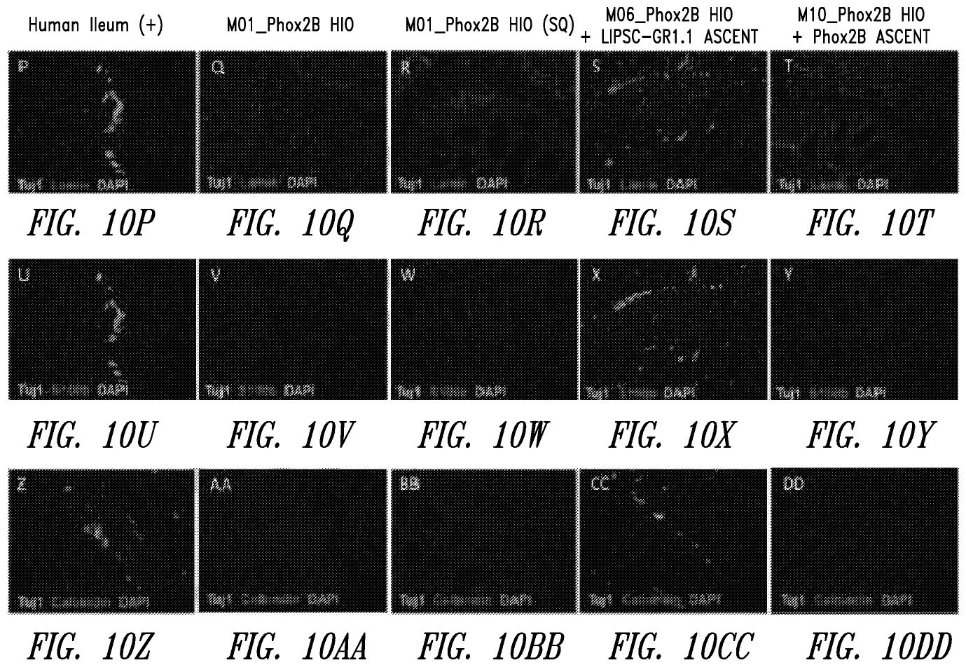

To determine the extent of differentiation of ENCC in vivo, further immunostaining analysis demonstrated the presence of neural and glial subtypes throughout the tissue, similar to that found in the H9 HIO-ENCC experiments previously reported. There is expression of neuronal calcium binding protein Calbindin, choline-acetyl transferase (ChAT)-positive excitatory neurons, and NOS-positive inhibitory neurons located in the appropriate anatomical sites when compared to human ileum when co-implanted with LiPSC-GR1.1 ENCC. These are notably absent if co-implanted with ENCC derived from PHOX2B-/- ASCENT cells. These data suggest that PHOX2B-/- HIO cannot generate any components of the ENS alone or when supplemented with ENCC derived from PHOX2B-/-. However, PHOX2B-/- HIO, when supplemented with LiPSC-GR1.1-derived ENCC appropriately generate all of the critical components of the ENS including neurons and glia (FIG. 10).

ENCC Restores Neuronal-Dependent Contractility and in ENCC-HIO-TESI. The nervous system control of the gastrointestinal tract is comprised of intrinsic neurons of the ENS and extrinsic sympathetic, parasympathetic, and sensory neurons of the CNS that regulate a host of processes, including peristalsis, migrating motor complexes, segmentation, mixing, digestion, absorption, secretion, excretion, and barrier defense (27.28.29). However. studies of externally denervated intestine have demonstrated that the ENS can autonomously regulate motility (30).

Gene analysis was completed for PHOX2B explants. Eight samples were compared by qPCR: Day 0 pluripotent LiPSC-GR1.1 cells, Day 15 ENCC from LiPSC-GR1. 1 cells, Day 0 pluripotent PHOX2B cells, Day 15 ENCC from PHOX2B cells, PHOX2B HIO only control from an omental implant (HIO_TESI), PHOX2B HIO only control from a subcutaneous explant (HIO_TESI_SQ), PHOX2B HIO+LiPSC-GR1.1 ENCC (HIO+LiPSC-GR1.1_TESI), and PHOX2B HIO+PHOX2B ENCC (HIO+PHOX2B_TESI). Eighteen different genes were analyzed, including pluripotency genes, neural plate border genes, and enteric neural crest genes, with the two housekeeping genes GAPDH and beta-actin (Housekeeping genes GAPDH and beta-actin: Pluripotency markers Oct4 and Nanog: intestinal stem cell marker SOX9; neural plate makers PAX3 and ZIC1: neural crest specifier makers SOX10, APA2, and SNAIL2: vagal neural crest markers HOXA2, HOXB3, HOXB5, ANlD HOXB7: ENCC progenitor marker PHOX2B: enteric neural crest cell markers p75, EDNRB. EDN3, RET, and GDNF). All curves were standardized to GAPDH and compared to Day 0 LiPSC-GR1.1.

Results of the qPCR analysis of the explants show decreased pluripotency marker Oct4 and Nanog for all samples except the two pluripotent samples, indicating that the differentiation process into ENCC is complete. Sox9 expression was increased in all the explants, indicating that intestinal development was occurring. There is increased expression of the neural plate borders in the PHOX2B HIO+PHOX2B ENCC compared to that of the LiPSC-GR1.1 ENCC, as well as neural crest specifier genes Sox10, APA1, and SNAIL2. The expression of the HOX genes vary, but the PHOX2B HIO+LiPSC-GR1.1 ENCC appears to have greater expression than the other explants. except for HOXB5, though the change in expression between the two does not appear to be significant. PHOX2B expression is increased in the PHOX2B HIO+PHOX2B ENCC (this could due to RNA that is unable to convert to protein, but this has not been confirmed). The enteric neural crest markers p75, EDNRB, EDR3 (ligand receptor for EDNRB), RET, and GDNF are all showing high expression in the PHOX2B HIO+LiPSC-GR1.1 ENCC in all except RET, which is consistent with past analysis.

To test for restoration of mechanical contraction in ENCC-HIO-TESI, implants were harvested and assessed with live-video imaging. HIO-TESI demonstrates spontaneous contraction even without supplementation with ENCC due to autonomic firing of ICCs. But this autonomic firing can be abolished in order to study the ENS contribution of ENCC by treatment of HIO-TESI with methylene blue, a known inhibitor of ICCs (31). After methylene blue treatment, a marked reduction in contractility is observed unless ENCC has been added to the HIO-TESI. Similar to HIO-TESI, ENCC-HIO-TESI also contracts after explant. However, treatment with methylene blue fails to completely block contractility in ENCC-HIO-TESI. Upon treating ENCC-HIO-TESI with tetrodotoxin (TTX), contractility was inhibited, suggesting that the enduring contraction and relaxation following methylene blue treatment is neuron dependent, from the implanted ENCC cells that have migrated and differentiated into an ENS network, and is independent of ICCs.

In non-GLP large animal studies, ENCC was injected into 6-8-week-old immunosuppressed Yucatan Miniature Swine, a large animal model that is the approximate size of the neonatal humans in whom the most severe cases of Hirschsprung disease are detected. The cell injection site described would be reasonable for both long segment or total intestinal aganglianosis versions of Hirschsprung disease. A dose of 6 million cells was split into two 3 million cell injections placed in the anterior and posterior taeniae coli with a 1 mL insulin syringe. These studies are examining mode of delivery, safety in delivery, as well as analyzing the migration capabilities of the cells in a model physiologically similar to that of a human infant.

Three Yucatan Miniature pigs were immunosuppressed with a combination treatment of Tacrolimus and mycophenolate mofetil. 6-8-week-old female Yucatan miniature swine were injected with day 15 ENCC in both the anterior and posterior aspects of the cecum with 2.5×10⁸ total cells.

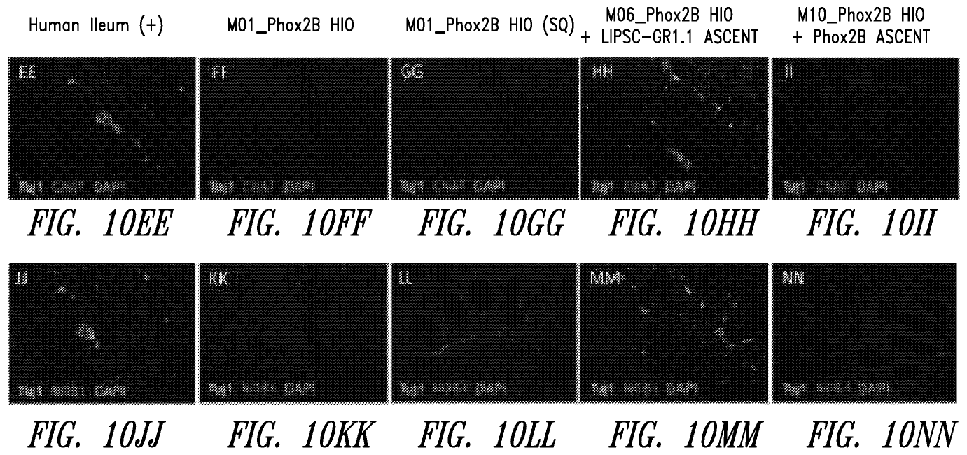

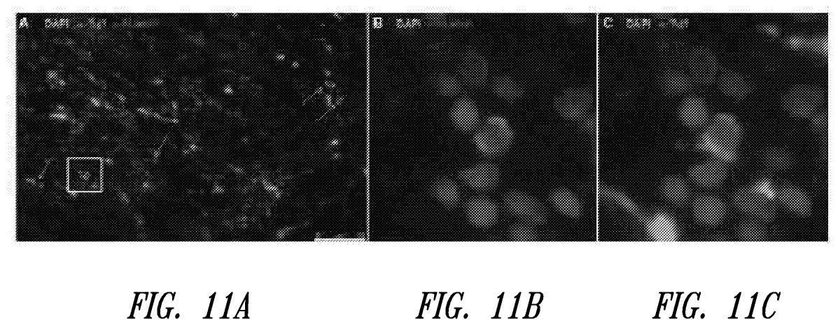

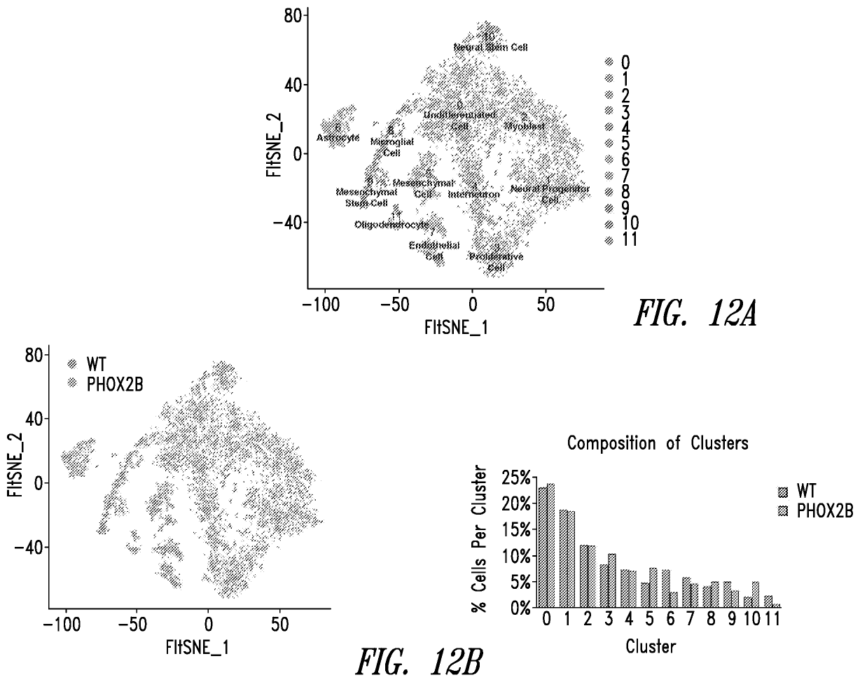

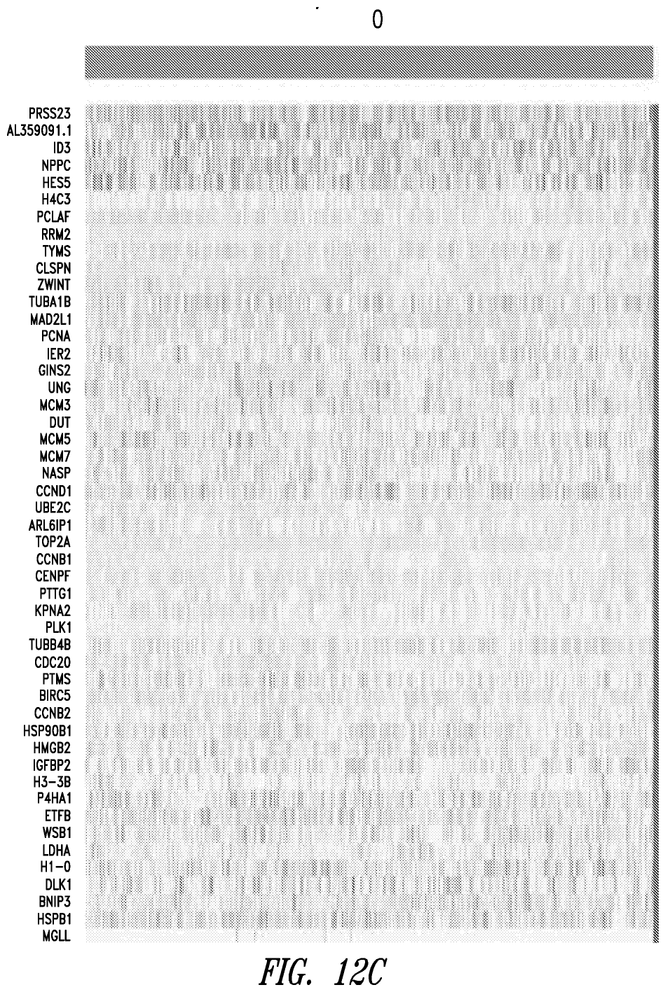

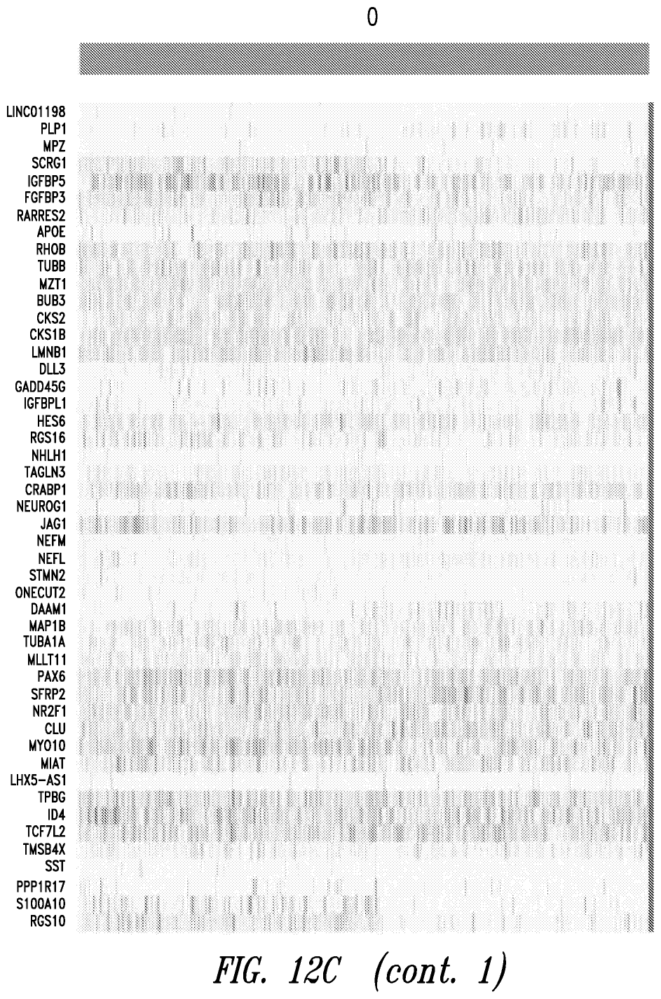

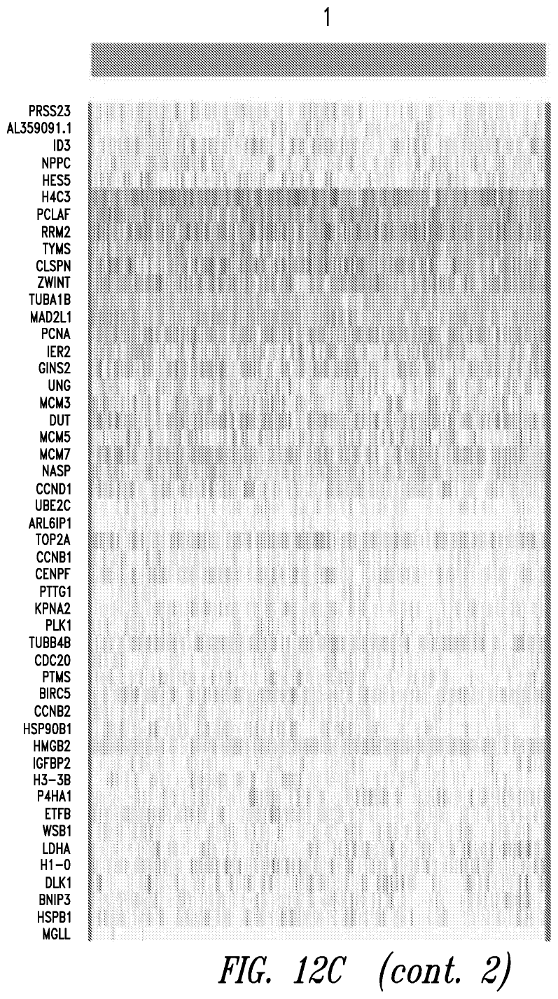

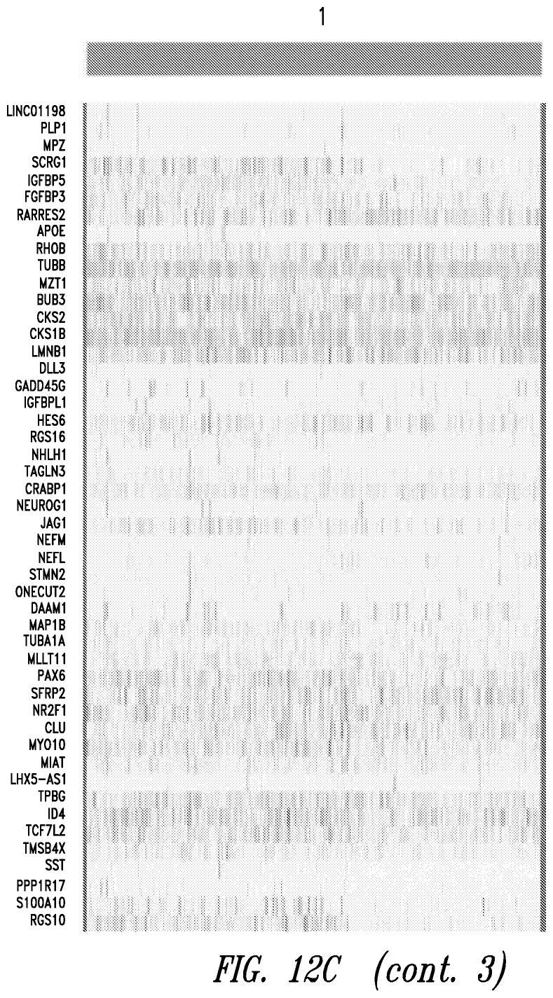

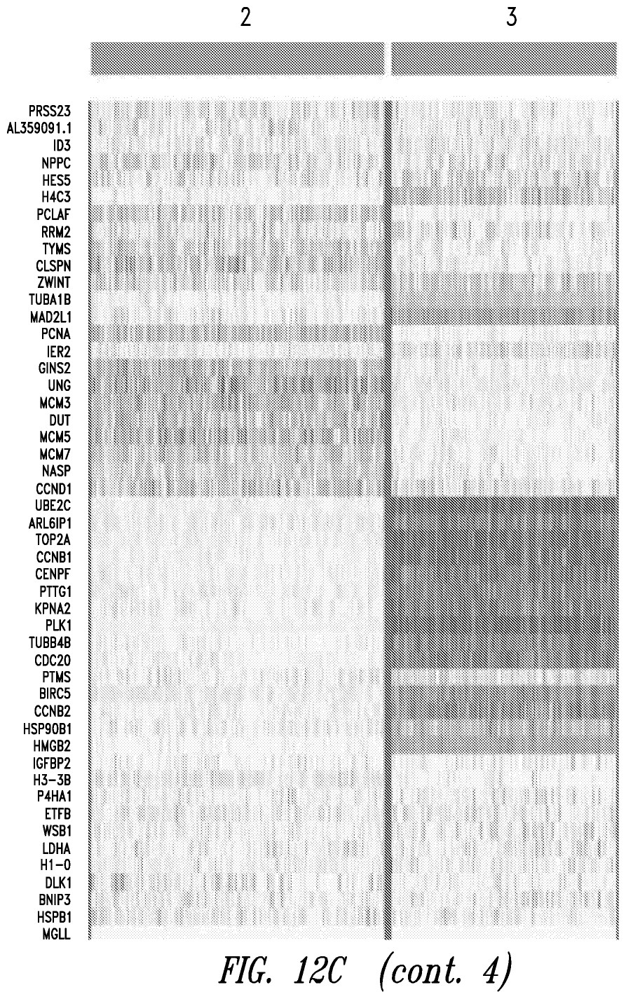

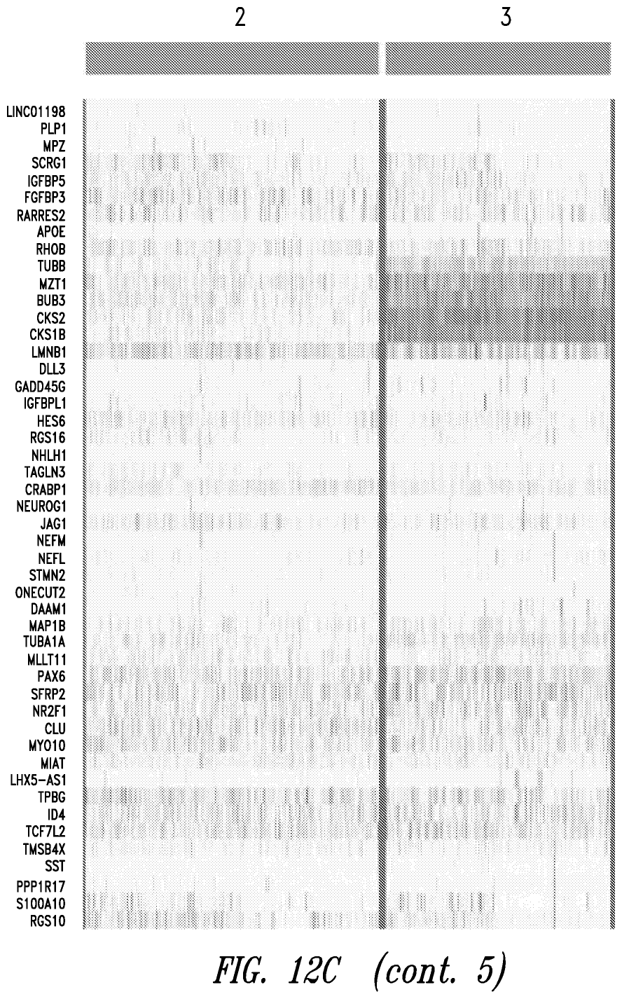

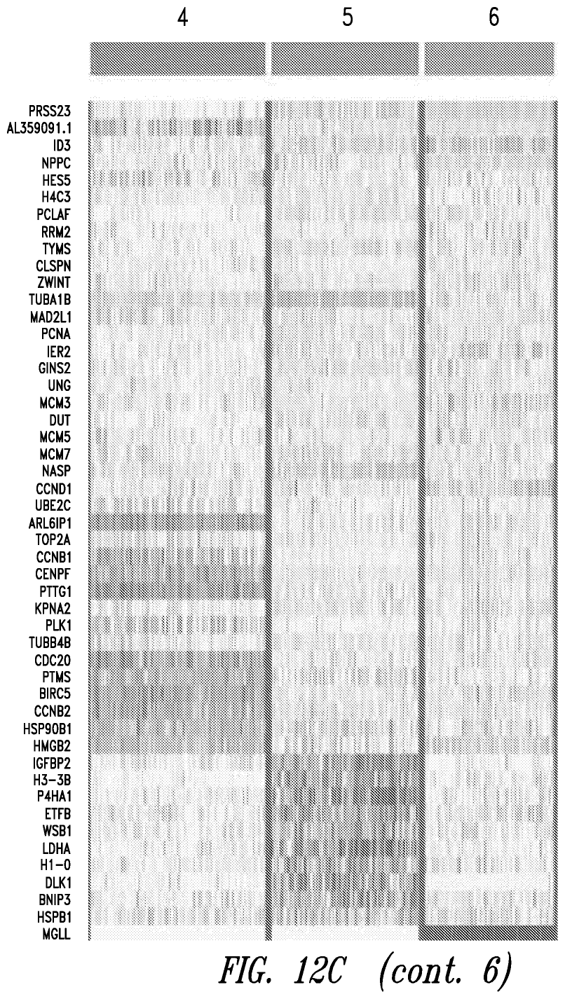

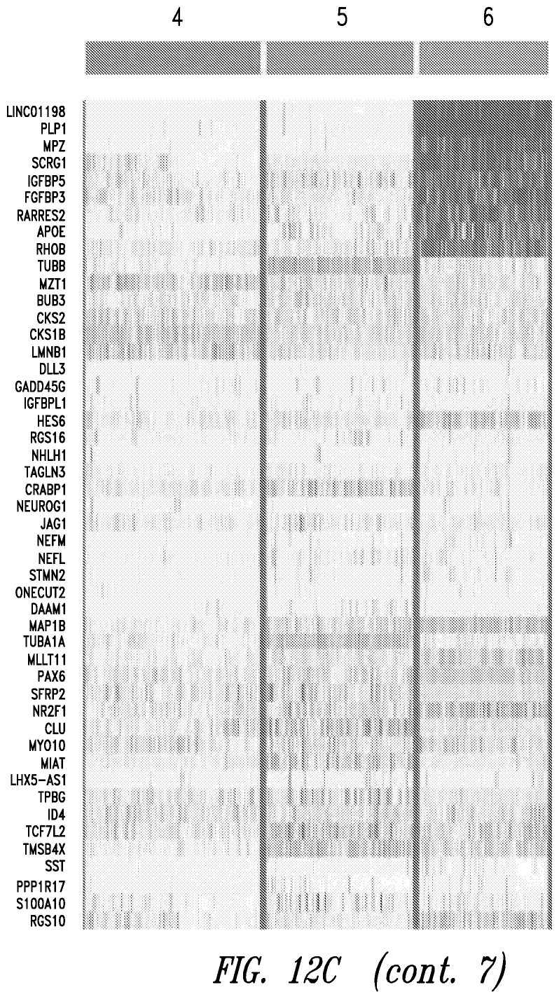

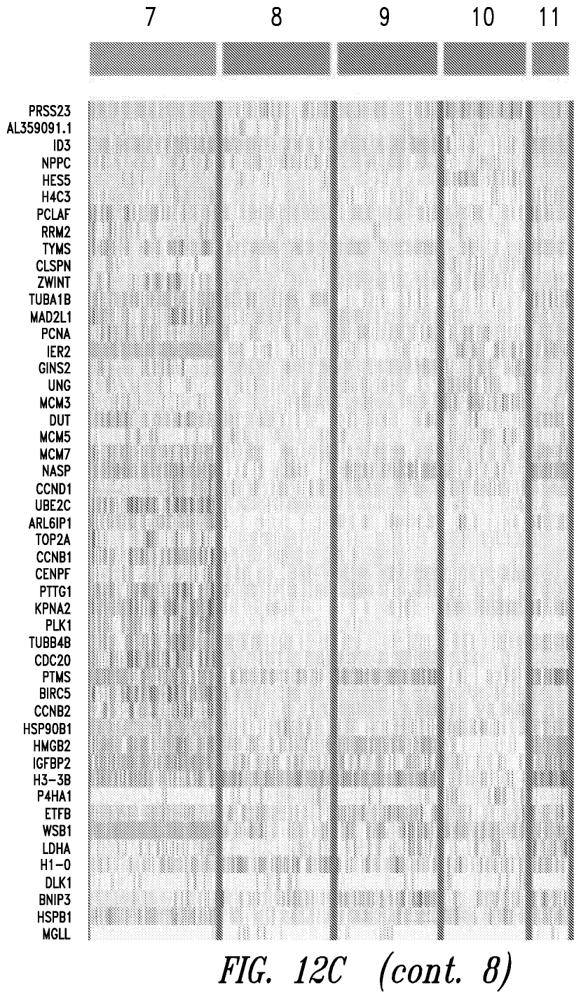

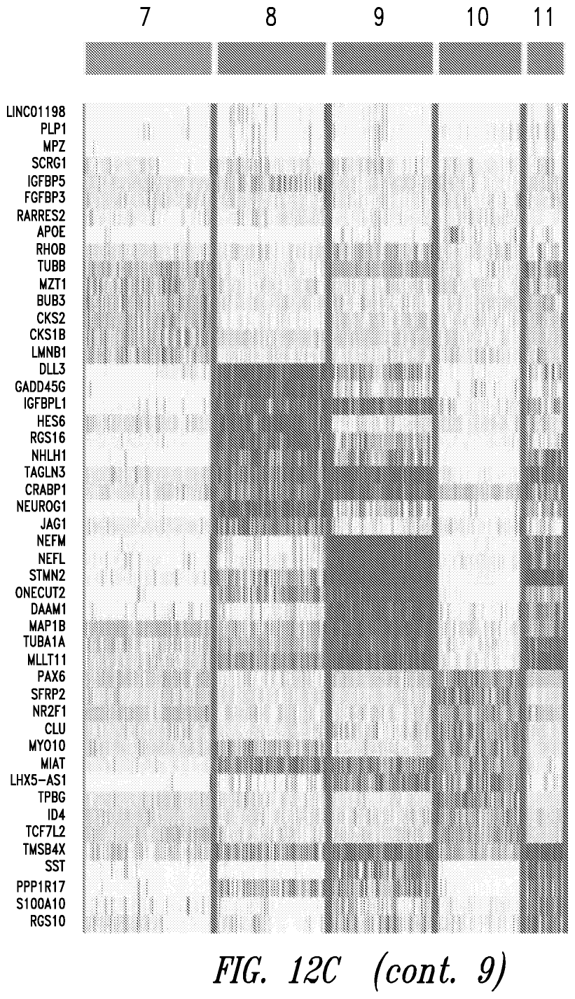

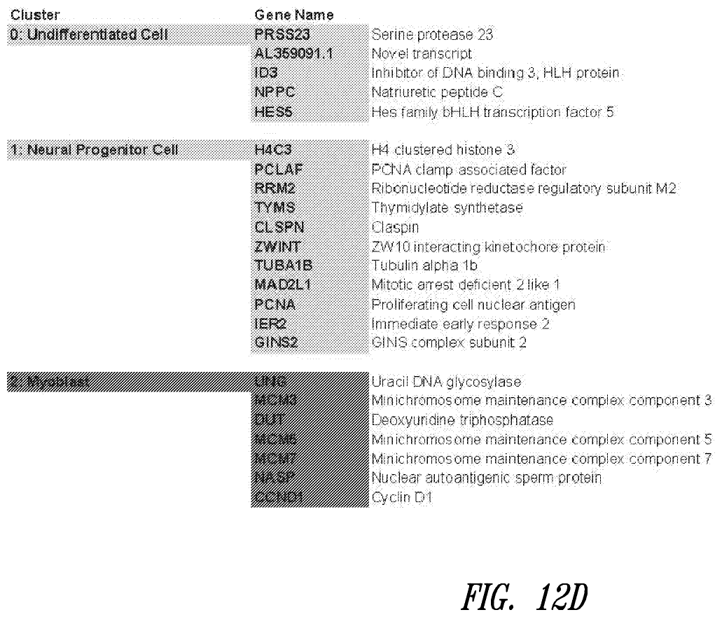

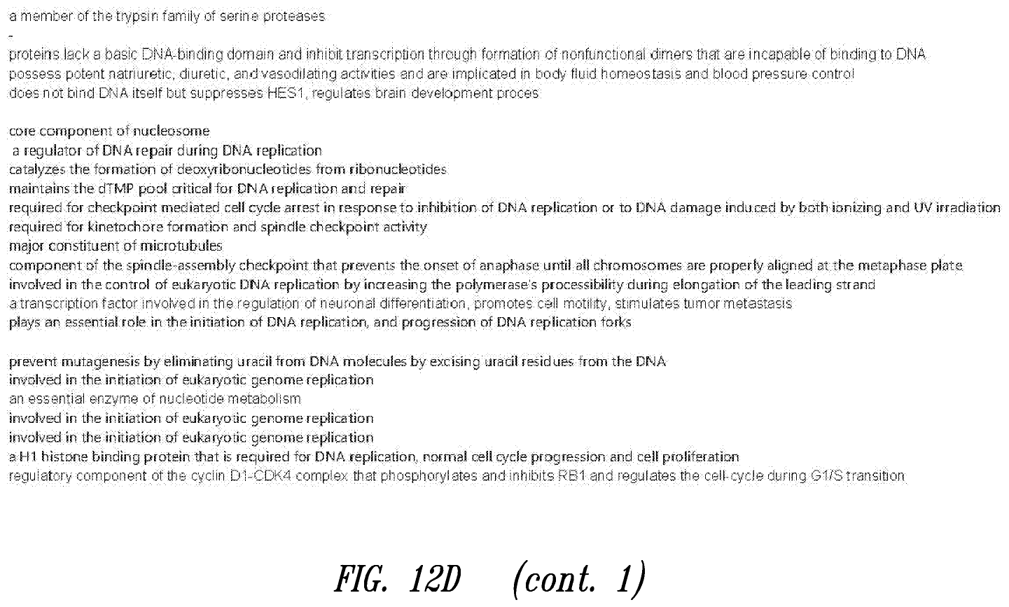

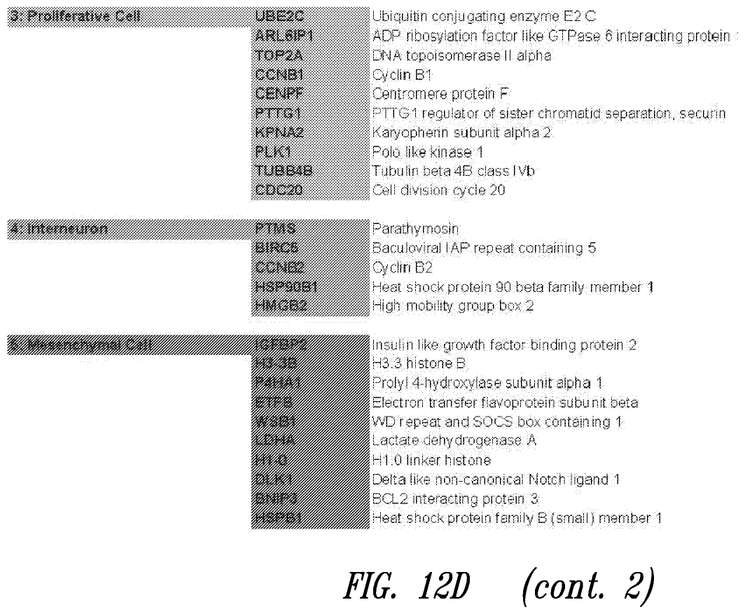

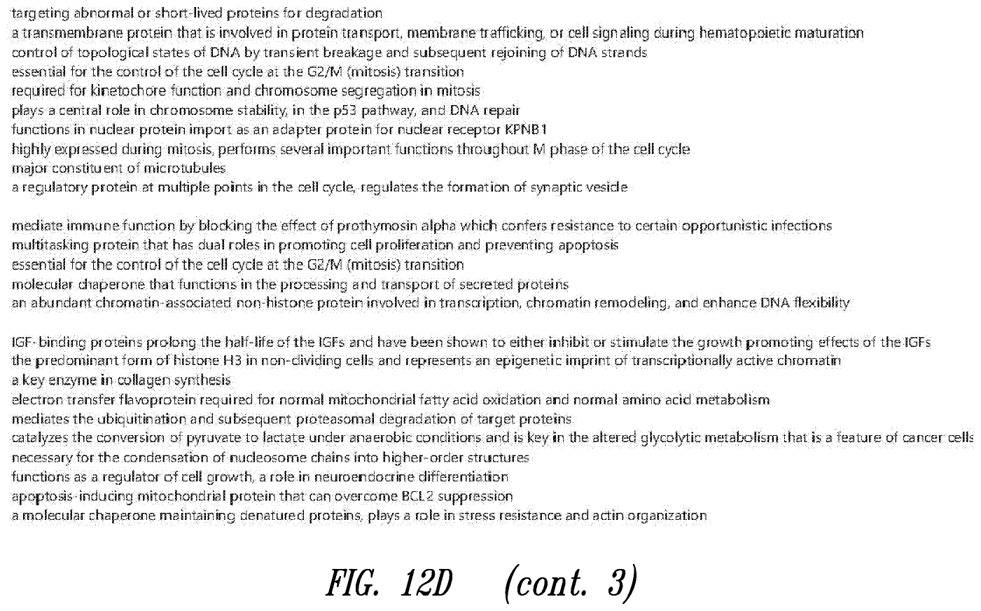

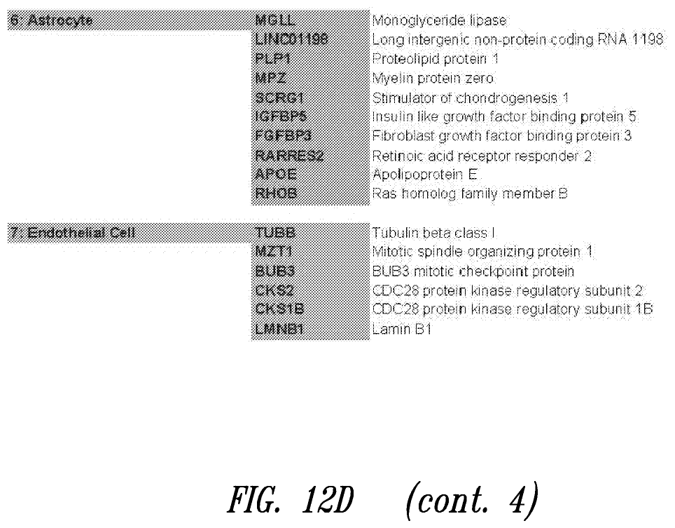

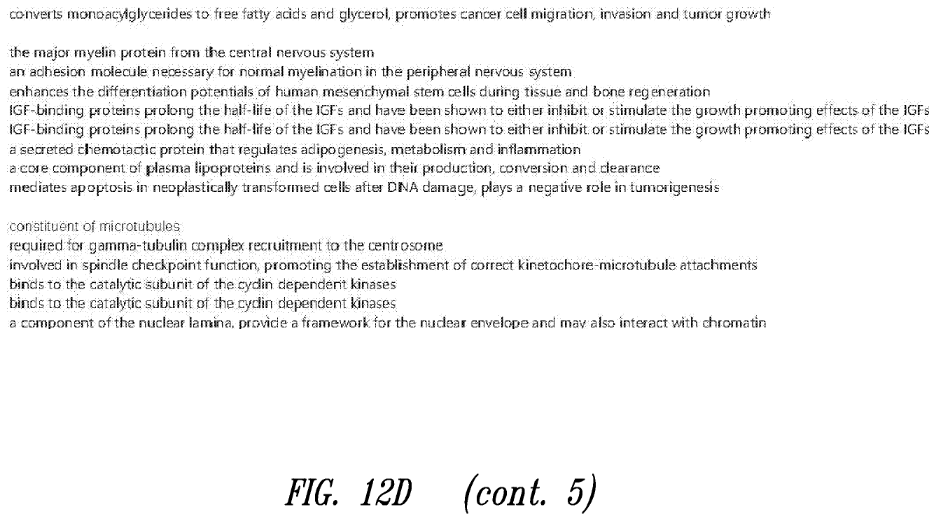

IF analysis of Pig 6267 (FIG. 11) was performed on sections of the intestine of 4 weeks post injection. Detection of human-specific marker lamin and neural marker Tuj1 identified human ENS cells derived from ENCC in the swine cecum at 4 weeks.

To determine the approximate number of human cells that survived implantation and immunosuppression in the swine, the tissue was processed and analyzed for the human ALU gene, an abundant small segment of DNA that contains a restriction enzyme site AluI, with over a million copies in the human genome.

To detect the ALU sequence in the Yucatan Miniature Swine tissue, a standard curve was made with human cells by extracting 100 ng of DNA from 4×10⁶ LiPSC-GR1. 1 to set up a standard curve for qPCR amplification of human-specific ALU (hALU) DNA sequence. The DNA was serially diluted 10-fold, creating a range of 1 ng/uL to 1/1,000,000 to make the qPCR threshold cycle (Cp) standard curve. The amount of ALU detected was then able to give an estimate of total number of human cells present based on the Cp value obtained comparing to the standard curve, e.g., 100 ng=4,000,000 cells, 10 ng=400,000 cells, 1 ng=40,000 cells, 0.1 ng=4,000 cells, 0.01 ng=400 cells, 0.001 ng=40 cells, 0.0001 ng=4 cells.

Human Alu was detected by qPCR in one pig tested in the areas near the injection site, the ileum, the ileocecal valve, and the colon. Human cells were also detected in 2 lymph nodes located next to the cecum, as well as some in the heart. In the second pig tested, human Alu was detected in two sites near the injection site

### Example 2

### Further Evaluation of Ipsc-Derived Stem Cells In Vitro nd In Vivo

As shown in FIG. 13, ENS components derived from ASCENT form a neural network within the intestinal wall of HIO-TESI, a model of human aganglionic intestine. These differentiated components of the ENS derived from ASCENT connect to the epithelium and develop in close proximity to c-kit+ interstitial cells of Cajal, as in wild type intestine. The area of the ENS components in HIO-TESI with ASCENT on histology is similar to the area of ENS components in fetal intestine.

After tissue clearing, on light sheet microscopy, ENS networks were visualized in coimplanted ASCENT-HIO-TESI whole explants, as compared to HIO-TESI controls. Tissue clearing allowed for optimal visualization of immunostaining for smooth muscle actin (SMA) and neurons (Tuj1). There are robust axonal projections traversing the myenteric layers within ASCENT-HIO-TESI, whereas HIO-TESI lacks Tuj1-positive neurons (41).

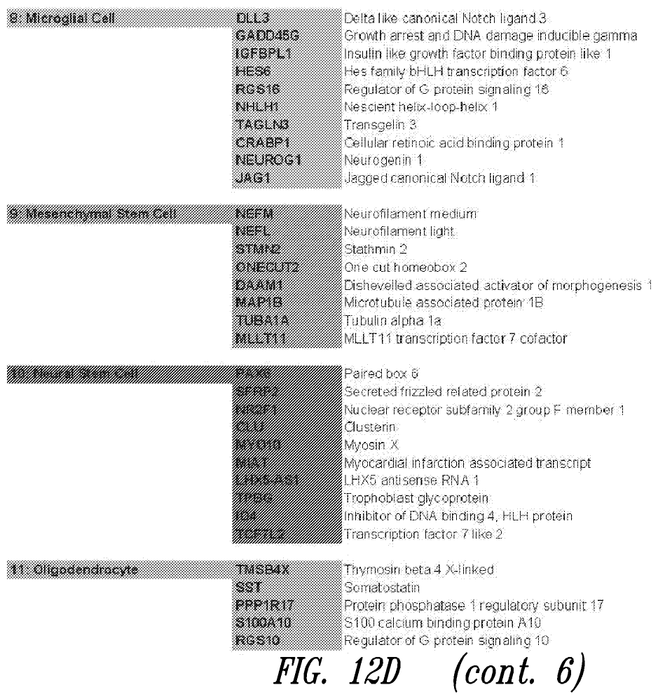

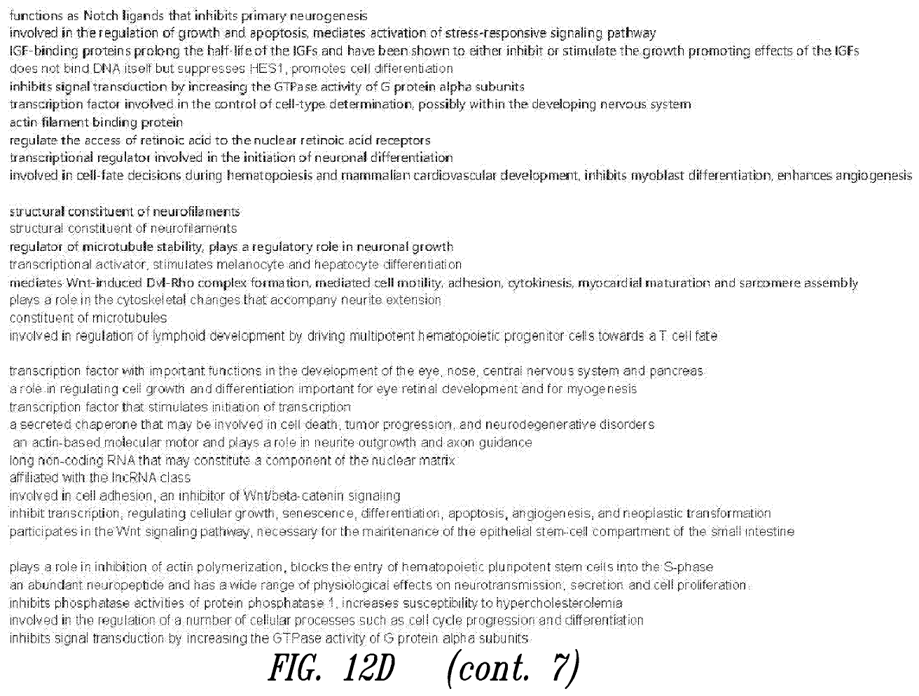

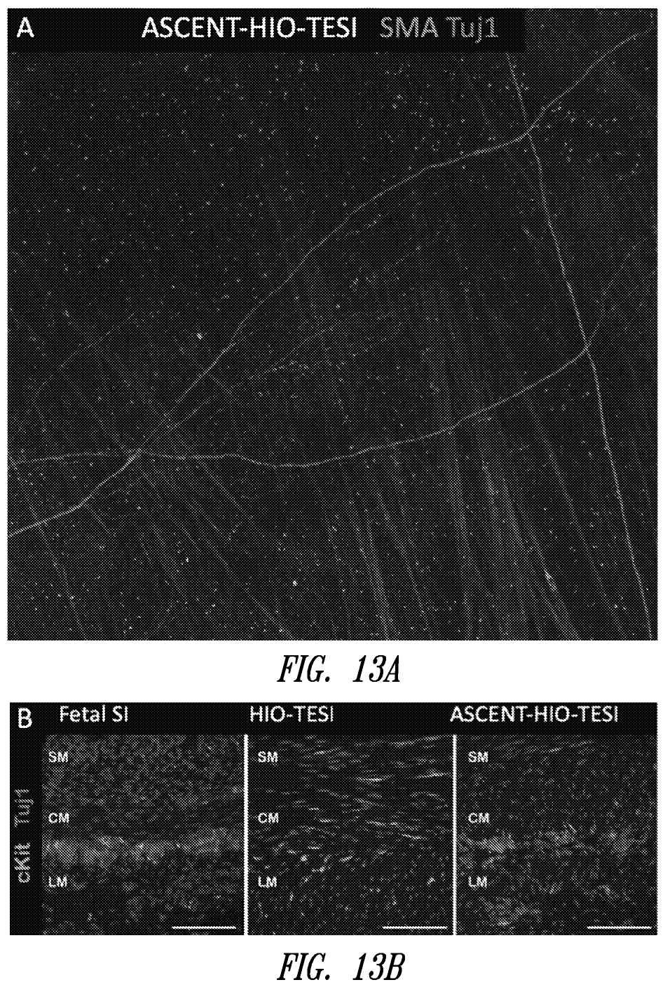

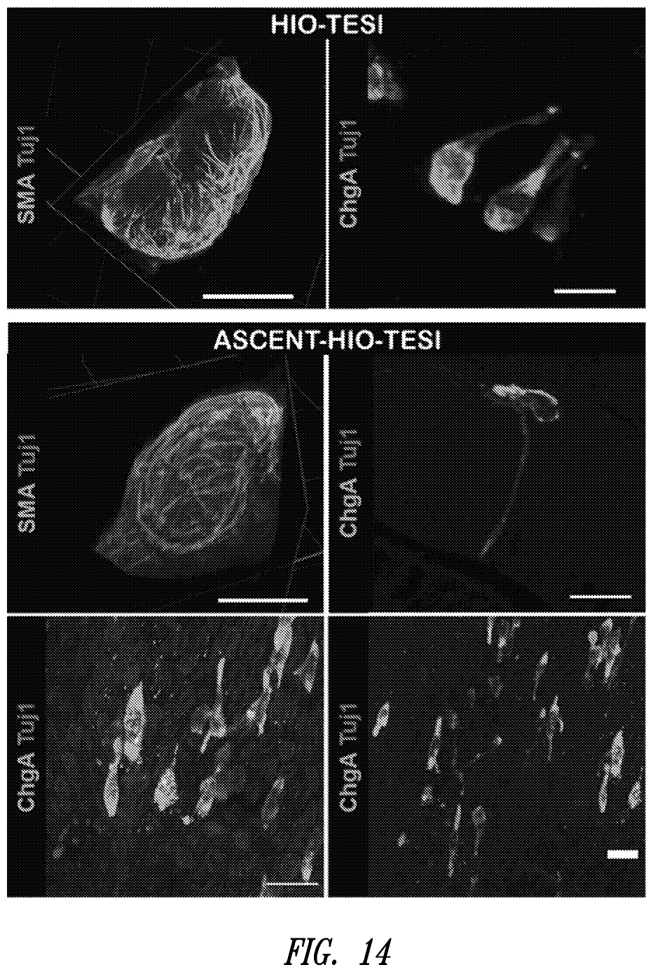

As shown in FIG. 14, Intraluminal sensory signaling from enteroendocrine cells is critical in regulating numerous processes in gastrointestinal tract function, such as mixing and propulsive activity, release of digestive enzymes, activation of nutrient transporters, fluid transport, and immune responses (42). The propagation of these signals occurs through connections between EEC and underlying neurons at the ECC neuropod (43). Evaluation by 3D confocal microscopy of coimplanted ASCENT-HIO-TESI demonstrated synapses between enteric neurons and EEC (44). Tuj1-positive neuronal axons projected to EEC cell bodies suggesting establishment of neuroepithelial synaptic connections (FIG. 14).

An interaction between ENS components and the intestinal epithelium was also determined when reviewing RNA-seq of ex-vivo coimplantation ASCENT-HIO-TESI (41), which identified markers of gliogenesis and neurotransmission. For mature epithelial cell types, there were no significant differences in absorptive enterocyte markers in in vivo ASCENT-HIO-TESI but there was a significant upregulation of Paneth cell gene LYZ and significant downregulation of enteroendocrine cell genes GCG and NTS. There were also significant decreases in several goblet cell genes.

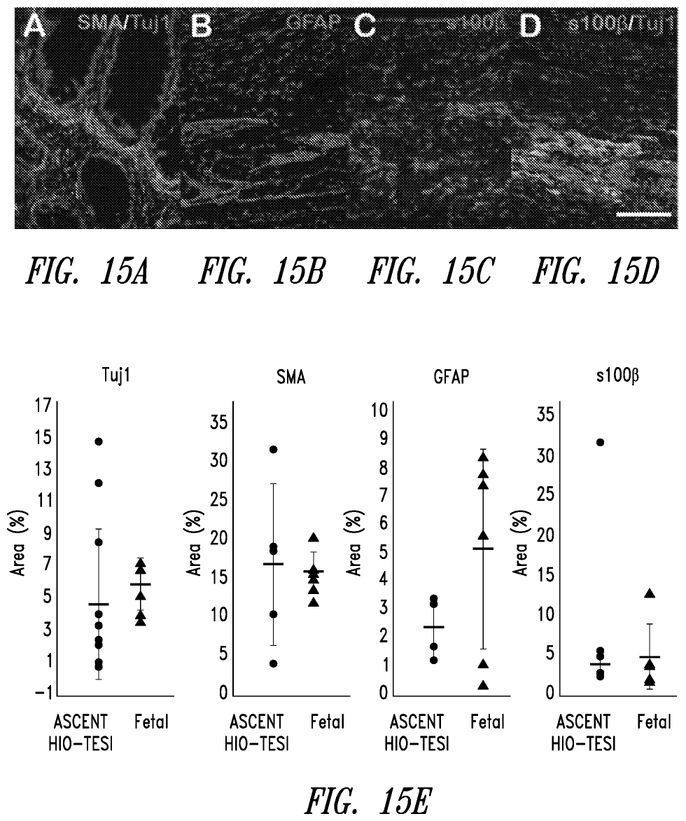

As shown in FIG. 15, supplementation of HIO-TESI with ASCENT results in key components of the ENS that localize appropriately and contribute neuron-dependent contractile function to these constructs. The areas of ENS components are similar to fetal intestine (44).

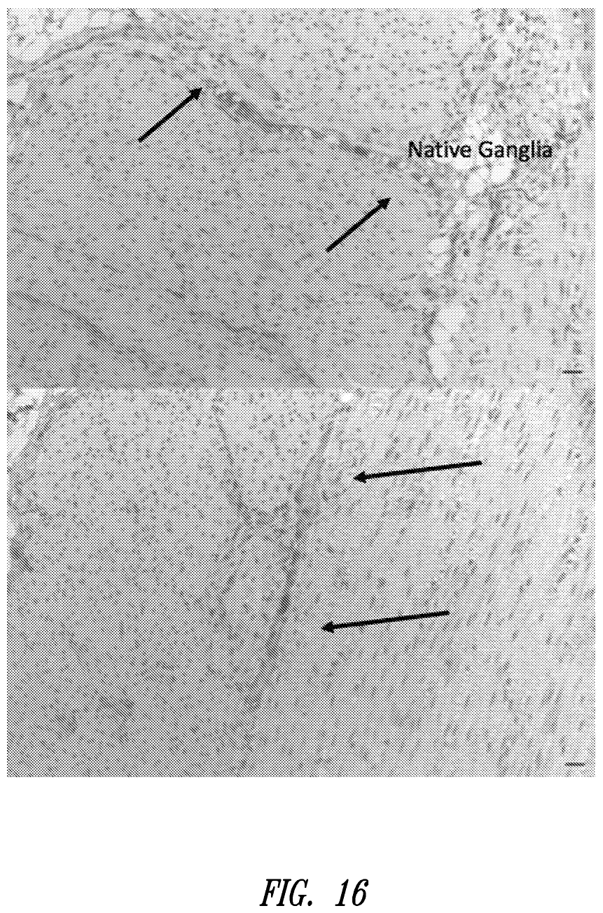

FIG. 16 shows Ku-80 staining (human marker, brown) and identifies neural structures traversing the intestinal wall muscle layers in pig 6577 connecting to the pig's native Meissner's plexus. It is known that progenitor cells survive and differentiate well when they have access to a supportive microenvironment, or niche, and in the swine model we have not created a stem cell niche through injury or absence, but rather supplemented a healthy wildtype ENS with ASCENT, which, remarkably, do nevertheless localize in proximity to other ENS components. As shown in FIG. 16. the neural network formed after ASCENT injection in the HIO-TESI model is more robust after 10-12 weeks than the results in a xenogeneic model after 4-6 weeks, which is so far the maximum amount of time we have been able to maintain these very young swine for humane endpoints.

As shown in FIG. 17, neuronal-dependent contractility and relaxation can be quantified in ASCENT-HIO-TESI. We quantified the mechanical activity of ASCENT-HIO-TESI at two different doses of ASCENT (E6, E7) that have had three months of development. To quantify mechanical contraction of ASCENT-HIO-TESI, we developed a standard operating protocol to perform automated video analysis of the implants after harvest and treatment with methylene blue to abolish contraction that was caused by the presence of ICC. This automated video analysis allows point masses to be chosen on the sample, after calibration. Movement in the x and y axis is then captured by image recognition with standardized parameters. The data are compared to a stable control point on the dish surrounding the sample to remove motion artifact. The Tracker program (version 6.0.4) is a video analysis and modeling tool built on the Open Source Physics (OSP) Java framework.

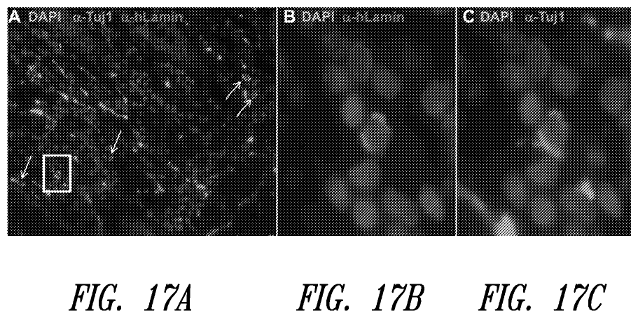

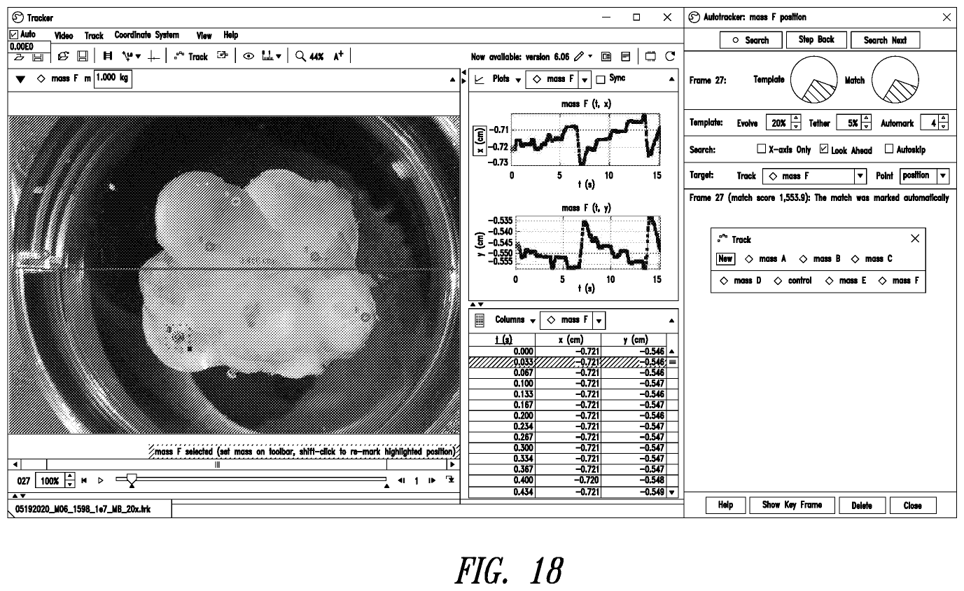

FIG. 18 shows an example of an ASCENT-HIO-TESI sample after treatment with methylene blue during video analysis with Tracker. The Tracker program is a video analysis and modeling tool built on the Open Source Physics (OSP) Java framework. A blue calibration stick is visible. Points of mass, labeled mass a- mass f, are identified at random at points of high contrast, with an example of the template and required match for mass F seen on the right. This high contrast area becomes the template for comparison throughout the video, which is analyzed at 20× speed in order to work with a computable number of frames. The program then searches each frame of the video for images that match the template, and assigns a match score. Match scores above 3 are accepted without used input. Any match below 3 can be reviewed by the user or skipped. The movement of this mass point is plotted on a coordinate plane, assigning X and Y values to the calibrated distance moved over time. The template area of mass point F (black) can be seen in the Autotracker window, along with the corresponding coordinates and plots. A control point on the side of the dish (red arrow) measures any movement of the dish or vibrations that caused the dish to move, and these measurements are matched and removed from the data for every time point prior to graphing.

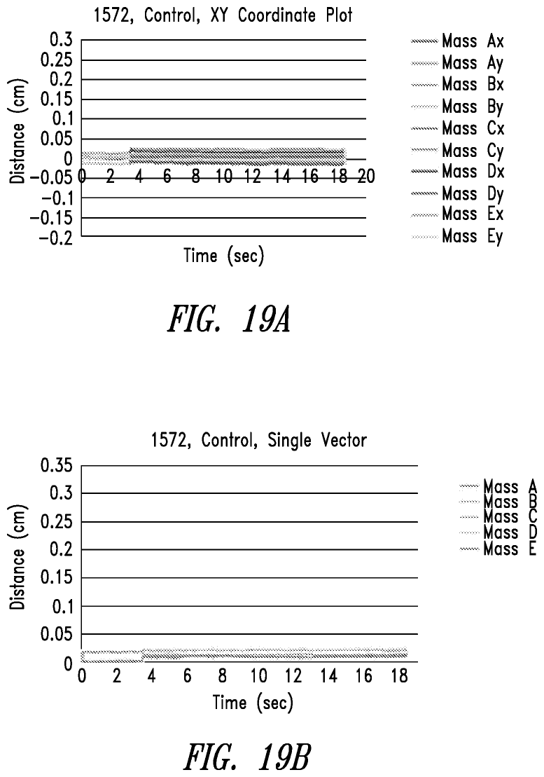

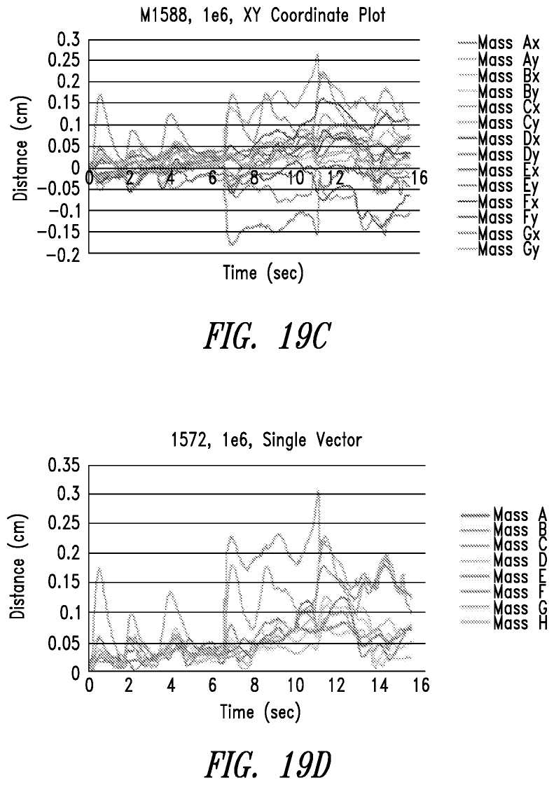

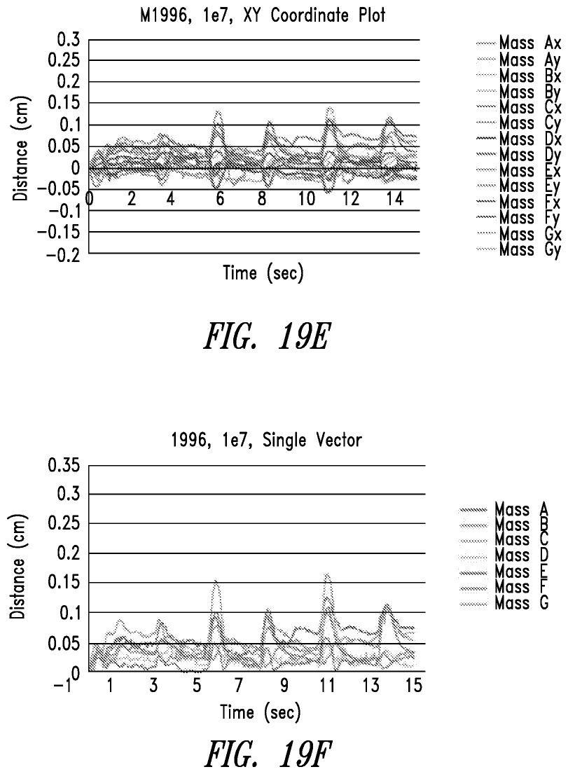

After correcting for motion unrelated to the contractility of the sample, the x and y coordinate plot was generated for each sample, allowing further calculation of a representative single vector, according to the Pythagorean theorem. The single vector calculation simplifies the data for the viewer as is seen in FIG. 19. In both cases, the control does not exhibit periodic movement which we interpret as contraction and relaxation, whereas in both E6 and E7 doses movements at intervals that we interpret as contraction are definitely measured.

As shown in FIG. 19, data points collected by Tracker were exported to and graphed in Microsoft Excel to quantify the amplitude of contractions from 3-month HIO-TESI with and without ASCENT. Here are examples of data generated for three different conditions with two different vector calculations, A, C, E) Data from Tracker was plotted as XY Coordinate scatterplots, with distance in centimeters on the y-axis and time in seconds of the 20× videos on the x-axis, B, D, F) Single vectors were generated from the XY data for each sample, again matching the XY data seen on the left for Control HIO-TESI (A,B) and

ASCENT dose E6 (C,D) or E7 (E,F).

Response 1.21: Functional readouts and the impact on dose and redosing strategies

We then compared the amplitude distribution of the various conditions, with additional analysis of isolated mouse intestine that had been resected, flushed, and treated with methylene blue according to the same protocol. Both doses of COH ASCENT injected into HIO-TESI demonstrated greater contractility than either the controls or native mouse intestine (FIG. 11). We interpret these data to mean that both E6 and E7 doses in the HIO-TESI model impart improved contractility, and we will determine a final dose after the IND-enabling studies proposed in this application.

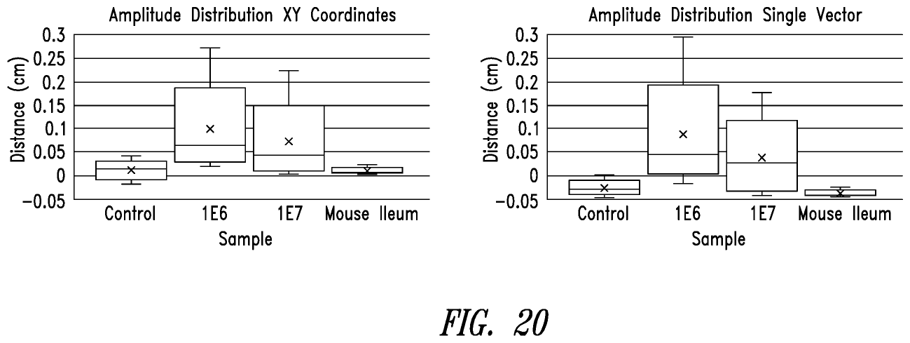

FIG. 20 shows amplitude distribution for four conditions calculated from XY coordinate and Single Vector Data. The black line represents the median, the box represents the interquartile range, and the whiskers represent the maximum and minimum values of the data set. The clinical protocol will minimize the time ASCENT is thawed prior to injection by only beginning preparation of the syringe when the intestine is ready in the surgical field to receive the injection. In the pig models the eight injections from one vial are performed in well under ten minutes. The Thaw Star CFT2 take 2-3 minutes to thaw the vial, and ASCENT is immediately taken up into the syringe and diluted to the appropriate concentration according to the SOP. The thaw and syringe loading protocol takes approximately 10 minutes, and the injection of all eight doses from the vial into the intestine can be completed in approximately 5-10 minutes. Therefore, although we are currently testing strategies to increase the measured viability at later timepoints, we do not envision ever injecting cells at these later timepoints. In patients who receive escalating doses, additional vials will be thawed one at a time to minimize holding conditions.

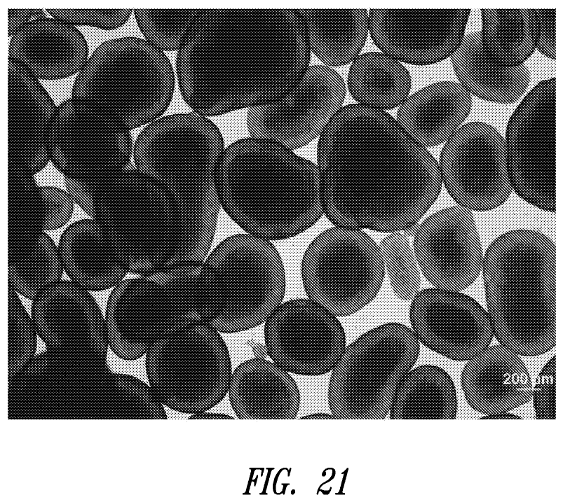

FIG. 21 shows the final ASCENT product captured after differentiation at COH with scale bar=200 um, showing that ASCENT neurospheres are very large cellular aggregates.

### Example 3

### Clinical Trial Design and Evaluation

### Study Title

A phase 1/2a clinical trial to assess the delivery and safety of Advanced Stem Cell Enteric Neuropathy Therapy (ASCENT) generated from human induced pluripotent stem cells for patients with long segment or total aganglionosis variants of Hirschsprung Disease.

### Study Objectives

Primary objectives: To assess the feasibility of the delivery and safety of ASCENT in patients with Hirschsprung Disease (HD) with long segment aganglionosis (LSA) or total aganglionosis (TA).

Secondary objectives: The secondary and exploratory endpoints will assess:
The motility of the treated intestine as determined by endoscopic solid state or water-perfused manometric systems with calculation of the motility index (MI) and pharmacologic provocation (octreotide or bisacodyl)
High amplitude propagated contractions (HAPC, colon) or and migating motor complex (MMC, small intestine) quantification.
Episodes of Hirschsprung's associated enterocolitis.
Resting internal anal sphincter pressure (IASP).
Recto-anal inhibitory reflex (RAIR).
The presence of ASCENT cells, neural cells, ganglia, or any aberrant tissue growth in biopsies of treated intestine post administration of ASCENT.

### Study Rationale

Children with Hirschsprung Disease (HD) have a congenital enteric neuropathy that is caused by the failure of complete migration of the enteric nervous system (ENS) into the distal intestine during in utero development, resulting in aganglionic (no ENS) and therefore non-functional intestine of varying lengths. Most children with HD have an enteric neuropathy up to the rectosigmoid junction, in which the aganglionic intestine can be resected with moderately good results. However, in patients with total aganglionosis (TA) where there are no ganglia in the entire small and large intestine, the disease is life-threatening with frequent mortality. Many parents opt for withdrawal of care. Intestinal transplantation is formally an option for some patients, but because of low availability, and high morbidity and mortality associated with the transplant, intestinal transplantation is often avoided. For patients with long segment aganglionosis (LSA) no ganglia are identified until a site proximal to the large intestine. In LSA, the disease is less severe, however LSA is not currently well-managed (See Value Proposition and Risk/Benefit Profile above.) Preclinical animal models to date have shown that ASCENT has the potential to survive in aganglionic tissue and induce increased contractility. Pivotal IND-enabling studies will be conducted to further establish the potential for safety and efficacy of the final ASCENT product. The ultimate goal of ASCENT therapy is to restore components of the ENS to the diseased intestine and to establish sufficient contractility so that a patient can have a meaningful increase in available functional intestine. This first-in-human clinical study is designed to test, as the primary endpoint, safety of administration of ASCENT and of the ASCENT cells in patients who have had leveling surgery for their disease. In leveling surgery, after serial biopsies to determine the presence or absence of ganglia, the intestine bearing developed ENS components is connected to the abdominal wall through a stoma. The intestine that does not have an ENS is maintained as a defunctionalized segment of intestine connected to the abdominal wall through a mucus fistula (MF) (see FIG. 14 below). In this study, ASCENT will be injected in the wall of the detached, defunctionalized segment of the intestine. Doses will escalate for subsequent patients. Secondary and exploratory endpoints will measure changes in histology and functional status of the treated intestine. If motility measurements identified an improvement in the motility index and biopsy identified the presence of ENS components, then reconnection to the patient's natively ganglionated intestine could be entertained. This would be considered in conjunction with the DSMB, FDA and parents of the subject.

### Study Population

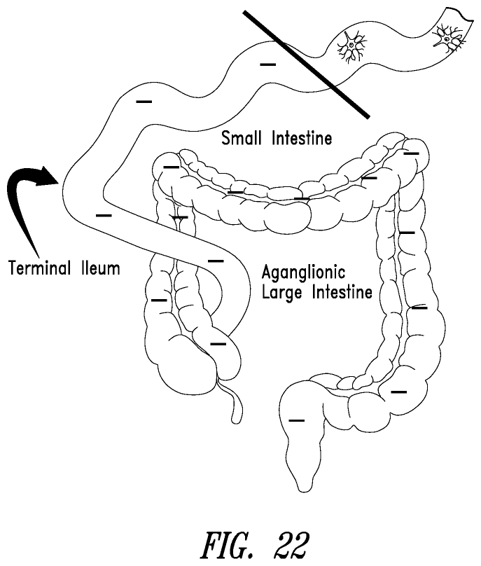

The study population will be patients with Hirschsprung Disease (HD) with biopsy confirmed long segment aganglionosis (LSA) or total aganglionosis (TA) who have had leveling surgery. As shown in FIG. 22, all children with LSA or TA will have multiple biopsies of the intestine prior to dividing the intestine where there are abundant components of the ENS above the division (shown as blue ganglia in this figure) and an absence below the division (shown as minus signs).

Stoma formation (opening the intestine to the abdominal wall for egress) occurs in the operating room for children with HD after leveling biopsies, which are serial samples of multiple regions of the intestine. A pathologist diagnoses the presence or absence of ganglia, a major component of the ENS, by frozen sections examined under the microscope. Samples are taken higher and higher on the intestine, traveling from the distal end of the intestine until ganglia are identified, which is the level of the disease. If LSA or TA is diagnosed by the absence of ganglia in sections (Figure X), the intestine bearing ganglion cells is diverted to the abdominal wall as a stoma and the intestine without an ENS is diverted to the abdominal wall as a similar opening, termed a mucus fistula (MF) (Figure Y). The stoma empties from the mouth downward to allow the intestine that does not have an enteric neuropathy to function. In patients with TA, this will be a very short segment. The MF allows egress from the diseased segment that will still produce mucus. The diseased segment cannot empty well from below because it does not have an ENS.

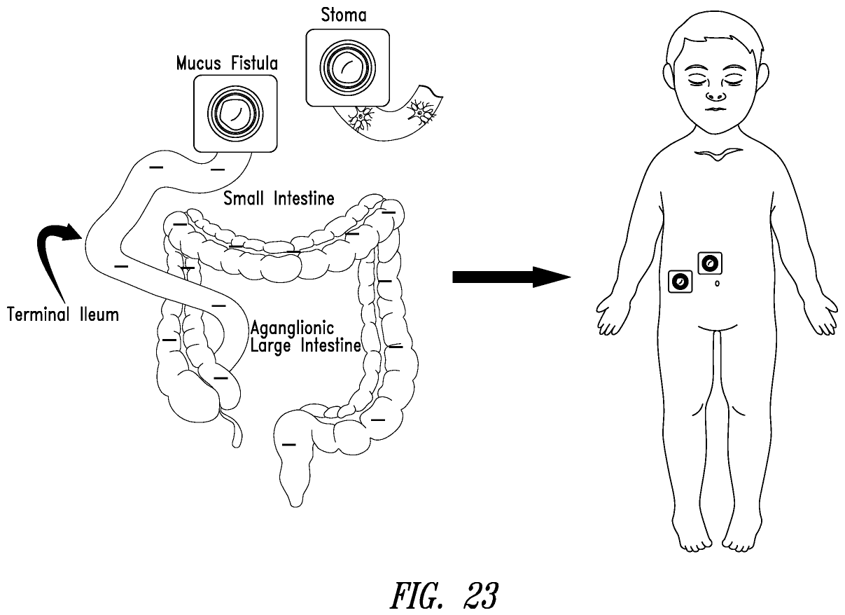

FIG. 23 shows the positioning of the stoma and mucus fistula (MF) on the child's abdomen after leveling biopsies are completed.

### Main Inclusion Exclusion Criteria

### Inclusion Criteria:

LSA or TA proven by multiple biopsies having received leveling surgery with the functional intestine connected to the abdominal wall through a stoma and the defunctionalized intestine connected to the abdominal wall through a mucus fistula.
No previous corrective intestinal surgery except for leveling surgery
Age 0 months-18 years old
Weight>2 kg
Cardiovascular criteria: Age-appropriate heart rate, mean arterial pressure and respiratory rate between the 10th and 90th percentile in the Pediatric Advanced Life Support (PALS) guidelines without any vasopressor, inodilator, inopressor or other pharmacologic support to achieve these criteria.
Pulmonary criteria: not intubated or reliant on pulmonary support such as a ventilator or administered oxygen unless diagnosed with CCHS who may be administered an FiO2 equivalent to room air
Presence of comorbid conditions that often accompany intestinal aganglionosis including Down's syndrome, Congenital Central Hypoventilation Syndrome or Waardenburg syndrome do not exclude candidates as long as above pulmonary and cardiovascular conditions are met.
Informed consent is given.

### Exclusion Criteria:

Short segment HD (At or below the rectosigmoid junction as assessed by biopsy)
Patients who do not meet the cardiovascular or pulmonary criteria above.
Structural heart defect except for patent foramen ovale or small ASD demonstrated on echocardiogram, to be obtained in all patients with Down's syndrome or with recorded heart murmurs.
Pregnancy confirmed by positive urine test and lactating mothers.
Renal failure with serum creatinine >3.0 or receiving chronic dialysis support.
History of significant recent bleeding disorder or coagulation profile (PT, INR, PTT) that are not within normal limits for the laboratory where measured.
Patients with known infectious disease (e.g., Hepatitis, HIV, TORCH, COVID-19, rotavirus, GBS, or other documented infection including positive blood or urine cultures, the appearance of pneumonia on chest X ray, or elevated WBC count above the laboratory limit where tested).
Patients with three times or more of the upper limits of normal hepatic enzymes above the laboratory limit where tested.
Patients with recent cerebrovascular accident or intracerebral hemorrhage of any grade.
Platelet count<50,000/ul or below the laboratory limit where tested
Neutropenia as defined by a level below the range for normal in the laboratory limits where tested
Prior history of malignancy
Any illness or diagnosis in which a reasonable clinician would have the opinion that immunosuppression is contraindicated.
Psychiatric illness, mental deficiency, or cognitive dysfunction which renders participation in treatment or informed consent impossible
Prior or ongoing exposure to permethrin or pyridostigmine bromide (agents known to lead to cell death of the ENS).
Any other medical condition, which, in the Investigator's judgment, will interfere with the patient's ability to comply with the protocol, compromises patient safety, or interferes with the interpretation of the study results

### Primary Endpoints

The primary endpoint of the clinical trial is safety, as measured by the frequency and severity of adverse events within 1 year (365 days) of ASCENT implantation that are related to the cells themselves, the procedure used to administer cells, and/or the concomitant immunosuppression administered.

To determine the occurrence of any of these adverse events, standard examinations will be applied to assess adverse events related to:
1) the injection of ASCENT including intestinal injury or bleeding, intestinal obstruction, or surgical site infection.
2) the ASCENT cells, including tumorigenicity.
3) the immunosuppression regimen, including systemic infection and evidence of sensitization.

### Secondary & Exploratory Endpoints

The secondary and exploratory endpoints of the trial will assess:
Motility changes of the treated intestine
High amplitude propagated contractions (HAPC/MMC), changes in Motility Index, and quantification of transit time.
Episodes of Hirschsprung's associated enterocolitis.
Internal anal sphincter pressure (IASP).
Recto-anal inhibitory reflex (RAIR).
The presence of ASCENT cells, neural cells, ganglia, or any aberrant tissue growth in biopsies of the ASCENT-treated intestine.

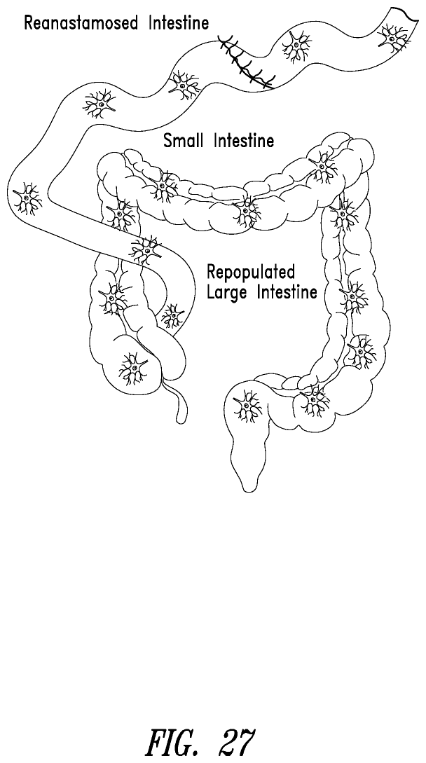

If injection of ASCENT is observed to increase motility to a physiologic level with the presence of ENS components on biopsy, in the future, connection of the intestine below the MF to the normal intestine can be considered in order to increase functional length (see FIG. 27).

### Study Design

The clinical trial will be a single arm, open label, dose escalation trial in pediatric patients diagnosed with LSA or TA who have received leveling surgery. Doses of 8-40×107 cells, treating up to 40 cm of intestine will be injected as described in the dosing section. Subjects will be followed 1 year for the primary safety endpoint and will be followed an additional year for long-term safety. Patients who do not consent to join the study will be asked to participate through chart review as standard of care controls, measuring any available data comparative to that obtained for the study population. There will be a staggered enrollment, with a 3-month observation period between the first and second subject to review procedures and understand immediate sequelae. DSMB oversight and approval will be required to progress to the second patient and at each subsequent subject. Upon DSMB review and approval, staggering after the second and subsequent subjects will be reduced to 1 month.

Subject number

6-10 subjects depending on recruitment of subjects with such a rare disease.

### Treatment Duration

ASCENT is intended to be permanent. Patients will receive cells in a one-time procedure under general anesthesia with immunosuppression started three days prior to injection and maintained after injection for as long as the ASCENT graft is supported. In the event of medical need, the treated intestine may be resected. If the treated intestine is resected or there is no evidence of functional components of the ENS, immunosuppression may cease.

### Duration of Follow Up

Subjects will be followed for one year for the primary safety endpoint and will be followed for an additional year for long-term safety. Clinical Examination: Patients will be examined daily until time of discharge with recorded vitals, abdominal exam, stool output and tacrolimus levels as well as for any side effects of immunosuppression. After discharge, patients will have office visits at 2 weeks, one month, 3 months, six months, nine months, and one year with a thorough physical examination and external assessment of the stoma and MF. Unscheduled visits will be conducted in the event of patient need. We will record time to intestinal transplant in this group as well as administer the Pediatric Quality of Life Inventory at one year

### Endoscopic Surveillance and Motility Testing:

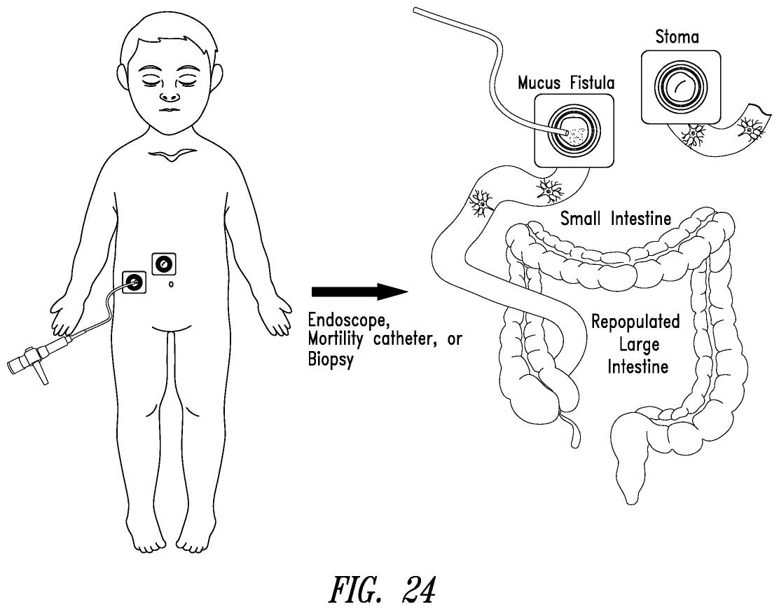

Both endoscopic monitoring with biopsy and motility testing will be performed through the MF at baseline, six months and one year after ASCENT (FIG. 24). Biopsies will be obtained at six months and one year and will be stained for ENS components. Specimens will be preserved in formalin and analyzed by a certified pathologist. Lab tests performed: Children with enteric neuropathies usually require total parenteral nutrition and are followed closely for nutritional and electrolyte balance, including twice weekly Chem 14 panels and frequent determination of blood albumin, transferrin, and hematocrit. Subjects in the trial will undergo the same lab testing. cPRA will be measured at one year to assess for sensitization, or earlier if intestinal transplant becomes necessary. If sensitization is identified, desensitization protocols could be considered prior to intestinal transplant, although this is not well guided by available data6.

### Dose Level(s) and Dose Justification

The final doses to be used in the clinical trial will be informed by the IND-enabling studies proposed in this application. However, it is currently anticipated that the starting dose of 8×107 cells will be distributed as 2 injections in the muscular coat of the intestine every 2 cm covering up to 8 cm of intestine (see FIG. 18). Subjects 2-5 will receive escalating doses of 16×107, 24×107, 32×107, and 40×107 cells, which will be distributed at the same injection density over 16, 24, 32 and 40 cm, respectively. Subjects 6-10 will receive the same dose as subject 5. All dosing will be performed by a pediatric surgeon using a 23-gauge needle.

### Justification of Clinical Dose

A dose of 1×107 cells in ImL will be administered to each side of every 2 cm of intestine up to a total of 40 cm in the muscular coat of the intestine.

We have chosen this dose because we have recorded good contractility in our HIO-TESI-delayed ASCENT model at both this dose and a dose one order of magnitude lower, 1×106 cells. Because the thickness of the host intestine may vary between patients and be thicker than that of HIO-TESI-delayed ASCENT, we elected for the higher effective dose that we tested. Bracketed doses will be tested in both rodent and swine models in the proposed studies and we will evaluate those data prior to a final dose determination.

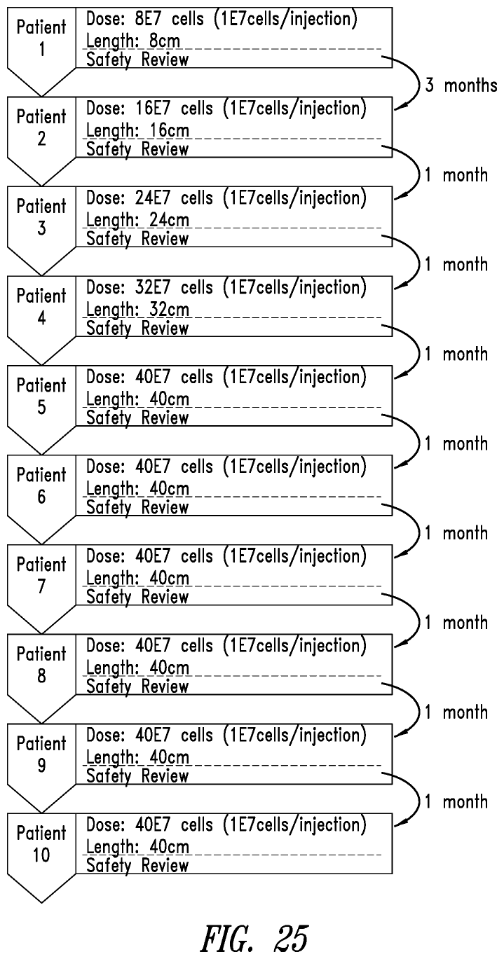

An illustrative staggered dose escalation is shown in FIG. 25.

### Route of Delivery

ASCENT will be thawed in the operating room with a controlled, qualified thawing method and drawn up with a large bore needle into a syringe. The needle will then be replaced by a 23G needle (Becton Dickinson 305145 or similar).

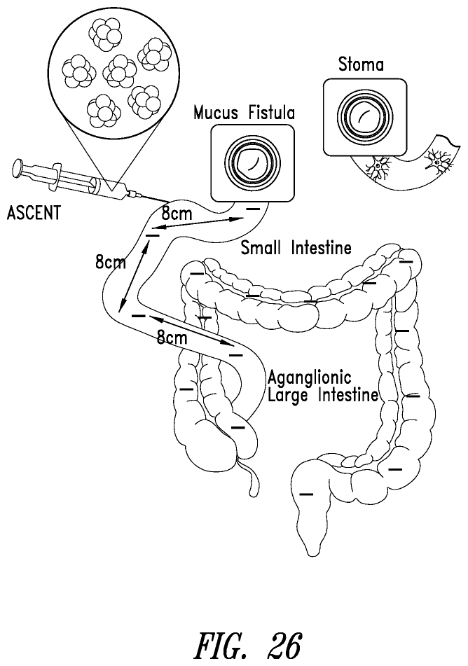

The injections will be administered as outlined in FIG. 26. The first subject will be administered a total of 8×107 cells over 8 cm (see above dosing scheme). Dose escalation for each subsequent subject will involve injection of an additional 8×107 cells along an additional 8 cm of the treated intestine (Figure Q) with the highest dose of 40×107 cells covering 40 cm. Standardized injection in the intestinal wall under direct visualization includes holding the needle in place for fifteen seconds after delivery to limit efflux. The injection procedure will deliver the cells in divided doses of ˜1×107 cells each in bilateral injections every 2 cm in the aganglionic intestine. The volume of each injection will be 1 mL. Injections will be graded as 0: cells not acceptably delivered, 1: some leakage at the injection site noted or 2: cells visualized as delivered in the appropriate location. Thaw to injection can be accomplished in under thirty minutes.

ASCENT is injected in a staggered dose/length protocol after confirming the level of enteric neuropathy.

If in follow-up, physiologic measurements of motility are accompanied by the presence of ENS components on biopsy, then reconnection to the patient's normally innervated intestine could be considered in conjunction with the DSMB, FDA, and parents of the subject. Even if the entire length of the aganglionic intestine is not fully populated, connection to an additional length of innervated intestine would be a better option than surgical resection particularly in the case of TA. FIG. 27 shows illustratively how the intestine can be eventually reconnected to natively ganglionated intestine (blue), after ASCENT has adequately repopulated the previously aganglionic segment with new components of the ENS (red).

### Data and Safety Monitoring Plan (DSMP)

An independent DSMB that does not include the study sponsor will be formed and review all study participants' progress and discuss any safety events. The DSMB will consist of 3-5 members and include a surgeon or gastroenterologist familiar with care of children with enteric neuropathy, an investigator with expertise in clinical trial methodology, and additional members who can 1) understand interim and cumulative data, 2) address concerns about safety and 3) adequately advise on the performance of the study, adherence to the protocol, and proposed protocol modifications (if any). The DSMB will review patient results and opine prior to proceeding to the next patient.

During this study the medical history, physical examinations, manometric investigations and all other procedures as noted above will be conducted according to current recommendations for subject privacy, and data will be kept in confidence following institutional guidelines. Adverse event reports and annual summaries will not include subject-identifiable information. Adverse events (AE) and serious adverse events (SAE) will be reported following the FDA definitions, and a classification plan will be included with an attribution scale. The consent form will include expected risks and measures taken to minimize risk. The DSMB will follow a charter for collecting, reporting, and follow-up of all SAEs. SAE reporting and follow-up will include timely reporting to the IRB, DSMB, FDA, and any other appropriate oversight bodies in the case of unexpected, serious, or intervention-related SAE. Protocol compliance will be reviewed on an ongoing basis with defined stopping rules.

### Stopping Rules

The stopping rules for this Phase I/2a clinical trial include development of an expanding mass in the treated intestine deemed of ASCENT origin, or development of any severe adverse pathology associated with the delivery, immunosuppression or use of ASCENT which warrants removal of the treated intestine.

### Immune Monitoring & Immunosuppression

Subjects will receive cells in a one-time procedure under general anesthesia with immunosuppression started three days prior to injection and maintained after injection for as long as the ASCENT graft is supported. If the distal intestine is resected or there is no evidence of functional components of the ENS, immunosuppression may cease. The immunosuppression regimen includes tacrolimus to achieve a target trough level of 15-20 ng/dL and thymoglobulin administered at a dose of 4 mg/kg. The tacrolimus will be dosed with target levels assessed weekly until steady state is accomplished, and then every two weeks until month 4, at which point levels can be determined monthly. Antihistamines will be administered prior to each dose of thymoglobulin. Before transplantation and for 24 hours to follow, patients will be administered ampicillin, gentamicin, and flagyl dosed/weight. In case of contraindication to thymoglobulin, either daclizumab (2mg/kg/wk) will be used for three months followed by 1 mg/kg/wk for another three months or alemtuzumab (0.3 mg/kg in four doses on days 0,1,3, and 7) post-transplant. Our maintenance tacrolimus levels will be trough concentrations of 15-20 ng/dL.

### Exploratory Studies

As noted above (FIG. 16), we will escalate our dose/length which will be particularly helpful to treat children with long sections of enteric neuropathy. We will look for evidence of differentiated components of the ENS on biopsy that migrate and establish networks over time following injection of ASCENT. We will also assess the changes from baseline in the compliance, propagated contractions and motility measurements. With adequate treated length, there may be improvements in anorectal manometry including RAIR.

### Assays Methodologies

Activity assessments include measuring changes in motility with clinically available standardized manometry that is readily available through the Motility Disorders Program at CHLA. This includes access to the computer software that allows analysis of the short-and long-term measurements, comparison to previous studies in the patient, and quantification. The Clinical Pathology department at CHLA regularly assesses patient specimens with ENS lesions and can analyze the endoscopic biopsies for all of the required ENS markers. Additional measurements of gene expression and cell engraftment from endoscopic biopsies will be undertaken in the Grikscheit lab under SOPs developed previously and reported in the Rationale Section.

### Sclatistical Analysis Plan

Although we would like to apply Bayesian guidelines for the clinical study design including stopping rules, the sample size will be small, and the patients varied, in order to ethically offer a new stem cell therapy that requires immunosuppression in pediatric patients. Therefore, our outcomes are more likely to be binary yes/no-motility measured where previously absent, ENS components newly identified in aganglionic intestine, functional and QALI measurements improved.

### Outcome Criteria

We seek to identify an acceptable safety profile with no obstruction, formation of teratomas, or SAEs related to the delivery of ASCENT. Restoration of even reduced intestinal motility in aganglionic intestine would be a marked improvement compared to current medical therapy and would avoid the severe morbidities of absent ENS function. Measurement of motility restoration by manometry, presence of ASCENT progeny by IF (any neurons or ganglia in intestinal segments that previously had none) and improvement on standard measures of manometry would all indicate success. We will report standardized instruments for these measurements.

### Potential Risks

1. Long-term immunosuppression has known risks that include cancers and infection because the immune system is less competent to detect both infectious agents and anomalous cells associated with various tumors. Tumors that are associated with viral infection such as non-Hodgkins lymphoma (Epstein-Barr virus) or liver cancer (hepatitis B virus) are increased in patients who receive long term immunosuppression. We have proposed a fairly aggressive immunosuppression protocol based on our experience with intestine transplants that convey a large amount of foreign immune tissue from the donor to the host, and we expect that ASCENT will be much less immunogenic. If we see successful survival of ASCENT after transplantation, it may be possible to reduce the target trough level of the tacrolimus in the future. We have chosen a fairly well tolerated and studied immunosuppressive agent, and patients with severe enteric neuropathies who go on to multiple surgeries to remove most of their native intestine prior to intestine transplant will be subjected to a much higher number of surgical procedures and the same or higher amounts of immunosuppression with poor success rates. Additionally, all of those procedures are more invasive and lengthier with higher surgical risks than intestine injections as we propose with ASCENT.

2. iPSC transplants can theoretically lead to the formation of teratomas from undifferentiated cells that are injected along with the differentiated cell product. In the case of teratomas after ASCENT engraftment, the treatment would be surgical excision of the initially aganglionic intestine into which ASCENT had been delivered. This resection is usually undertaken in these patients prior to intestinal transplant. We have never identified teratomas in any of our hundreds of small and large animals who have had ASCENT injected in various models. In the final product ASCENT, we do not detect the pluripotent stem cell markers Oct4 or Nanog.

Any surgical procedure carries risks of infection, allergic or other reaction to anesthesia, blood loss, or damage to surrounding organs or tissues.

### Clinical Sites

All injections in this study will be performed by Tracy Grikscheit, MD, Full Professor of Surgery and chief of the division of pediatric surgery at CHLA, who has also performed the majority of animal studies and therefore has familiarity with the product and delivery method. The study will be conducted in accordance with the International Committee for Harmonization-Good Clinical Practice guidelines and the Declaration of Helsinki. The cell delivery will be performed at Children's Hospital Los Angeles with IRB approval in the main OR at 4650 Sunset Boulevard, Los Angeles, CA, 90027, with informed consent. The trial will be preregistered.

### Enrollment

We will seek diverse representation in our clinical trials. Severe enteric neuropathies do not cohort in any particular ethnic group or gender, and usually result from spontaneous genetic mutations (although there are some familial cases of long segment HD) and are generally identified in neonates and referred to tertiary care pediatric surgery centers. We will recruit as outlined in our outreach activities with a staggered study in order to apply stopping rules.

### Long Term Follow Up

We will follow these patients in our colorectal clinic after the one-year follow-up as outlined above, CHLA sees patients regardless of ability to pay until age 21.

### Timeline

With nationwide (and in some cases international) referral of these patients, we expect to enroll 6-10 patients in eighteen months (see FIG. 25).

### Example 4

### Restoration of ENS Function In ASCENT-HIO-TESI

ASCENT neurons in vivo in the ASCENT-HIO-TESI model express excitatory (ChAT) and inhibitory (nNOS and GABA) neurotransmitters, as well as calbindin, which suggests development of sensory neurons: Myenteric neurons include excitatory and inhibitory motor neurons, descending and ascending interneurons, and intrinsic primary sensory neurons (46). We identified excitatory neurons (CHAT/TUJ1) and sensory neurons (5-HT/TUJ1) in ASCENT-HIO-TESI (41). We also observed calbindin/TUJ1 and calretinin/TUJ1 double positive neurons. Although a majority of calretinin-positive neurons are excitatory motor neurons, a small number represent intrinsic sensory neurons of the ENS (47, 48). Inhibitory neurons within the myenteric plexus and muscle fibers of the human small intestine (SI) and colon contain neuronal nitric oxide synthase (nNOS), which is the predominant isoform of NOS in the ENS and an integral component of the intestinal peristaltic reflex, secretion, digestion, absorption, and elimination. Production of nitric oxide results in relaxation of smooth muscle and controls the opening of sphincters, and mediates receptive and accommodative relaxation (49, 50, 51). It has been shown that the functional obstruction seen in HD patients may be caused by an inability to relax enteric smooth muscle (52). Co-implantation of ENCCs into developing HIO-TESI establishes inhibitory nNOS/TUJ1 double-positive neurons within the myenteric plexus². Taken together, these data identify the establishment of enteric ganglia within the submucosal and myenteric plexuses and engraftment of excitatory, inhibitory, and sensory neurons required for restoration of ENS function in ASCENT-HIO-TESI.

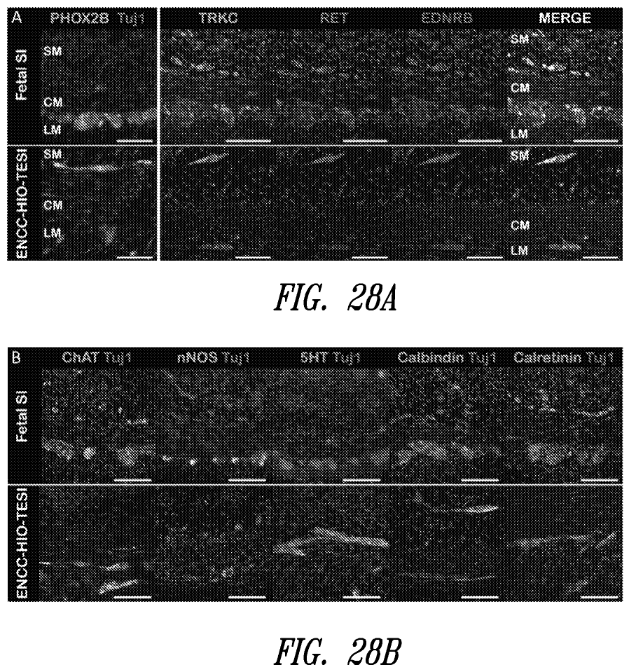

As shown in FIG. 28, vagal-specific ganglia and a wide array of neuronal subtypes are present in ASCENT-HIO-TESI immunofluorescent staining of human fetal ileum and ASCENT-HIO-TESI. A) TUJ1-positive submucosal and myenteric ganglia express vagal and enteric neural crest cell markers PHOX2B and TRKC/RET/EDNRB; submucosa (SM), circular muscle (CM), longitudinal muscle (LM). Scale bars, 100 mm (n=6). B) Various subclasses of enteric neurons were identified, including excitatory (CHAT/TUJ1), inhibitory (nNOS/TUJ1), and sensory neurons (5-HT/TUJ1). Calbindin- and calretinin-positive neurons were also restored in ASCENT-HIO-TESI: nuclei are labeled with DAPI (blue). Scale bars, 100 mm (n=11).

### Example 5

### Single Cell Expression Analysis

LiPSC-GR1.1 and Phox2^{-/-} cells were thawed and counted following the protocol listed below.

### Thaw Cells:

1. Place vial in 37C waterbath for 3 minutes until sliver of ice remains in vial.
2. Transfer thawed cells into 5 mL of Neurobasal media supplemented with N2.
3. Centrifuge cells for 3 minutes at 1000 rpm.
4. Aspirate supernatant.
5. Wash cells with 5 mL 1× PBS without Ca2⁺ or Mg2⁺.
6. Centrifuge cells for 3 minutes at 1000 rpm.
7. Aspirate supernatant.
8. Add 1 mL room temperature Accutase to cells. Place cells in 37C/5% CO₂ incubator for 3 minutes.
9. Check single cell dissociation by pipetting cells with 1 mL micropipette.
10. Deactivate Accutase with a minimum of 1.5× the Accutase volume of cold Neurobasal media supplemented with N2.
11. Thoroughly resuspend cells and transfer 20 uL to PCR tube for counting.

### Cell Counts:

1. Push the Power button to start the instrument. The Start-up screen is displayed.
2. Add 20 uL of 0.4% Trypan Blue and mix well.
3. Add 10 uL of Trypan stained cells to each side of the Countess Counting slide.
4. Insert the Countess™ cell counting chamber slide, sample side A first into the slide inlet on the instrument, making sure that the sample side A is inserted completely into the instrument. If the slide is pushed in correctly, there will be a soft click.
5. Press the Next Sample button.
6. Adjust the image by pressing the Zoom button. Navigate by pressing the location you like to see on the grid. Use the focus knob to adjust the image.
7. Press Count Cells.
8. Record the cell count in the “Count A” column.
9. Eject the slide by gently pressing the slide and releasing. Proceed to the B side of the slide. Count and record cells in “Count B” column.

### Calculating Cell Numbers

1. Average Total count A and Total Count B and record in “Average” column. Repeat for Live, Dead, and Viability counts. Total, Live, and Dead values are recorded as cells/mL.
2. Record Volume of counting sample (10 uL if no dilution factor).
3. Record volume of total cell suspension.
4. Multiply average count with total volume of cell suspension for total number of cells in cell suspension.

The following results were obtained:

| LIPSC- | | | | | Total in |
|---|---|---|---|---|---|
| GR1.1 | Count | Count | | Count | Cell |
| p41 | A | B | Average | volume | Suspension |
| Total | 2.90E+06 | 2.70E+06 | 2.80E+06 | 8 | 2.24E+07 |
| Live | 2.70E+06 | 2.50E+06 | 2.60E+06 | 8 | 2.08E+07 |
| Dead | 2.10E+05 | 2.30E+05 | 2.20E+05 | 8 | 1.76E+06 |
| Viability | 93% | 92% | 93% | | 93% |
| | | | | | Total in |
| Phox2B | Count | Count | | Count | Cell |
| p61 | A | B | Average | volume | Suspension |
| Total | 5.40E+06 | 5.80E+06 | 5.60E+06 | 8 | 4.48E+07 |
| Live | 4.60E+06 | 4.50E+06 | 4.55E+06 | 8 | 3.64E+07 |
| Dead | 7.80E+05 | 1.20E+05 | 4.50E+05 | 8 | 3.60E+06 |
| Viability | 85% | 78% | 82% | | 82% |

LiPSC-GR1.1 and Phox2B^{-/-} cells with >5e6 cells/vial were cryopreserved using the method below. Then, they were shipped to the Singulomics for single cell RNA sequencing.

### Cryopreservation

1. Prepare cryopreservation media: 1 mL of CryoStor CS10+10 uM ROCK inhibitor per vial. Keep at 4C until needed.
2. Label Cryovials with appropriate label.
3. Collect cells and centrifuge for 3 minutes at 1000 rpm at room temp.
4. Aspirate supernatant
5. Add cryopreservation medium to cell and thoroughly resuspend the cells.
6. Aliquot the cells in the appropriate volume for each vial.
7. Transfer vials to the 4C Mr. Frosty. Immediately transfer the Mr. Frosty to the −80C.
8. Allow to freeze overnight. Transfer within 48 hours to the LN2.

The raw data of scRNA sequencing which was generated was run through cell ranger software to identify individual gene features after aligning with a human genome database (GRCh38). Then the Loupe browser was selected to trim the data sets followed by the parameters listed below. These steps ensured data quality by removing dying cells and doublets.
UMIs: log2 2.5-16
Features: 2000-7000
Mitochondrial UMIs: <5%
PCA: 30

A total of 6 clusters were identified in the LiPSC-GR1.1 sample and their signature genes are listed below:

| Proliferating | | | | | |
|---|---|---|---|---|---|
| neuronal | Neural | | | | |
| intermediate | Progenitor | | Mesenchymal | | |
| progenitor cells | Cells | Neurons | stem cells | Glial cells | Fibroblasts |
| PCLAF | PIF1 | NHLH1 | BHLHE40 | APOD | SFRP1 |
| RRM2 | PLK1 | NHLH2 | SLC16A3 | PRSS56 | MCM6 |
| TK1 | NEK2 | ELAVL4 | GDF15 | HEY2 | ID4 |
| FEN1 | AURKA | INSM1 | WFDC2 | ADGRG6 | PRSS23 |
| CLSPN | CENPA | NEUROD1 | HSPA1A | MPZ | FOSB |
| RAD51AP1 | UBE2C | VXN | DDIT4 | MGLL | |
| CHAF1A | CCNB1 | KLHL35 | SLC2A3 | S100B | |
| ASF1B | CDCA3 | NEFM | P4HA1 | FOXD3-AS1 | |
| DHFR | HMMR | SYT4 | LDHA | MIA | |
| DUT | PSRC1 | STMN2 | LGALS1 | A2M | |
| PCNA | SGO2 | NEUROG1 | CDKN1A | BGN | |
| ZWINT | TTK | ONECUT2 | HSPA1B | LINC01198 | |
| MCM4 | FAM83D | TMEM176A | DLK1 | SOX10 | |
| TYMS | ASPM | DLL3 | MDM2 | ERBB3 | |
| CENPU | GTSE1 | TMEM176B | BNIP3 | SPP1 | |
| CENPK | DLGAP5 | SRRM4 | PNCK | BCAN | |
| ORC6 | CDC20 | SST | WSB1 | LUM | |
| UHRF1 | CENPE | PCSK1N | FAM 162A | ATP1A2 | |
| UNG | KNSTRN | SSTR2 | RAB3A | ECEL1 | |
| HELLS | CKAP2L | ELAVL3 | DNAJB1 | ALDH1A1 | |
| RFC2 | TOP2A | GADD45G | PGK1 | RGCC | |
| RFC4 | CDCA8 | RASD1 | ENO2 | PLAT | |
| RRM1 | CDKN3 | RGS16 | STC2 | PLP1 | |
| GINS2 | KIF23 | PHLDA1 | ALDOA | COL1A2 | |
| GMNN | TPX2 | PPP1R14C | GPI | TFAP2B | |
| CDCA4 | NUF2 | NEFL | H1FO | SH3KBP1 | |
| DNAJC9 | TROAP | TAGLN3 | IGFBP2 | CDH6 | |
| PXMP2 | NDC80 | CRABP1 | C1orf54 | SCRG1 | |
| SLBP | CCNA2 | ROBO3 | FKBP4 | FGFBP3 | |
| FBXO5 | PRC1 | FAM57B | SCARB2 | RCAN1 | |
| MCM3 | CENPF | CNTN2 | HSPB1 | CD74 | |
| RPA2 | PTTG1 | DCX | ZMAT3 | COL9A1 | |
| CHEK1 | PIMREG | FAM110A | DUSP15 | NRP2 | |
| HSPB11 | KIF2C | STMN4 | NGFR | PLEKHA4 | |
| CDT1 | AURKB | ARL4D | NEAT1 | ARPC1B | |
| VRK1 | TACC3 | CDKN1C | ETFB | PDLIM4 | |
| MCM7 | RACGAP1 | TFAP2B | ATP6V1H | NID1 | |
| CASP8AP2 | ECT2 | THSD7A | SPINT2 | SMOC1 | |
| CENPM | MKI67 | INA | SLC2A1 | EGR2 | |
| HIST1H4C | ARL6IP1 | RAB3A | FAM122B | C9orf3 | |
| HAT1 | KPNA2 | GLRX | RRAGD | CDC42EP5 | |
| MCM5 | CDK1 | DLL1 | FDXR | FBLN1 | |
| USP1 | UBE2S | GAP43 | ACTA2 | PHLDA1 | |
| SMC2 | KIFC1 | ELAVL2 | SLC44A2 | TIMP3 | |
| ACYP1 | PBK | HES6 | H3F3B | RGS10 | |
| PPIF | SGO1 | JAG1 | LGMN | PTN | |
| ARL6IP6 | KIF11 | FNDC5 | BNIP3L | STRA6 | |
| MTHFD2 | NUSAP1 | PCBP4 | POLR2H | XPNPEP1 | |
| SVIP | CKAP2 | RGMB | CCNG2 | IGFBP5 | |
| TFDP1 | NCAPG | MLLT11 | NAMPT | RARRES2 | |
| SMC1A | KIF20B | MIAT | WDR45B | GSTO1 | |
| WDR34 | DEPDC1B | GPC2 | SNCG | HES1 | |
| EXOSC9 | TUBA1C | MAP2 | PRDX6 | ANXA2 | |
| MCM6 | CCNB2 | NGFR | MIF | SPTBN1 | |
| SAC3D1 | TUBB4B | NSG1 | TMEM256 | ADD3 | |
| DNMT1 | BIRC5 | RTN1 | EIF4H | KLF6 | |
| ITGB3BP | SPC25 | KIF5C | CHCHD5 | LITAF | |
| RPA3 | H2AFX | SCG3 | UNC119 | LEF1 | |
| MND1 | RTKN2 | DST | | PIK3R1 | |
| UBE2T | G2E3 | RND3 | | CXCR4 | |
| SPC25 | MIS18BP1 | SYT1 | | NET1 | |
| POLR3K | CKS1B | MAP1B | | DNAJC1 | |
| RANBP1 | CKS2 | TUBB3 | | RHOB | |
| MCM2 | KIF22 | MAP1A | | NR4A1 | |
| CTNNAL1 | OIP5 | NOVA1 | | NKAIN4 | |
| MSH2 | MAD2L1 | NKAIN4 | | LMO4 | |
| EIF4EBP1 | HMGB2 | RUFY3 | | GYPC | |
| SERPINE2 | CKAP5 | SNCG | | CNP | |
| COMMD4 | SMC4 | CHRNA5 | | OPN3 | |
| CENPX | BUB3 | NCAM1 | | PPP1R18 | |
| TMEM106C | MZT1 | ENC1 | | CELF2 | |
| GGCT | CENPW | RBFOX2 | | SESN3 | |
| SIVA1 | DBF4 | IGFBP5 | | MPG | |
| CENPH | FAM110A | BASP1 | | SERPINE2 | |
| C19orf48 | DDX39A | SEZ6L2 | | CUEDC2 | |
| HNRNPF | RHNO1 | SOX11 | | GLUL | |
| EZH2 | UBE2T | DAAM1 | | PCBP4 | |
| MAD2L1 | NUDCD2 | PBX3 | | MID1IP1 | |
| RARRES2 | FAM122B | RBP1 | | HEXB | |
| NETO2 | DTYMK | WFIKKN1 | | CREB5 | |
| POLE3 | RAD21 | TUBB2A | | WFDC2 | |
| AIF1L | CENPN | SH3BGRL3 | | GLTP | |
| PGP | AC084033.3 | TERF2IP | | FBXO7 | |
| CSE1L | NMU | GDI1 | | TMSB10 | |
| IDH2 | CCDC34 | SESN3 | | TSN | |
| SRSF2 | LBR | GDAP1L1 | | LGALS1 | |
| DEK | NUCKS1 | CPE | | ARL6IP5 | |
| | CENPM | FGD4 | | NIFK | |
| | HIST1H4C | TUBA1A | | NR2F2 | |
| | MND1 | PKIA | | SVIP | |
| | HMGB3 | OCIAD2 | | FTL | |
| | FBXO5 | ATP1B1 | | LAPTM4A | |
| | DCAF7 | PGM2L1 | | SELENOW | |
| | SMC2 | CADM1 | | SQSTM1 | |
| | NUP37 | MIR124-2HG | | LY6E | |
| | H1FX | SRGAP3 | | ZFP36L2 | |
| | MIS18A | HOXB8 | | CD164 | |
| | VRK1 | SYT11 | | MINDY2 | |
| | FRZB | SOX4 | | TAX1BP3 | |
| | HSP90B1 | LRRN1 | | COL2A1 | |
| | SAP30 | LAMP5 | | RAB34 | |
| | ADD3 | FBLN1 | | ENC1 | |
| | TMPO | EPB41 | | NCALD | |
| | TGIF1 | CCND2 | | EFNB2 | |
| | ANP32E | PROX1 | | VGLL4 | |
| | TUBA1B | ZBTB20 | | AIF1L | |
| | HP1BP3 | CAMK2N1 | | UBXN2A | |
| | CALM2 | TMSB10 | | CTSB | |
| | NUDT1 | KLC1 | | TLE1 | |
| | SKA2 | TCF12 | | NUDT4 | |
| | TMEM60 | TMSB4X | | PPP1R15A | |
| | RUVBL2 | ENO2 | | TRIM8 | |
| | LMNB1 | GRAMD1A | | | |
| | H2AFZ | LINC01933 | | | |
| | DCTN3 | PPP1R18 | | | |
| | HMGB1 | MIB1 | | | |
| | JPT1 | FJX1 | | | |
| | RNF5 | RTN4 | | | |
| | H2AFV | MAP6 | | | |
| | MYEF2 | CRMP1 | | | |
| | BCL2L12 | HOXB5 | | | |
| | CDCA4 | GDF11 | | | |
| | ZWINT | UBE2L6 | | | |
| | RPL39L | GADD45A | | | |
| | PNRC2 | COTL1 | | | |
| | ASF1B | GRB2 | | | |
| | ARL4C | BCL7A | | | |
| | DYNLL1 | BEX2 | | | |
| | HMGN2 | YPEL5 | | | |
| | CCDC167 | TUBB2B | | | |
| | HDGF | FYN | | | |
| | TXNDC12 | TCF4 | | | |
| | SSNA1 | MAPK6 | | | |
| | TK1 | DPYSL4 | | | |
| | PPP2R5C | AP1S2 | | | |
| | FZD2 | FNBP1L | | | |
| | AKIRIN2 | MAP4K4 | | | |
| | RUVBL1 | KLHDC8A | | | |
| | HMGA2 | RNF24 | | | |
| | HNRNPA2B1 | RGS2 | | | |
| | IRX2 | RCOR2 | | | |
| | EXOSC8 | TBPL1 | | | |
| | MORF4L2 | CD24 | | | |
| | ID1 | CCNG2 | | | |
| | PSPC1 | PROX1 | | | |
| | MRPL51 | ZBTB20 | | | |
| | | CAMK2N1 | | | |
| | | TMSB10 | | | |
| | | KLC1 | | | |
| | | TCF12 | | | |
| | | TMSB4X | | | |
| | | ENO2 | | | |
| | | GRAMD1A | | | |
| | | LINC01933 | | | |
| | | PPP1R18 | | | |
| | | MIB1 | | | |
| | | FJX1 | | | |
| | | RTN4 | | | |
| | | MAP6 | | | |
| | | CRMP1 | | | |
| | | HOXB5 | | | |
| | | GDF11 | | | |
| | | UBE2L6 | | | |
| | | GADD45A | | | |
| | | COTL1 | | | |
| | | GRB2 | | | |
| | | BCL7A | | | |
| | | BEX2 | | | |
| | | YPEL5 | | | |
| | | TUBB2B | | | |
| | | FYN | | | |
| | | TCF4 | | | |
| | | MAPK6 | | | |
| | | DPYSL4 | | | |
| | | AP1S2 | | | |
| | | FNBP1L | | | |
| | | MAP4K4 | | | |
| | | KLHDC8A | | | |
| | | RNF24 | | | |
| | | RGS2 | | | |
| | | RCOR2 | | | |
| | | TBPL1 | | | |
| | | CD24 | | | |
| | | CCNG2 | | | |
| | | ATP6V1E1 | | | |
| | | RHOBTB3 | | | |
| | | DACH1 | | | |
| | | TCEAL7 | | | |
| | | RNF165 | | | |
| | | STMN1 | | | |
| | | MEIS2 | | | |
| | | KCNQ1OT1 | | | |
| | | PLXNA2 | | | |
| | | YWHAG | | | |
| | | RNF11 | | | |
| | | CALM2 | | | |
| | | MAP1LC3B | | | |
| | | SEPT6 | | | |
| | | CHMP1B | | | |
| | | RAB6A | | | |
| | | NREP | | | |
| | | STARD3NL | | | |
| | | SSBP3 | | | |
| | | ATP6V1H | | | |
| | | EVL | | | |
| | | C3orf14 | | | |
| | | CSNK1E | | | |
| | | BAZ2B | | | |
| | | ATAT1 | | | |
| | | TRAF4 | | | |
| | | ARMCX3 | | | |
| | | TLE1 | | | |
| | | TIMM50 | | | |
| | | HOXB4 | | | |
| | | RPAIN | | | |
| | | TSG101 | | | |
| | | CYTH2 | | | |
| | | NDUFB8 | | | |
| | | DPYSL3 | | | |
| | | TSPAN13 | | | |
| | | ASCL1 | | | |
| | | SEC11C | | | |
| | | HOXB3 | | | |
| | | CLTB | | | |
| | | RERE | | | |
| | | CXCR4 | | | |
| | | HOXB2 | | | |
| | | CLDND1 | | | |
| | | LIMD2 | | | |
| | | ZEB1 | | | |
| | | KDM5B | | | |
| | | BNIP3 | | | |
| | | SPAG9 | | | |
| | | ATP6V0B | | | |
| | | DYNC1H1 | | | |
| | | DBN1 | | | |

Based on these results, it can be seen that a combination of cell types and gene signatures including pluripotent, neuronal and ganglia markers as well as less differentiated/earlier cell types are present in ENCCs produced in some of the methods of the disclosure. Further, in some cases, such a combination of cell types is believed to be important in leading to the successful formation of ENS structures in vivo following administration of the cells.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, embodiments, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

### REFERENCES

1. Baghbaderani B A, Tian X, Neo B H, et al. cGMP-Manufactured Human Induced Pluripotent Stem Cells Are Available for Pre-clinical and Clinical Applications. Stem Cell Reports 2015:5:647-59.

2. Baghbaderani B A, Syama A, Sivapatham R, et al. Detailed Characterization of Human Induced Pluripotent Stem Cells Manufactured for Therapeutic Applications. Stem Cell Rev 2016:12:394-420.

3. Amiel J S-EE, Garcia-Barcelo M, et al. Hirschsprung disease, associated syndromes and genetics: a review. Journal of Medical Genetics 2008:11-14.

4. Chen S H. In: Stefan Scholz M D, ed. Volume 2017.

5 Parisi M A. Hirschsprung Disease Overview. In: Pagon R A, Adam M P, Ardinger H H, Wallace S E, Amemiya A, Bean L J H, Bird T D, Ledbetter N, Mefford H C, Smith R J H, Stephens K, eds. GeneReviews(R). Seattle (WA), 1993.

6. Lee G, Kim H, Elkabetz Y, et al. Isolation and directed differentiation of neural crest stem cells derived from human embryonic stem cells. Nat Biotechnol 2007:25:1468-75.

7 Cline G W, Johnson K, Regittnig W, et al. Effects of a novel glycogen synthase kinase-3 inhibitor on insulin-stimulated glucose metabolism in Zucker diabetic fatty (fa/fa) rats. Diabetes 2002:51:2903-10.

8. Fattahi F, Steinbeck J A, Kriks S, et al. Deriving human ENS lineages for cell therapy and drug discovery in Hirschsprung disease. Nature 2016:531:105-9.

9. Mica Y, Lee G, Chambers S M, et al. Modeling neural crest induction, melanocyte specification, and disease-related pigmentation defects in hESCs and patient-specific iPSCs. Cell Rep 2013:3:1140-52.

10. Sasselli V, Pachnis V, Burns A J. The enteric nervous system. Dev Biol 2012:366:64-73.

11. Mckeown S J, Mohsenipour M, Bergner A J, et al. Exposure to GDNF Enhances the Ability of Enteric Neural Progenitors to Generate an Enteric Nervous System. Stem Cell Reports 2017:8:476-488.

12. Mwizerwa O, Das P, Nagy N, et al. Gdnf is mitogenic, neurotrophic, and chemoattractive to enteric neural crest cells in the embryonic colon. Dev Dyn 2011:240:1402-11.

13. Lee S H, Lumelsky N, Studer L, et al. Efficient generation of midbrain and hindbrain neurons from mouse embryonic stem cells. Nat Biotechnol 2000:18:675-9.

14. Schlieve C R, Fowler K L, Thornton M, et al. Neural Crest Cell Implantation Restores Enteric Nervous System Function and Alters the Gastrointestinal Transcriptome in Human Tissue-Engineered Small Intestine. Stem Cell Reports 2017:9:883-896.

15. Miyaoka Y, Chan A H, Judge L M, et al. Isolation of single-base genome-edited human iPS cells without antibiotic selection. Nat Methods 2014:11:291-3.

16. Zimmer J, Puri P. Knockout mouse models of Hirschsprung's disease. Pediatr Surg Int 2015:31:787-94.

17. Laboratory T J. Mouse Strain Datasheet-021933, B6.129S7-Ednrbtm1Ywa/FrykJ. Volume 2017.

18. Frykman P K, Cheng Z, Wang X, et al. Enterocolitis causes profound lymphoid depletion in endothelin receptor B- and endothelin 3-null mouse models of Hirschsprung-associated enterocolitis. Eur J Immunol 2015:45:807-17.

19. Wieck M M, El-Nachef W N, Hou X, et al. Human and Murine Tissue-Engineered Colon Exhibit Diverse Neuronal Subtypes and Can Be Populated by Enteric Nervous System Progenitor Cells When Donor Colon Is Aganglionic. Tissue Eng Part A 2016:22:53-64.

20. Zhao L, Cheng Z, Dhall D, et al. A novel corrective pullthrough surgery in a mouse model of Hirschsprung's disease. J Pediatr Surg 2009:44:759-66.

21. Spence J R, Mayhew C N, Rankin S A, et al. Directed differentiation of human pluripotent stem cells into intestinal tissue in vitro. Nature 2011:470:105-9.

22. Thomson J A, Itskovitz-Eldor J, Shapiro S S, et al. Embryonic stem cell lines derived from human blastocysts. Science 1998:282: 1145-7.

23. Workman M J, Mahe M M, Trisno S, et al. Engineered human pluripotent-stem-cell-derived intestinal tissues with a functional enteric nervous system. Nat Med 2017:23:49-59.

24. Finkbeiner S R, Freeman J J, Wieck M M, et al. Generation of tissue-engineered small intestine using embryonic stem cell-derived human intestinal organoids. Biol Open 2015:4:1462-72.

25. Sala F G, Matthews J A, Speer A L, et al. A multicellular approach forms a significant amount of tissue-engineered small intestine in the mouse. Tissue Eng Part A 2011:17:1841-50.

26. McCracken K W, Howell J C, Wells J M, et al. Generating human intestinal tissue from pluripotent stem cells in vitro. Nat Protoc 2011:6:1920-8.

27. Furness J B. The enteric nervous system and neurogastroenterology. Nat Rev Gastroenterol Hepatol 2012:9:286-94.

28. Furness J B, Callaghan B P, Rivera L R, et al. The enteric nervous system and gastrointestinal innervation: integrated local and central control. Adv Exp Med Biol 2014:817:39-71.

29. Uesaka T, Young H M, Pachnis V, et al. Development of the intrinsic and extrinsic innervation of the gut. Dev Biol 2016.

30. Furness J B, Johnson P J, Pompolo S, et al. Evidence that enteric motility reflexes can be initiated through entirely intrinsic mechanisms in the guinea-pig small intestine. Neurogastroenterol Motil 1995:7:89-96.

31. Liu L W, Thuneberg L, Huizinga J D. Selective lesioning of interstitial cells of Cajal by methylene blue and light leads to loss of slow waves. Am J Physiol 1994:266:G485-96.

32. Dean S K, Horley D C, Tuch B E. The immunosuppression of pigs: a review. Transplantation Reviews 2003:17:45-59.

33. Allison A C, Eugui E M. Mycophenolate mofetil and its mechanisms of action. Immunopharmacology 2000:47:85-118.

34. Boddington S, Henning T D, Sutton E J, et al. Labeling stem cells with fluorescent dyes for non-invasive detection with optical imaging. J Vis Exp 2008.

35. Lee G, et al. Modelling pathogenesis and treatment of familial dysautonomia using patient-specific iPSCs. Nature. 2009: 461:402-406.

36. Zeltner N, Lafaille F G, Fattahi F, Studer L. Feeder-free derivation of neural crest progenitor cells from human pluripotent stem cells. Journal of visualized experiments.: JoVE. 2014.

37. Lake and Heuckeroth. (2013). Enteric nerous system development: migration, differentiation, and disease. Am. J. Physiol. Gastrointest. Liver Physiol. 305, G1-G24.

38. Laughlin et al. (2012). Total colonic aganglionosis: a systematic review and meta-analysis of long-term clinical outcome. Pediatr. Surg. Int. 28, 773-779.

39. Sulkowski et al. (2014). Single-stage versus multi-stage pull-through for Hirschsprung's disease: practice trends and outcomes in infants. J. Pediatr. Surg. 49, 1619-1625.

40. Lee, H. T. et al. The mechanism and spread of pacemaker activity through myenteric interstitial cells of Cajal in human small intestine. Gastroenterology 132, 1852-1865 (2007).

41. Schlieve, C. R. et al. Neural Crest Cell Implantation Restores Enteric Nervous System Function and Alters the Gastrointestinal Transcriptome in Human Tissue-Engineered Small Intestine. Stem Cell Reports 9, 883-896 (2017).

42. Furness, J. B., Rivera, L. R., Cho, H. J., Bravo, D. M. & Callaghan, B. The gut as a sensory organ. Nat Rev Gastroenterol Hepatol 10, 729-740 (2013).

43. Bohorquez, D. V. et al. Neuroepithelial circuit formed by innervation of sensory enteroendocrine cells. The Journal of clinical investigation 125, 782-786 (2015).

44. Boddington, S. E. et al. Labeling human embryonic stem cell-derived cardiomyocytes with indocyanine green for noninvasive tracking with optical imaging: an FDA-compatible alternative to firefly luciferase. Cell transplantation 19, 55-65 (2010). 45. Santeusanio, A. D., Moon, J., Nair, V. & Iyer, K. R. Is There a Role for Desensitization in Intestinal Transplantation? Prog Transplant 29, 275-278 (2019).

46. Costa, M., Brookes, S. J. & Hennig, G. W. Anatomy and physiology of the enteric nervous system. Gut 47 Suppl 4, iv15-19; discussion iv26 (2000).

47. Kunze, W. A. & Furness, J. B. The enteric nervous system and regulation of intestinal motility. Annu Rev Physiol 61, 117-142 (1999).

48. Qu, Z. D. et al. Immunohistochemical analysis of neuron types in the mouse small intestine. Cell Tissue Res 334, 147-161 (2008).

49. Beck, M., Schlabrakowski, A., Schrodl, F., Neuhuber, W. & Brehmer, A. ChAT and NOS in human myenteric neurons: co-existence and co-absence. Cell Tissue Res 338, 37-51 (2009).

50. Takahashi, T. Pathophysiological significance of neuronal nitric oxide synthase in the gastrointestinal tract. J Gastroenterol 38, 421-430 (2003).

51. Timmermans, J. P. et al. Nitric oxide synthase immunoreactivity in the enteric nervous system of the developing human digestive tract. Cell Tissue Res 275, 235-245 (1994).

52. Furness, J. B. The enteric nervous system and neurogastroenterology. Nat Rev Gastroenterol Hepatol 9, 286-294 (2012).

1. A method to treat an enteric neuropathy comprising administering enteric neural crest cells (ENCCs) to a muscular wall of the intestine in a subject in need thereof, wherein the ENCCs are differentiated from stem cells in vitro.

2. The method of claim 1, wherein the enteric neuropathy is Hirschsprung's Disease (HD)

3. The method of claim 1, wherein the enteric neuropathy is a total intestinal aganglionosis form of HD or a long segment forms of HD.

4. The method of claim 1, wherein the enteric neuropathy is aganglionosis of the rectosigmoid colon (short segment disease), aganglionosis of the sigmoid colon (long segment disease), congenital aganglionosis in Hirschsprung disease (HD), autoimmune-mediated loss of neuronal subtypes in esophageal achalasia and Chagas disease, degenerative neuropathies in chronic intestinal pseudo-obstruction or gastroparesis, enteric neuropathy caused by nerve agents (e.g., Gulf War syndrome) and/or diabetic gastroparesis.

5. The method of claim 1, wherein the entire intestine is aganglionic.

6. The method of claim 1, wherein the cells are administered by injection.

7. The method of claim 1, wherein the cells are administered to two or more different locations of the intestine.

8. The method of claim 1, wherein the cells are administered by bilateral injections about every 1 to 3 centimeters in the muscular wall of the intestine.

9. The method of claim 8, wherein the cells are administered about every 2 centimeters in the muscular wall of the intestine.

10. The method of claim 8, wherein two or more doses of cells are administered about every 2 cm in the muscular wall of the intestine.

11. The method of claim 8, wherein each dose comprises about 1×10⁵ to 1×10⁹ cells.

12. The method of claim 11, wherein each dose comprises about 1×10⁷ cells.

13. The method of claim 1, wherein the cells are suspended in a pharmaceutically acceptable carrier.

14. The method of claim 1, wherein the cells are cryopreserved and thawed prior to administration to said subject.

15. The method of claim 1, further comprising administering one or more additional agents selected from the group consisting of a preservative, a cytokine, a pH buffering agent and a migration promoting agent, or a combination thereof.

16. The method of claim 15, wherein the migration promoting agent is selected from the group consisting of Glial cell-derived neurotrophic factor (GDNF), Brain-derived neurotrophic factor (BDNF) and Nerve growth factor (NGF), or a combination thereof.

17. The method of claim 1, wherein the subject has been clinically immunosuppressed prior to administration of the cells.

18. The method of claim 1, wherein the stem cells are induced pluripotent stem cells or embryonic stem cells.

19. The method of claim 18, wherein the induced pluripotent stem cells comprise the cell line LiPSC-GR1.1.

20. The method of claim 1, wherein the cells are allogeneic to said subject.

21. The method of claim 1, wherein the cells are genetically modified.

22. The method of claim 21, wherein the cells are genetically modified to evade immune system surveillance.

23. A composition comprising enteric neural crest cells (ENCCs) differentiated from stem cells in vitro and a cryopreservative.

24. The composition of claim 23, wherein the stem cells comprise induced pluripotent stem cells or embryonic stem cells.

25. The composition of claim 24, wherein the induced pluripotent stem cells comprise the cell line LiPSC-GR1.1.

26. A neurosphere comprising a heterogeneous mixture of cells, wherein the mixture of cells are positive for expression HNK-1, positive for expression p75, negative for expression Oct4 and negative for expression Nanog, wherein the expression of HNK-1, p75, Oct4 and/or Nanog is from one cell or each marker is expressed individually or in combination on different cells.

27. A method for producing enteric neural crest cells (ENCCs) comprising the steps of:
a) expanding stem cells in suspension;
b) contacting the stem cells of a) with at least one inhibitor of SMAD, at least one activator of the WNT pathway, FGF2 and retinoic acid and culturing said stem cells for a period of time and under conditions sufficient to differentiate said stem cells to enteric neural crest cells (ENCCs); and
c) isolating said enteric neural crest cells (ENCCs) of b).

28. The method of claim 27, wherein the stem cells are iPSCs.

29. The method of claim 28, wherein the iPSCs comprise the cell line LiPSC-GR1.1.

30. The method of claim 27, wherein the inhibitor of SMAD is LDN193189 and/or SB431542, or an analog thereof.

31. The method of claim 27, wherein the activator of the WNT pathway is CHIR99021 or an analog thereof.

32. The method of claim 27, wherein the stem cells are a) cultured in medium with at least one inhibitor of SMAD and FGF2 for a period of time,
b) the cells of a) are then cultured in a medium with at least one inhibitor of SMAD, FGF2 and at least one activator of the WNT pathway for a period of time;
c) the cells of b) are cultured then cultured in a medium with at least one inhibitor of SMAD, FGF2, at least one activator of the WNT pathway and retinoic acid for a period of time;
d) the cells of c) are then cultured in a medium with at least one inhibitor of SMAD, at least one activator of the WNT pathway and retinoic acid for a period of time; and e) the cells of d) are then cultured in a medium with FGF2 and at least one activator of the WNT pathway for a period of time.

33. The method of claim 27, wherein the isolated enteric neural crest cells (ENCCs) are cryopreserved prior to administration.

34. Enteric neural crest cells (ENCCs) made according to the method of claim 27.

35. The enteric neural crest cells (ENCCs) of claim 34, wherein the cells comprise a substantially homogeneous population of cells.

36. The enteric neural crest cells (ENCCs) of claim 34, wherein the cells comprise a heterogeneous population of cells.

37. A pharmaceutical composition comprising enteric neural crest cells (ENCCs) of claim 34 and a pharmaceutically acceptable carrier.

38. A method for treatment an enteric neuropathy comprising injecting the enteric neural crest cells (ENCCs) of claim 34 to a muscular wall of the intestine of a subject in need thereof.
